(19) United States
(12) Patent Application Publication
(10) Pub. No.: US 2011/0189232 A1
(43) Pub. Date: Aug. 4, 2011

(54) RECOMBINANT HUMAN PARAINFLUENZA TYPE 1 VIRUSES (HPIV1s) CONTAINING MUTATIONS IN OR DELETION OF THE C PROTEIN ARE ATTENUATED IN AFRICAN GREEN MONKEYS AND IN CILIATED HUMAN AIRWAY EPITHELIAL CELLS AND ARE POTENTIAL VACCINE CANDIDATES FOR HPIV1

(75) Inventors: Emmalene Bartlett, Chevy Chase, MD (US); Peter L. Collins, Silver Spring, MD (US); Mario H. Skiadopoulos, Potomac, MD (US); Brian R. Murphy, Bethesda, MD (US)

(73) Assignee: The Government of the United States of America Represented by the Secretary, Rockville, MD (US)

(21) Appl. No.: 13/001,710

(22) PCT Filed: Jul. 1, 2009
(86) PCT No.: PCT/US09/49461

§ 371 Date: Apr. 18, 2011

Related U.S. Application Data
(62) Division of application No. 61/077,483, filed on Jul. 1, 2008.

(51) Int. Cl. A61K 39/155; C12N 7/04; C12N 15/63; C07H 21/00; C12N 5/10; A61P 37/04; A61P 31/14

(52) U.S. Cl. 424/211.1; 435/236; 435/320.1; 536/23.72; 435/325

(57) ABSTRACT

Two recently characterized live attenuated HPIV1 vaccine candidates, rHPIV1-C^{R84G/Δ170}HN^{T553A}L^{Y942A} and rHPIV1-C^{R84G/Δ170}HN^{-T553A}L^{Δ1710-11}, which contain temperature sensitive (ts) attenuating (att) and non-ts att mutations, were evaluated in a Human Airway Epithelium (HAE) model culture system and in vivo in African Green monkeys (AGM). The vaccine candidates were highly restricted in growth in HAE at permissive (32° C.) and restrictive (37° C.) temperatures. The viruses grew slightly better at 37° C. than at 32° C., and rHPIV1-C^{R84G/Δ170}HN^{T553A}-L^{Y942A} was less attenuated than rHPIV1-CR^{84G/Δ170}HN^{T553A}L^{Δ1710-11}. The level of replication in HAE correlated with that observed in African Green monkeys, suggesting that the HAE model is useful as a tool for pre-clinical evaluation of HPIV1 vaccines. A live attenuated HPIV1 vaccine candidate having a normal P/C gene structure of overlapping P and C open reading frames, but does not express any functional C protein, is found to highly attenuated in AGMs, and provides a significant immune response in AGMs.

### STATEMENT OF GOVERNMENT INTEREST

The invention described herein was made in part with funding by the government of the United States of America. The United States government may retain certain rights in the subject invention of this application.

### FIELD OF THE INVENTION

The present invention relates to infectious, recombinant, self-replicating attenuated human parainfluenza virus type 1 (HPIV1) particles and their corresponding encoding polynucleotides able. The present invention further relates to using the infectious, recombinant, self-replicating attenuated human parainfluenza virus type 1 (HPIV1) particles and their corresponding encoding polynucleotides to make vaccines for use in mammalian subjects, including humans.

### BACKGROUND OF THE INVENTION

Human parainfluenza viruses are enveloped, non-segmented, single-stranded, negative-sense RNA viruses belonging to the family Paramyxoviridae. This group of viruses includes HPIV serotypes 1, 2 and 3 (HPIV1, 2 and 3), which collectively are the second leading cause of pediatric respiratory hospitalizations following respiratory syncytial virus (RSV) (Karron, R. A., and P. L. Collins. 2007. Parainfluenza Viruses, p. 1497-1526. In D. M. Knipe, P. M. Howley, D. E. Griffin, R. A. Lamb, M. A. Martin, B. Roizman, and S. E. Strauss (ed.), Fields Virology, 5th ed, vol. 1. Lippincott Williams & Wilkins, Philadelphia; Murphy, B. R., G. A. Prince, P. L. Collins, K. Van Wyke Coelingh, R. A. Olmsted, M. K. Spriggs, R. H. Parrott, H. W. Kim, C. D. Brandt, and R. M. Chanock. 1988. Current approaches to the development of vaccines effective against parainfluenza and respiratory syncytial viruses. Virus Res 11:1-15.) HPIV1 is responsible for approximately 6% of pediatric hospitalizations due to respiratory tract disease. Clinical manifestations range from mild disease, including rhinitis, pharyngitis, and otitis media, to more severe disease, including croup, bronchiolitis, and pneumonia. The HPIV1 genome is 15,600 nucleotides in length and contains six genes in the order 3′-N-P/C-M-F-HN-L-5′ (Newman, J. T., S. R. Surman, J. M. Riggs, C. T. Hansen, P. L. Collins, B. R. Murphy, and M. H. Skiadopoulos. 2002. Sequence analysis of the Washington/1964 strain of human parainfluenza virus type 1 (HPIV1) and recovery and characterization of wild-type recombinant HPIV1 produced by reverse genetics. Virus Genes 24:77-92.). Each gene encodes a single protein with the exception of the P/C gene that encodes the phosphoprotein, P, in one open reading frame (ORF) and up to four accessory C proteins, C′, C, Y1 and Y2, in a second ORF. The C proteins initiate at four separate translational start codons in the C ORF in the order C′, C, Y1, and Y2 and are carboxy co-terminal (Karron, R. A., and P. L. Collins. 2007. Parainfluenza Viruses, p. 1497-1526. In D. M. Knipe, P. M. Howley, D. E. Griffin, R. A. Lamb, M. A. Martin, B. Roizman, and S. E. Strauss (ed.), Fields Virology, 5th ed, vol. 1. Lippincott Williams & Wilkins, Philadelphia.), though it is unclear whether the Y2 protein is actually expressed during HPIV1 infection (Power, U. F., K. W. Ryan, and A. Portner. 1992. The P genes of human parainfluenza virus type 1 clinical isolates are polycistronic and microheterogeneous. Virology 189:340-3.). C proteins are expressed by viruses of the Respirovirus, Morbillivirus and Henipahvirus genera but not by viruses of the Rubulavirus and Avulavirus genera. The C proteins of Sendai virus (SeV), a member of the Respirovirus genus and the closest homolog of HPIV1, are perhaps the most extensively studied and have been shown to have multiple functions, including inhibition of the host innate immune response by acting as interferon (IFN) antagonists (Garcin, D., J. B. Marq, S. Goodbourn, and D. Kolakofsky. 2003. The amino-terminal extensions of the longer Sendai virus C proteins modulate pY701-Stat1 and bulk Stat1 levels independently of interferon signaling. J Virol 77:2321-9; Gotoh, B., K. Takeuchi, T. Komatsu, and J. Yokoo. 2003. The STAT2 activation process is a crucial target of Sendai virus C protein for the blockade of alpha interferon signaling. J Virol 77:3360-70; Komatsu, T., K. Takeuchi, J. Yokoo, and B. Gotoh. 2004. C and V proteins of Sendai virus target signaling pathways leading to IRF-3 activation for the negative regulation of interferon-beta production. Virology 325:137-48.).

To date, the HPIV1 C proteins have not been extensively studied, although recent studies provide evidence for a role for these proteins in the evasion of the host innate immune response. In A549 cells, a human lung adenocarcinoma epithelial cell line, it has previously been shown that type I IFN production was not detected during infection with HPIV1 wild type (wt). Since type I IFN was induced during infection of A549 cells with a recombinant HPIV1 (rHPIV1) mutant with C proteins bearing a F170S amino acid substitution, rHPIV1-C^{F170S}, a role for the C proteins as antagonists of the type I IFN response was suggested. This function was demonstrated to affect the innate immune response at the level of type I IFN induction and IFN signaling (Van Cleve, W., E. Amaro-Carambot, S. R. Surman, J. Bekisz, P. L. Collins, K. C. Zoon, B. R. Murphy, M. H. Skiadopoulos, and E. J. Bartlett. 2006. Attenuating mutations in the P/C gene of human parainfluenza virus type 1 (HPIV1) vaccine candidates abrogate the inhibition of both induction and signaling of type I interferon (IFN) by wild-type HPIV1. Virology 352:61-73.). Another study independently confirmed the role for the HPIV1 C proteins as antagonists of the type I IFN response, demonstrating that HPIV1 infection could inhibit STAT1 nuclear translocation and overcome an established IFN-induced antiviral state in MRC-5 human lung fibroblast cells, and furthermore that HPIV1 C protein expression was sufficient to inhibit STAT1 nuclear translocation in A549 cells. In contrast to the first study, the latter study demonstrated that type I IFN was induced during infection of MRC-5 cells with HPIV1 wt (Bousse, T., R. L. Chambers, R. A. Scroggs, A. Portner, and T. Takimoto. 2006. Human parainfluenza virus type 1 but not Sendai virus replicates in human respiratory cells despite IFN treatment. Virus Res 121:23-32.), which suggests that inhibition of type I IFN induction is cell-type specific. Therefore, it would be desirable to better define the host IFN response in relevant cell-types that are infected during HPIV1 infection in humans.

In vitro models of human airway epithelium (HAE) that closely mimic the morphological and physiological features of the human airway epithelium in vivo are now well characterized (Zhang, L., M. E. Peeples, R. C. Boucher, P. L. Collins, and R. J. Pickles. 2002. Respiratory syncytial virus infection of human airway epithelial cells is polarized, specific to ciliated cells, and without obvious cytopathology. J Virol 76:5654-66.). These models use freshly isolated human airway cells grown at an air-liquid interface (ALI) to generate a differentiated, pseudo-stratified, mucociliary epithelium that bears close structural and functional similarity to human airway epithelium in vivo. Such models have previously been used to demonstrate that paramyxoviruses such as RSV and HPIV3 preferentially infect ciliated cells, suggesting that these cells play a critical role in paramyxovirus replication and pathogenesis in the respiratory tract (Zhang, L., A. Bukreyev, C. I. Thompson, B. Watson, M. E. Peeples, P. L. Collins, and R. J. Pickles. 2005. Infection of ciliated cells by human parainfluenza virus type 3 in an in vitro model of human airway epithelium. J Virol 79:1113-24; Zhang, L., M. E. Peeples, R. C. Boucher, P. L. Collins, and R. J. Pickles. 2002. Respiratory syncytial virus infection of human airway epithelial cells is polarized, specific to ciliated cells, and without obvious cytopathology. J Virol 76:5654-66.). In addition, HAE models have been used to evaluate the attenuation of RSV vaccines (Wright, P. F., M. R. Ikizler, R. A. Gonzales, K. N. Carroll, J. E. Johnson, and J. A. Werkhaven. 2005. Growth of respiratory syncytial virus in primary epithelial cells from the human respiratory tract. J Virol 79:8651-4.).

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1. Comparison of the replication of HPIV1 wt and rHPIV1 mutant viruses containing the indicated mutations in the P/C, HN and L genes in a multiple cycle growth curve in LLC-MK2 and Vero cells (MOI=0.01).

FIG. 2. Representation of the association between the in vitro shut-off temperature and the attenuation phenotype in AGMs for HPIV1 wt (W) and rHPIV1 mutant viruses. The numbers refer to the following viruses: 2) rHPIV1-C^{R84G}; 3) rHPIV1-C^{R84G}HN^{T553A}; 4) rHPIV1-C^{Δ170}; 5) rHPIV1-L^{Y942A}; 6) rHPIV1-C^{R84G}HN^{T553A}L^{Y942A}; 7) rHPIV1-C^{R84G}L^{Δ1710-11}; 8) rHPIV1-C^{R84G/Δ170}L^{Y942A}; 9) rHPIV1-C^{R84G/Δ170}HN^{T553A}L^{Δ1710-11}.

FIG. 3. Representation of the relationship between the level of replication of HPIV1 wt and rHPIV1 mutants in AGMs and the subsequent level of replication of HPIV1 wt challenge virus in the immunized animals.

FIG. 4. rHPIV1 wt infects HAE cells, spreads throughout the culture and replicates efficiently.

FIG. 5. Comparison of single cycle virus growth curves in HAE inoculated with rHPIV1 wt (A) or rHPIV1-C^{F170S} (B) at an MOI of 5.0 TCID₅₀/cell or with VSV (C) at an MOI of 4.2 PFU/cell, at 37° C.

FIG. 6. HPIV1 infection of ciliated cells without overt cytotoxicity; immunofluorescence (A) or H&E staining (B) at 40× magnification or stained en face (C). For histological immunofluorescence, antibodies to HPIV1 (green) and alpha-acetylated tubulin (red) were used to detect virus antigen and ciliated cells, respectively. Scale bars represent 20 μm (A and B) and 40 μm (C).

FIG. 7. Comparison of the type I IFN response in HAE inoculated with rHPIV1 wt and rHPIV1-C^{F170S}; apical compartment (A), basolateral compartment (B).

FIG. 8. Virus replication (line graph) and type I IFN production (bar graph) during multi-cycle growth curves in HAE inoculated with rHPIV1 wt and rHPIV1-C^{F170S} at an MOI of 0.01 TCID₅₀/cell at 37° C. The area shaded in gray represents the overall difference in virus replication between rHPIV1 wt and rHPIV1-C^{F170S} after day 2 p.i.

FIG. 9. The ability of HPIV1 vaccine candidates to replicate in HAE at 32° C. and 37° C. was determined by multi-cycle growth curves (MOI=0.01).

FIG. 10. Designing the HPIV1-P(C-) viral cDNA. (A) The HPIV1 wt genome, shown 3′ to 5′, includes the P/C gene that encodes the phosphoprotein P from one ORF and the four carboxy-coterminal C proteins, C′, C, Y1 and Y2, from a second, overlapping ORF. The coding regions for these proteins are shown, with the initiation and termination codons numbered according to the P/C gene sequence. (B) Various mutations were introduced into the HPIV1 P/C gene to silence expression of the four C proteins without affecting the amino acid sequence of the P protein. Panel B shows the sequence of the upstream end of the P/C gene, with the transcription gene-start signal and the translational start signal for each protein boxed. Nucleotide (nt) substitutions and an insertion in the rHPIV1-P(C-) sequence are indicated in boldface, and a deletion is indicated with a dotted line. These mutations are identified with circled numbers that correspond with a description in panel (C) of the effect of each mutation. Briefly, 93 nt were deleted between the gene-start signal and P start codon and replaced with a 6-nt spacer CCCAAC (mutations 1 and 2), thus eliminating the first 11 codons of C′ including its start codon. The sequence immediately upstream of the P start codon was modified: CGA(ATG) to AAC(ATG), which will also optimize the Kozak sequence and reduce translational initiation at the downstream start codons (mutation 1). The methionine start codon of the C protein was converted to threonine (mutation 3), and one stop codon was introduced downstream of the Y1 start codon (mutation 4) and two stop codons were introduced downstream of the Y2 start codon (mutations 5 and 6). This cDNA was used to recover infectious rHPIV1-P(C-).

FIG. 11. Identification of HPIV1 C and P proteins in lysates from infected LLC-MK2 cells. Lysates were prepared 48 h p.i. from LLC-MK2 cells that were mock-infected or infected with sucrose-purified rHPIV1 wt or rHPIV1-P(C-) at an input MOI of 5 TCID₅₀/cell. Reduced, denatured cell lysates were resolved by SDS-PAGE electrophoresis and Western blots were prepared and analyzed using rabbit anti-peptide antisera against (A) the HPIV1 C proteins and (B) the HPIV1 P protein. The asterisk (*) in panel A indicates a new band of unknown identity, detected only in the rHPIV1-P(C-)-infected cell lysates.

FIG. 12. Comparison of the replication of rHPIV1 wt and rHPIV1-P(C-) viruses in vitro. (A) Multi-cycle replication in LLC-MK2 cells infected at a MOI of 0.01 TCID₅₀/cell. On days 0-7 p.i., the overlying medium was harvested for virus titration, shown as the means of 3 replicate cultures. On days 1-7 p.i., the cell monolayers were monitored for cpe and assigned a score of 1-5 according to the extent of cpe (Materials and Methods), shown as the means of the 3 replicate cultures. (B) LLC-MK2 cells were mock-infected or infected with rHPIV1 wt or rHPIV1-P(C-) at a MOI of 0.01 or 5 TCID₅₀/cell, as indicated in parentheses below the virus names. Photomicrographs taken at 72 h p.i. show increased cytopathic effect (cpe) in the rHPIV1-P(C-)-infected cultures (magnification, ×10).

FIG. 13. Infection with rHPIV1-P(C-) induces activation of caspase 3, indicative of apoptosis. Caspase 3 activation was evaluated by immunostaining and FACS analysis. (A) Evaluation of caspase 3 activation by immunofluorescence. LLC-MK2 cells were mock-infected or infected with rHPIV1 wt or rHIV1-P(C-) at a MOI of 10 TCID₅₀/cell. At 72 h p.i., cells were fixed, permeabilized, and stained for HPIV1 HN protein (red) and activated caspase 3 (green), and nuclei were stained with DAPI (blue). Cells were visualized by confocal microscopy and scale bars represent 10 μm. (B) Evaluation of caspase 3 activation by FACS analysis. LLC-MK2 cells were mock-infected or infected with rHPIV1 wt, rHPIV1-C^{F170S}, or rHPIV1-P(C-) at a MOI of 5 TCID₅₀/cell in triplicate. Cells were harvested at 24, 48, and 72 h p.i., fixed, permeabilized and stained for HPIV1 HN and activated caspase 3 in FACS buffer prior to analysis. Sample analysis was carried out using a FACSCalibur flow cytometer and FlowJo software. Dot plots of representative data for samples from the 48 h time point are shown. (C) Percentage of cells positive for activated caspase 3 at 24, 48 and 72 h p.i., as determined by FACS analysis±S.E.

FIG. 14. rHPIV1 wt, but not rHPIV1-P(C-), inhibits type I IFN induction and signaling. (A) Induction of type I IFN. A549 cell monolayers were either mock-infected or infected with rHPIV1 wt, rHPIV1-C^{F170S}, or rHPIV1-P(C-) at a MOI of 5 TCID₅₀/cell. Aliquots of the overlying medium were taken at 0, 24, 48 and 72 h p.i. and assayed on fresh cells for the ability to inhibit infection and GFP expression by VSV-GFP as measured with a phosphorimager. IFN concentrations were determined by comparison with a standard curve prepared in parallel with an AVONEX® IFN-β standard and are expressed in pg/ml±SE based on triplicate samples. The lower limit of detection was 39.1 pg/ml (dashed line). (B) Type I IFN signaling. Vero cells in 6-well plates were infected with the indicated rHPIV1s at a MOI of 5 TCID₅₀/cell and incubated for 24 h. Cells were then left untreated or were treated with 100 or 1000 IU/ml IFN-β (1 well per treatment per virus) for 24 h. The cells were then infected with VSV-GFP and incubated for 48 h. The VSV-GFP foci were visualized using a phosphorimager and counted. The graph represents the percent inhibition of VSV-GFP replication in IFN-β treated versus untreated cells based on two independent experiments.

FIG. 15. rHPIV1-P(C-) is attenuated for replication in both the URT and LRT of AGMs. Groups of AGMs were inoculated i.n. and i.t. with 10⁶ TCID₅₀ of HPIV1 wt (n=16) or rHPIV1-P(C-) (n=4) per site. Previously published data for rHPIV1-C^{F170S} (n=4) also are included for comparison (Bartlett, E. J., E. Amaro-Carambot, S. R. Surman, J. T. Newman, P. L. Collins, B. R. Murphy, and M. H. Skiadopoulos. 2005. Human parainfluenza virus type I (HPIV1) vaccine candidates designed by reverse genetics are attenuated and efficacious in African green monkeys. Vaccine 23:4631-46). Mean daily virus titers±SE were determined in (A) nasopharyngeal (NP) swabs (representative of the URT) and (B) tracheal lavage (TL) fluid (representative of the LRT) for each sampling day (see Materials and Methods; the limit of detection is 0.5 log₁₀ TCID₅₀/ml). The area shaded in grey represents the additional reduction in replication observed for rHPIV1-P(C-) compared to rHPIV1-C^{F170S}.

FIG. 16. rHPIV1-P(C-) replicates very poorly in primary human airway epithelial (HAE) cells compared to rHPIV1 wt. HAE cultures were inoculated on the apical surface with either virus at a MOI of 0.01 TCID₅₀/cell, and virus titers were determined in apical surface washes at days 0-7 p.i. These are shown as the means of triplicate cultures from two donors±S.E., and the limit of detection is 1.2 log₁₀TCID₅₀/ml.

### DETAILED DESCRIPTION OF THE INVENTION

This application describes an infectious, recombinant, self-replicating attenuated human parainfluenza virus type 1 (HPIV1) particle that is a complete or partial particle. In some embodiments, the minimal particle is made up of a nucleocapsid protein (N), a nucleocapsid phosphoprotein (P), a large polymerase protein (L), a C protein and a HN glycoprotein. These proteins are encoded by a partial or complete genome or antigenome.

In another embodiment of the invention, the infectious, recombinant, self-replicating attenuated human parainfluenza virus type 1 (HPIV1) particle is a complete or partial particle such that the minimal particle is made up of a nucleocapsid protein (N), a nucleocapsid phosphoprotein (P), and a large polymerase protein (L). These proteins are encoded by a partial or complete genome or antigenome that maintains a “wild type” gene structure of overlapping C and P open reading frames, described below, but while the P protein is expressed, none of the C proteins are expressed. An example of such an embodiment is the HPIV1 P(C-) virus described in detail in Example 3.

This application further describes particular mutations in the C protein, such as a mutation in the codon encoding amino acid R84 in the C protein that gives rise to a different amino acid, for example glycine, or a deletion in the codon encoding amino acid 170 of the C protein. Mutation of the P/C gene such that it expresses a P protein, and particularly a wild type P protein, but does not express any of the C proteins encoded in the P/C gene is also contemplated. Similarly, the application describes particular mutations in the HN glycoprotein, such as a mutation in the codon encoding amino acid T553 which gives rise to a different amino acid, for example alanine. This application also describes mutations in the L protein, such as a mutation in the codon encoding amino acid Y942 that gives rise to a different amino acid, for example alanine, or a deletion of particular codons, for example those encoding amino acids 1710 and 1711.

The complete sequences of two HPIV1 vaccine candidate viruses according to the invention, designated as rHPIV1-C^{R84G/Δ170}HN^{T553A}L^{Y942A} and rHPIV1-C^{R84G/Δ170}HN^{T553A}L^{Δ1710-11} are appended hereto as SEQ ID NO: 1 and SEQ ID NO: 2, respectively. The complete sequence of another vaccine candidate virus HPIV1 P(C-) is presented as SEQ ID NO: 5. These sequences are antigenome sequences and are presented in the 5′ to 3′ direction. Thus, the 3′ end of SEQ ID NOS: 1, 2 and 5, as presented, represents the 3′ leader of the nucleic acid as packaged in the viral particle.

This application also describes a polynucleotide encoding the genome or antigenome of an infectious, recombinant, self-replicating attenuated human parainfluenza virus type 1 (HPIV1) particle encoded by a partial or complete genome or antigenome. In some embodiments of the invention, the genome or antigenome includes a polynucleotide, gene or genome segment of an antigenic determinant of a non-HPIV1 pathogen or a polynucleotide encoding a host cell immune regulatory protein.

The infectious, recombinant, self-replicating attenuated human parainfluenza virus type 1 (HPIV1) particles of the invention that contain exogenous antigenic determinants can include at least non-HPIV1 determinants from a glycoprotein of a HPIV2, HPIV3, RSV, measles virus, influenza virus, or other non-HPIV1 pathogen.

The infectious, recombinant, self-replicating attenuated HPIV1 particles of the invention can also include genes that encode various immune system modulatory molecules. Such genes may encode a cytokine, chemokine, enzyme, cytokine antagonist, chemokine antagonist, surface receptor, soluble receptor, adhesion molecule, or ligand. Preferred immune modulatory genes for use in the present invention include those that encode interleukin 2 (IL-2), interleukin 4 (IL-4), interferon gamma (IFNγ) and granulocyte-macrophage colony stimulating factor (GM-CSF).

This application further describes an expression vector which has a promoter which functions in a mammalian cell or in a cell free system operatively linked to a polynucleotide encoding the genome or antigenome of an infectious, recombinant, self-replicating attenuated human parainfluenza virus type 1 (HPIV1) particle and that is in turn operatively linked to a transcription terminator which also functions in a mammalian cell or in a cell free system. Host cells that contain the expression vector are also described. Such host cells might also include one or more additional expression vectors that express a PIV N, P, or L protein, or a combination of those proteins.

In addition, this application describes a method for producing an infectious, recombinant, self-replicating attenuated HPIV1. Briefly, a nucleocapsid protein (N), a nucleocapsid phosphoprotein (P) and a large polymerase protein (L) of a human parainfluenza virus is expressed in a host cell that also contains a polynucleotide encoding the genome or antigenome of an infectious, recombinant, self-replicating attenuated human parainfluenza virus type 1 (HPIV1) particle. For example the host cell produces infectious viral particle comprising N, P and L proteins and a partial or complete genome or antigenome encoding at least a nucleocapsid protein (N), a nucleocapsid phosphoprotein (P), a large polymerase protein (L), a C protein and a HN glycoprotein. In some embodiments the partial or complete genome or antigenome encodes a C protein with a mutation in the codon encoding amino acid R84 that substitutes another amino acid or that has a deletion in the codon encoding amino acid 170. In some embodiments, the partial or complete genome or antigenome expresses a P protein, but does not express any of the proteins encoded by the C gene. In some embodiments the partial or complete genome or antigenome encodes an HN glycoprotein with a mutation in the codon encoding amino acid T553 such that another amino acid is substituted. In yet other embodiments the partial or complete genome or antigenome encodes an L protein that has a mutation in the codon encoding amino acid Y942 which encodes a different amino acid or that has a deletion of the codons encoding amino acids 1710 and 1711. In some embodiments the N, P and L proteins are expressed from one or more than one expression vector.

This application also describes an immunogenic composition comprising the HPIV1 particle described above, with and without a pharmaceutically acceptable excipient or carrier. An immunogenic composition that is formulated at a titer of 10³ to 10⁶ pfu/ml in the form of an aerosol or intranasal spray or droplet is also described.

The instant invention provides methods and compositions for the production and use of novel human parainfluenza virus type 1 (HPIV1) candidates for use in immunogenic compositions. The recombinant HPIV1 viruses of the invention are infectious and immunogenic in humans and other mammals and are useful for generating immune responses against one or more PIVs, for example against one or more human PIVs (HPIVs). In additional embodiments, chimeric HPIV1 viruses are provided that elicit an immune response against a selected PIV and one or more additional pathogens, for example against multiple HPIVs or against a HPIV and a non-PIV virus such as respiratory syncytial virus (RSV), human metapneumovirus, or measles virus. The immune response elicited can involve either or both humoral and/or cell mediated responses. Preferably, recombinant HPIV1 viruses of the invention are attenuated to yield a desired balance of attenuation and immunogenicity for use in immunogenic compositions. The invention thus provides novel methods for designing and producing attenuated, HPIV1 viruses that are useful as immunological agents to elicit immune responses against HPIV1 and other pathogens.

Exemplary recombinant HPIV1 viruses of the invention incorporate a recombinant HPIV1 genome or antigenome, as well as a PIV major nucleocapsid (N) protein, a nucleocapsid phosphoprotein (P), and a large polymerase protein (L). The N, P, and L proteins may be HPIV1 proteins, or one or more of the N, P, and L proteins may be of a different HPIV, for example HPIV3. Additional PIV proteins may be included in various combinations to provide a range of infectious viruses, defined herein to include subviral particles lacking one or more non-essential viral components and complete viruses having all native viral components, as well as viruses containing supernumerary proteins, antigenic determinants or other additional components.

A complete consensus sequence (GenBank Accession No. AF457102, incorporated herein by reference) was determined herein for the genomic RNA of a multiply-passaged human parainfluenza virus type 1 (HPIV1) strain (designated HPIV1_{LLC4}) derived from a wild-type (wt) clinical isolate Washington/20993/1964 that has been shown to be virulent in adults (Murphy et al., Infect. Immun. 12:62-68, 1975, incorporated herein by reference). The sequence thus identified exhibits a high degree of relatedness to both Sendai virus (a PIV1 virus isolated from mice that is referred to here as MPIV1), and human PIV3 (HPIV3) with regard to cis-acting regulatory regions and protein-coding sequences. This consensus sequence was used to generate a full-length antigenomic cDNA and to recover a recombinant wild-type HPIV1 (rHPIV1). The rHPIV1 could be rescued from full-length antigenomic rHPIV1 cDNA using HPIV3 support plasmids, HPIV1 support plasmids, or a mixture thereof.

The replication of rHPIV1 in vitro and in the respiratory tract of hamsters was similar to that of its biologically derived parent virus. The similar biological properties of rHPIV1 and HPIV1 WASH/64 in vitro and in vivo, together with the previous demonstration of the virulence of this specific isolate in humans, authenticates the rHPIV1 sequence as that of a wild-type virus. This is a critical finding since the high mutation rate characteristic of these viruses often results in errors that reduce viability. This rHPIV1 therefore serves as a novel and proven substrate for recombinant introduction of attenuating mutations for the generation of live-attenuated HPIV1 recombinants.

The Paramyxovirinae subfamily of the Paramyxoviridae family of viruses includes human parainfluenza virus types 1, 2, 3, 4A and 4B (HPIV1, HPIV2, HPIV3, HPIV4A, and HPIV4B, respectively). HPIV1, HPIV3, MPIV1, and bovine PIV3 (BPIV3) are classified together in the genus Respirovirus, whereas HPIV2 and HPIV4 are more distantly related and are classified in the genus Rubulavirus. MPIV1, simian virus 5 (SV5), and BPIV3 are animal counterparts of HPIV1, HPIV2, and HPIV3, respectively (Chanock et al., in Parainfluenza Viruses, Knipe et al. (Eds.), pp. 1341-1379, Lippincott Williams & Wilkins, Philadelphia, 2001, incorporated herein by reference).

The human PIVs have a similar genomic organization, although significant differences occur in the P gene (Chanock et al., in Parainfluenza Viruses, Knipe et al. (eds.), pp. 1341-1379, Lippincott Williams & Wilkins, Philadelphia, 2001; Lamb et al., in Paramyxoviridae: The viruses and their replication, Knipe et al. (eds.), pp. 1305-1340, Lippincott Williams & Wilkins, Philadelphia, 2001, each incorporated herein by reference). The 3′ end of genomic RNA and its full-length, positive-sense replicative intermediate antigenomic RNA contain promoter elements that direct transcription and replication. The nucleocapsid-associated proteins are composed of the nucleocapsid protein (N), the phosphoprotein (P), and the large polymerase (L). The internal matrix protein (M) and the major antigenic determinants, the fusion glycoprotein (F) and hemagglutinin-neuraminidase glycoprotein (HN) are the envelope-associated proteins. The gene order is N, P, M, F, HN, and L.

With the exception of the P gene, each HPIV gene contains a single ORF and encodes a single viral protein. The P gene of the Paramyxovirinae subfamily encodes a number of proteins that are generated from alternative open reading frames (ORFs), by the use of alternative translational start sites within the same ORF, by an RNA polymerase editing mechanism, by ribosomal shunting, or through ribosomal frame shifting (Lamb et al., in Paramyxoviridae: The viruses and their replication, Knipe et al. (Eds.), pp. 1305-1340, Lippincott Williams & Wilkins, Philadelphia, 2001; Liston et al., J Virol 69:6742-6750, 1995; Latorre et al., Mol. Cell. Biol. 18:5021-5031, 1998, incorporated herein by reference). For example, the MPIV1 P gene expresses eight proteins. Four of these, C, C′, Y1, and Y2, are expressed by translational initiation at four different codons within the C ORF that is present in a +1 reading frame relative to the P ORF (Curran et al., Embo J. 7:245-251, 1988, Dillon et al., J. Virol. 63:974-977, 1989; Curran et al., Virology 189:647-656, 1989, each, incorporated herein by reference).

In HPIV1, the translation start sites for the C′, C, Y1, and Y2 proteins are, respectively, a nonstandard GUG codon at nucleotides (nt) 69-71 (numbered according to the P mRNA), AUG codons at nt 114-117, 183-185, and a nonstandard ACG at nt 201-203 (for comparison, the translation start site for the P ORF is at nt 104-106) (Curran et al., Embo J. 7:245-251, 1988, incorporated herein by reference). Expression of the Y1 and Y2 proteins involves a ribosomal shunt mechanism (Latorre et al., Mol Cell Biol 18:5021-5031, 1998, incorporated herein by reference). MPIV1 also expresses this set of proteins, which collectively act to down regulate viral replication, contribute to virion assembly, and interfere with interferon action (Curran et al., Virology 189:647-656, 1992; Tapparel et al., J. Virol. 71:9588-9599, 1997; Garcin et al., J. Virol. 74:8823-8830, 2000; Hasan et al., J. Virol. 74:5619-5628, 2000; Garcin et al., J. Virol. 75:6800-6807, 2001; Kato et al., J. Virol. 75:3802-3810, 2001, each incorporated herein by reference). See also, Bartlett, E. J., E. Amaro-Carambot, S. R. Surman, J. T. Newman, P. L. Collins, B. R. Murphy, and M. H. Skiadopoulos. 2005. Human parainfluenza virus type I (HPIV1) vaccine candidates designed by reverse genetics are attenuated and efficacious in African green monkeys. Vaccine 23:4631-46. This reference includes a figure illustrating the positions of the start sites of HPIV1 C proteins.

The MPIV1 P ORF gives rise to the P protein and to two additional proteins, V and W, which share the N-terminal half of the P protein but which each have a unique carboxy-terminus due an RNA polymerase-dependent editing mechanism that inserts one or two G residues, respectively (Curran et al., Embo J. 10:3079-3085, 1991, incorporated herein by reference). In W, the carboxy-terminal extension that results from the frame shift consists of a single added amino acid, while that of V contains a cysteine-rich domain that is highly conserved among members of Paramyxovirinae (Lamb et al., in Paramyxoviridae: The viruses and their replication, Knipe et al. (Eds.), pp. 1305-1340, Lippincott Williams & Wilkins, Philadelphia, 2001, incorporated herein by reference). The V protein does not appear to be necessary for MPIV1 replication in cell culture, but mutants that lack this protein are attenuated in mice (Kato et al., EMBO J. 16:578-587, 1997, incorporated herein by reference).

In MPIV1, an additional protein, X, is expressed from the downstream end of the MPIV1 P ORF by a mode of translational initiation that appears to be dependent on the 5′ cap but is independent of ribosomal scanning (Curran et al., Embo J. 7:2869-2874, 1988, incorporated herein by reference). As another example, measles virus encodes a P protein, a V protein, a single C protein, and a novel R protein (Liston et al., J. Virol. 69:6742-6750, 1995; Bellini et al., J. Virol. 53:908-919, 1985; Cattaneo et al., Cell 56, 759-764, 1989, each incorporated herein by reference). R is a truncated version of P attached to the downstream end of V, and likely results from a ribosomal frame shift during translation of the downstream half of the P ORF (Liston et al., J Virol 69:6742-6750, 1995, incorporated herein by reference). For HPIV1, in vitro translation experiments suggest the expression of C′, C, and Y1 proteins (Power et al., Virology 189:340-343, 1992, incorporated herein by reference). HPIV1 encodes a P protein but does not appear to encode a V protein, based on the lack of a homologous RNA editing site and the presence of a relict V coding sequence that is interrupted by 9-11 stop codons (Matsuoka et al., J. Virol. 65:3406-3410, 1991; Rochat et al., Virus Res. 24:137-144, 1992, incorporated herein by reference).

Infectious recombinant HPIV1 viruses according to the invention are produced by a recombinant coexpression system that permits introduction of defined changes into the recombinant HPIV1. These modifications are useful in a wide variety of applications, including the development of live attenuated HPIV1 strains bearing predetermined, defined attenuating mutations. Infectious PIV of the invention are typically produced by intracellular or cell-free coexpression of one or more isolated polynucleotide molecules that encode the HPIV1 genome or antigenome RNA, together with one or more polynucleotides encoding the viral proteins desired, or at least necessary, to generate a transcribing, replicating nucleocapsid.

cDNAs encoding a HPIV1 genome or antigenome are constructed for intracellular or in vitro coexpression with the selected viral proteins to form infectious PIV. By “HPIV antigenome” is meant an isolated positive-sense polynucleotide molecule which serves as a template for synthesis of progeny HPIV genomes. Preferably a cDNA is constructed which is a positive-sense version of the HPIV genome corresponding to the replicative intermediate RNA, or antigenome, so as to minimize the possibility of hybridizing with positive-sense transcripts of complementing sequences encoding proteins necessary to generate a transcribing, replicating nucleocapsid.

In some embodiments of the invention the genome or antigenome of a recombinant HPIV (rHPIV) need only contain those genes or portions thereof necessary to render the viral or subviral particles encoded thereby infectious. Further, the genes or portions thereof may be provided by more than one polynucleotide molecule, i.e., a gene may be provided by complementation or the like from a separate nucleotide molecule. In other embodiments, the PIV genome or antigenome encodes all functions necessary for viral growth, replication, and infection without the participation of a helper virus or viral function provided by a plasmid or helper cell line.

By “recombinant HPIV” (including recombinant HPIV1) is meant a HPIV or HPIV-like viral or subviral particle derived directly or indirectly from a recombinant expression system or propagated from virus or subviral particles produced therefrom. The recombinant expression system will employ a recombinant expression vector which comprises an operably linked transcriptional unit comprising an assembly of at least a genetic element or elements having a regulatory role in PIV gene expression, for example, a promoter, a structural or coding sequence which is transcribed into PIV RNA, and appropriate transcription initiation and termination sequences.

To produce infectious HPIV from a cDNA-expressed HPIV genome or antigenome, the genome or antigenome is coexpressed with those PIV (HPIV1 or heterologous PIV) proteins necessary to produce a nucleocapsid capable of RNA replication, and render progeny nucleocapsids competent for both RNA replication and transcription. Transcription by the genome nucleocapsid provides the other PIV proteins and initiates a productive infection. Alternatively, additional PIV proteins needed for a productive infection can be supplied by coexpression.

Synthesis of a HPIV particle comprising a antigenome or genome together with the above-mentioned viral proteins can also be achieved in vitro (cell-free), e.g., using a combined transcription-translation reaction, followed by transfection into cells. Alternatively, antigenome or genome RNA can be synthesized in vitro and transfected into cells expressing PIV proteins. In certain embodiments of the invention, complementing sequences encoding proteins necessary to generate a transcribing, replicating HPIV nucleocapsid are provided by one or more helper viruses. Such helper viruses can be wild-type or mutant. Preferably, the helper virus can be distinguished phenotypically from the virus encoded by the HPIV cDNA. For example, it may be desirable to provide monoclonal antibodies which react immunologically with the helper virus but not the virus encoded by the HPIV cDNA. Such antibodies can be neutralizing antibodies. In some embodiments, the antibodies can be used in affinity chromatography to separate the helper virus from the recombinant virus. To aid the procurement of such antibodies, mutations can be introduced into the HPIV cDNA to provide antigenic diversity from the helper virus, such as in the HN or F glycoprotein genes.

Expression of the HPIV (including HPIV1) genome or antigenome and proteins from transfected plasmids can be achieved, for example, by each cDNA being under the control of a selected promoter (e.g., for T7 RNA polymerase), which in turn is supplied by infection, transfection or transduction with a suitable expression system (e.g., for the T7 RNA polymerase, such as a vaccinia virus MVA strain recombinant which expresses the T7 RNA polymerase, as described by Wyatt et al., Virology 210:202-205, 1995, incorporated herein by reference). The viral proteins, and/or T7 RNA polymerase, can also be provided by transformed mammalian cells or by transfection of preformed mRNA or protein.

A HPIV1 genome or antigenome may be constructed for use in the present invention by, e.g., assembling cloned cDNA segments, representing in aggregate the complete genome or antigenome, by polymerase chain reaction or the like (PCR; described in, e.g., U.S. Pat. Nos. 4,683,195 and 4,683,202, and PCR Protocols: A Guide to Methods and Applications, Innis et al., eds., Academic Press, San Diego, 1990, each incorporated herein by reference) of reverse-transcribed copies of HPIV1 mRNA or genome RNA. For example, a first construct may be generated which comprises cDNAs containing the left hand end of the antigenome, spanning from an appropriate promoter (e.g., T7 RNA polymerase promoter) and assembled in an appropriate expression vector, such as a plasmid, cosmid, phage, or DNA virus vector. The vector may be modified by mutagenesis and/or insertion of synthetic polylinker containing unique restriction sites designed to facilitate assembly. For ease of preparation the N, P, L and other desired PIV proteins can be assembled in one or more separate vectors. The right hand end of the antigenome plasmid may contain additional sequences as desired, such as a flanking ribozyme and tandem T7 transcriptional terminators. The ribozyme can be hammerhead type, which would yield a 3′ end containing a single nonviral nucleotide, or can be any of the other suitable ribozymes such as that of hepatitis delta virus (Perrotta et al., Nature 350:434-436, 1991, incorporated herein by reference) which would yield a 3′ end free of non-PIV nucleotides. The left- and right-hand ends are then joined via a common restriction site.

Alternative means to construct cDNA encoding an HPIV1 genome or antigenome include reverse transcription-PCR using improved PCR conditions (e.g., as described in Cheng et al., Proc. Natl. Acad. Sci. USA 91:5695-5699, 1994, incorporated herein by reference) to reduce the number of subunit cDNA components to as few as one or two pieces. In other embodiments different promoters can be used (e.g., T3, SPQ or different ribozymes (e.g., that of hepatitis delta virus. Different DNA vectors (e.g., cosmids) can be used for propagation to better accommodate the larger size genome or antigenome.

By “infectious clone” of HPIV (including HPIV1) is meant cDNA or its product, synthetic or otherwise, as well as RNA capable of being directly incorporated into infectious virions which can be transcribed into genomic or antigenomic HPIV RNA capable of serving as a template to produce the genome of infectious HPIV viral or subviral particles. As noted above, defined mutations can be introduced into an infectious HPIV clone by a variety of conventional techniques (e.g., site-directed mutagenesis) into a cDNA copy of the genome or antigenome. The use of genomic or antigenomic cDNA subfragments to assemble a complete genome or antigenome cDNA as described herein has the advantage that each region can be manipulated separately, where small cDNA constructs provide for better ease of manipulation than large cDNA constructs, and then readily assembled into a complete cDNA.

Isolated polynucleotides (e.g., cDNA) encoding the HPIV genome or antigenome may be inserted into appropriate host cells by transfection, electroporation, mechanical insertion, transduction or the like, into cells which are capable of supporting a productive HPIV (including HPIV1) infection, e.g., HEp-2, FRhL-DBS2, LLC-MK2, MRC-5, and Vero cells. Transfection of isolated polynucleotide sequences may be introduced into cultured cells by, for example, calcium phosphate-mediated transfection (Wigler et al., Cell 14:725, 1978; Corsaro et al., Somatic Cell Genetics 7:603, 1981; Graham et al., Virology 52:456, 1973, electroporation (Neumann et al., EMBO J. 1:841-845, 1982), DEAE-dextran mediated transfection (Ausubel et al., (ed.) Current Protocols in Molecular Biology, John Wiley and Sons, Inc., NY, 1987, cationic lipid-mediated transfection (Hawley-Nelson et al., Focus 15:73-79, 1993) or a commercially available transfection regent, e.g., Lipofectamine-2000 (Invitrogen, Carlsbad, Calif.) or the like (each of the foregoing references are incorporated herein by reference in its entirety).

By providing infectious clones of HPIV1, including the mutant viruses described herein, the invention permits a wide range of alterations to be recombinantly produced within the HPIV1 genome (or antigenome), yielding defined mutations that specify desired phenotypic changes. The compositions and methods of the invention for producing recombinant HPIV1 permit ready detailed analysis and manipulation of HPIV1 molecular biology and pathogenic mechanisms using, e.g., defined mutations to alter the function or expression of selected HPIV1 proteins. Using these methods and compositions, one can readily distinguish mutations responsible for desired phenotypic changes from silent incidental mutations, and select phenotype-specific mutations for incorporation into a recombinant HPIV1 genome or antigenome for production of immunogenic compositions. In this context, a variety of nucleotide insertions, deletions, substitutions, and rearrangements can be made in the HPIV1 genome or antigenome during or after construction of the cDNA. For example, specific desired nucleotide sequences can be synthesized and inserted at appropriate regions in the cDNA using convenient restriction enzyme sites. Alternatively, such techniques as site-specific mutagenesis, alanine scanning, PCR mutagenesis, or other such techniques well known in the art can be used to introduce mutations into the cDNA.

Recombinant modifications of HPIV1 provided within the invention are directed toward the production of improved viruses for use in immunogenic compositions, e.g., to enhance viral attenuation and immunogenicity, to ablate epitopes associated with undesirable immunopathology, to accommodate antigenic drift, etc. To achieve these and other objectives, the compositions and methods of the invention allow for a wide variety of modifications to be introduced into a HPIV1 genome or antigenome for incorporation into infectious, recombinant HPIV1. For example, foreign genes or gene segments encoding protective antigens or epitopes may be added within a HPIV1 clone to generate recombinant HPIV1 viruses capable of inducing immunity to both HPIV1 and another virus or pathogenic agent from which the protective antigen was derived. Alternatively, foreign genes may be inserted, in whole or in part, encoding modulators of the immune system, such as cytokines, to enhance immunogenicity of a candidate virus for use in immunogenic compositions. Other mutations which may be included within HPIV1 clones of the invention include, for example, substitution of heterologous genes or gene segments (e.g., a gene segment encoding a cytoplasmic tail of a glycoprotein gene) with a counterpart gene or gene segment in a PIV clone. Alternatively, the relative order of genes within a HPIV1 clone can be changed, a HPIV1 genome promoter or other regulatory element can be replaced with its antigenome counterpart, or selected HPIV1 gene(s) rendered non-functional (e.g., by functional ablation involving introduction of a stop codon to prevent expression of the gene). Other modifications in a HPIV1 clone can be made to facilitate manipulations, such as the insertion of unique restriction sites in various non-coding or coding regions of the HPIV1 genome or antigenome. In addition, nontranslated gene sequences can be removed to increase capacity for inserting foreign sequences.

As noted above, it is often desirable to adjust the phenotype of recombinant HPIV1 viruses for use in immunogenic compositions by introducing additional mutations that increase or decrease attenuation or otherwise alter the phenotype of the recombinant virus. Detailed descriptions of the materials and methods for producing recombinant PIV from cDNA, and for making and testing various mutations and nucleotide modifications set forth herein as supplemental aspects of the present invention are provided in, e.g., Durbin et al., Virology 235:323-332, 1997; U.S. patent application Ser. No. 09/083,793, filed May 22, 1998; U.S. patent application Ser. No. 09/458,813, filed Dec. 10, 1999; U.S. patent application Ser. No. 09/459,062, filed Dec. 10, 1999; U.S. Provisional Application No. 60/047,575, filed May 23, 1997 (corresponding to International Publication No. WO 98/53078), and U.S. Provisional Application No. 60/059,385, filed Sep. 19, 1997, each incorporated herein by reference.

In particular, these incorporated references describe methods and procedures for mutagenizing, isolating and characterizing PIV to obtain attenuated mutant strains (e.g., temperature sensitive (ts), cold passaged (cp) cold-adapted (ca), small plaque (sp) and host-range restricted (hr) mutant strains) and for identifying the genetic changes that specify the attenuated phenotype. In conjunction with these methods, the foregoing incorporated references detail procedures for determining replication, immunogenicity, genetic stability and immunogenic efficacy of biologically derived and recombinantly produced attenuated HPIVs in accepted model systems reasonably correlative of human activity, including hamster or rodent and non-human primate model systems. In addition, these references describe general methods for developing and testing immunogenic compositions, including monovalent and bivalent compositions, for eliciting an immune response against HPIV and other pathogens. Methods for producing infectious recombinant PIV by construction and expression of cDNA encoding a PIV genome or antigenome coexpressed with essential PIV proteins are also described in the above-incorporated references.

Also disclosed in the above-incorporated references are methods for constructing and evaluating infectious recombinant HPIV that are modified to incorporate phenotype-specific mutations identified in biologically derived PIV mutants, e.g., cold passaged (cp), cold adapted (ca), host range restricted (hr), small plaque (sp), and/or temperature sensitive (ts) mutants, for example the JS HPIV3 cp45 mutant strain. The HPIV3 JS cp45 strain has been deposited under the terms of the Budapest Treaty with the American Type Culture Collection (ATCC) of 10801 University Boulevard, Manassas, Va. 20110-2209, U.S.A. under Patent Deposit Designation PTA-2419 (deposit incorporated herein by reference). Mutations identified in this and other heterologous mutants viruses can be readily incorporated into recombinant HPIV1 of the instant invention, as described previously.

Nucleotide modifications that may be introduced into recombinant HPIV1 constructs of the invention may alter small numbers of bases (e.g., from 15-30 bases, up to 35-50 bases or more), large blocks of nucleotides (e.g., 50-100, 100-300, 300-500, 500-1,000 bases), or nearly complete or complete genes (e.g., 1,000-1,500 nucleotides, 1,500-2,500 nucleotides, 2,500-5,000, nucleotides, 5,00-6,5000 nucleotides or more) in the vector genome or antigenome or heterologous, donor gene or genome segment, depending upon the nature of the change (i.e., a small number of bases may be changed to insert or ablate an immunogenic epitope or change a small genome segment, whereas large block(s) of bases are involved when genes or large genome segments are added, substituted, deleted or rearranged).

In related aspects, the invention provides for supplementation of mutations adopted into a recombinant HPIV1 clone from biologically derived PIV, e.g., cp and ts mutations, with additional types of mutations involving the same or different genes in a further modified recombinant HPIV1. Each of the HPIV1 genes can be selectively altered in terms of expression levels, or can be added, deleted, substituted or rearranged, in whole or in part, alone or in combination with other desired modifications, to yield a recombinant HPIV1 exhibiting novel immunological characteristics. Thus, in addition to or in combination with attenuating mutations adopted from biologically derived PIV and/or non-PIV mutants, the present invention also provides a range of additional methods for attenuating or otherwise modifying the phenotype of a recombinant HPIV1 based on recombinant engineering of infectious PIV clones. A variety of alterations can be produced in an isolated polynucleotide sequence encoding a targeted gene or genome segment, including a donor or recipient gene or genome segment in a recombinant HPIV1 genome or antigenome for incorporation into infectious clones. More specifically, to achieve desired structural and phenotypic changes in recombinant HPIV1, the invention allows for introduction of modifications which delete, substitute, introduce, or rearrange a selected nucleotide or nucleotide sequence from a parent genome or antigenome, as well as mutations which delete, substitute, introduce or rearrange whole gene(s) or genome segment(s), within a recombinant HPIV1.

Thus provided are modifications in recombinant HPIV1 of the invention which simply alter or ablate expression of a selected gene, e.g., by introducing a termination codon within a selected PIV coding sequence or altering its translational start site or RNA editing site, changing the position of a PIV gene relative to an operably linked promoter, introducing an upstream start codon to alter rates of expression, modifying (e.g., by changing position, altering an existing sequence, or substituting an existing sequence with a heterologous sequence) GS and/or GE transcription signals to alter phenotype (e.g., growth, temperature restrictions on transcription, etc.), and various other deletions, substitutions, additions and rearrangements that specify quantitative or qualitative changes in viral replication, transcription of selected gene(s), or translation of selected protein(s). In this context, any PIV gene or genome segment which is not essential for growth can be ablated or otherwise modified in a recombinant PIV to yield desired effects on virulence, pathogenesis, immunogenicity and other phenotypic characters. As for coding sequences, noncoding, leader, trailer and intergenic regions can be similarly deleted, substituted or modified and their phenotypic effects readily analyzed, e.g., by the use of minireplicons and recombinant PIV.

In addition, a variety of other genetic alterations can be produced in a recombinant HPIV1 genome or antigenome, alone or together with one or more attenuating mutations adopted from a biologically derived mutant PIV or other virus, e.g., to adjust growth, attenuation, immunogenicity, genetic stability or provide other advantageous structural and/or phenotypic effects. These additional types of mutations have been disclosed in the foregoing incorporated references and elsewhere and can be readily engineered into recombinant HPIV1 of the invention. For example, restriction site markers are routinely introduced within chimeric PIVs to facilitate cDNA construction and manipulation.

In addition to these changes, the order of genes in a recombinant HPIV1 construct can be changed, a PIV genome promoter replaced with its antigenome counterpart, portions of genes removed or substituted, and even entire genes deleted. Different or additional modifications in the sequence can be made to facilitate manipulations, such as the insertion of unique restriction sites in various intergenic regions or elsewhere. Nontranslated gene sequences can be removed to increase capacity for inserting foreign sequences.

Other mutations for incorporation into recombinant HPIV1 constructs of the invention include mutations directed toward cis-acting signals, which can be readily identified, e.g., by mutational analysis of PIV minigenomes. For example, insertional and deletional analysis of the leader and trailer and flanking sequences identifies viral promoters and transcription signals and provides a series of mutations associated with varying degrees of reduction of RNA replication or transcription. Saturation mutagenesis (whereby each position in turn is modified to each of the nucleotide alternatives) of these cis-acting signals also has identified many mutations that affect RNA replication or transcription. Any of these mutations can be inserted into a chimeric PIV antigenome or genome as described herein. Evaluation and manipulation of trans-acting proteins and cis-acting RNA sequences using the complete antigenome cDNA is assisted by the use of PIV minigenomes as described previously.

Additional mutations within recombinant HPIV1 viruses of the invention may also include replacement of the 3′ end of genome with its counterpart from antigenome, which is associated with changes in RNA replication and transcription. In one exemplary embodiment, the level of expression of specific PIV proteins, such as the protective HN and/or F antigens, can be increased by substituting the natural sequences with ones which have been made synthetically and designed to be consistent with efficient translation. In this context, it has been shown that codon usage can be a major factor in the level of translation of mammalian viral proteins (Haas et al., Current Biol. 6:315-324, 1996, incorporated herein by reference). Optimization by recombinant methods of the codon usage of the mRNAs encoding the HN and F proteins of recombinant HPIV1 provides improved expression for these genes.

In another exemplary embodiment, a sequence surrounding a translational start site (preferably including a nucleotide in the −3 position) of a selected HPIV1 gene is modified, alone or in combination with introduction of an upstream start codon, to modulate gene expression by specifying up- or down-regulation of translation. Alternatively, or in combination with other recombinant modifications disclosed herein, gene expression of a recombinant HPIV1 can be modulated by altering a transcriptional GS or GE signal of any selected gene(s) of the virus. In alternative embodiments, levels of gene expression in a recombinant HPIV1 candidate are modified at the level of transcription. In one aspect, the position of a selected gene in the PIV gene map can be changed to a more promoter-proximal or promoter-distal position, whereby the gene will be expressed more or less efficiently, respectively. According to this aspect, modulation of expression for specific genes can be achieved yielding reductions or increases of gene expression from two-fold, more typically four-fold, up to ten-fold or more compared to wild-type levels often attended by a commensurate decrease in expression levels for reciprocally, positionally substituted genes. These and other transpositioning changes yield novel recombinant HPIV1 viruses having attenuated phenotypes, for example due to decreased expression of selected viral proteins involved in RNA replication, or having other desirable properties such as increased antigen expression.

In other embodiments, recombinant HPIV1 viruses useful in immunogenic compositions can be conveniently modified to accommodate antigenic drift in circulating virus. Typically the modification will be in the HN and/or F proteins. An entire HN or F gene, or a genome segment encoding a particular immunogenic region thereof, from one PIV (HPIV1 or another HPIV) strain or group is incorporated into a recombinant HPIV1 genome or antigenome cDNA by replacement of a corresponding region in a recipient clone of a different PIV strain or group, or by adding one or more copies of the gene, such that multiple antigenic forms are represented. Progeny virus produced from the modified recombinant HPIV1 can then be used in immunization protocols against emerging PIV strains.

In preferred chimeric HPIV1 candidates of the invention, attenuation marked by replication in the lower and/or upper respiratory tract in an accepted animal model that is reasonably correlated with PIV replication and immunogenic activity in humans, e.g., hamsters, rhesus monkeys or chimpanzees, may be reduced by at least about two-fold, more often about 5-fold, 10-fold, or 20-fold, and preferably 50-100-fold and up to 1,000-fold or greater overall (e.g., as measured between 3-8 days following infection) compared to growth of the corresponding wild-type or mutant parental PIV strain.

Within the methods of the invention, additional genes or genome segments may be inserted into or proximate to a recombinant or chimeric HPIV1 genome or antigenome. For example, various supernumerary heterologous gene(s) or genome segment(s) can be inserted at any of a variety of sites within the recombinant genome or antigenome, for example at a position 3′ to N, between the N/P, P/M, and/or HN/L genes, or at another intergenic junction or non-coding region of the HPIV1 vector genome or antigenome. The inserted genes may be under common control with recipient genes, or may be under the control of an independent set of transcription signals. Genes of interest in this context include genes encoding cytokines, for example, an interleukin (e.g., interleukin 2 (IL-2), interleukin 4 (IL-4), interleukin 5 (IL-5), interleukin 6 (IL-6), interleukin 12 (IL-12), interleukin 18 (IL-18)), tumor necrosis factor alpha (TNFα), interferon gamma (IFNγ), or granulocyte-macrophage colony stimulating factor (GM-CSF), (see, e.g., U.S. application Ser. No. 09/614,285, filed Jul. 12, 2000, corresponding to U.S. Provisional Application Ser. No. 60/143,425 filed Jul. 13, 1999, incorporated herein by reference). Coexpression of these additional proteins provides the ability to modify and improve immune responses against recombinant HPIV1 of the invention both quantitatively and qualitatively.

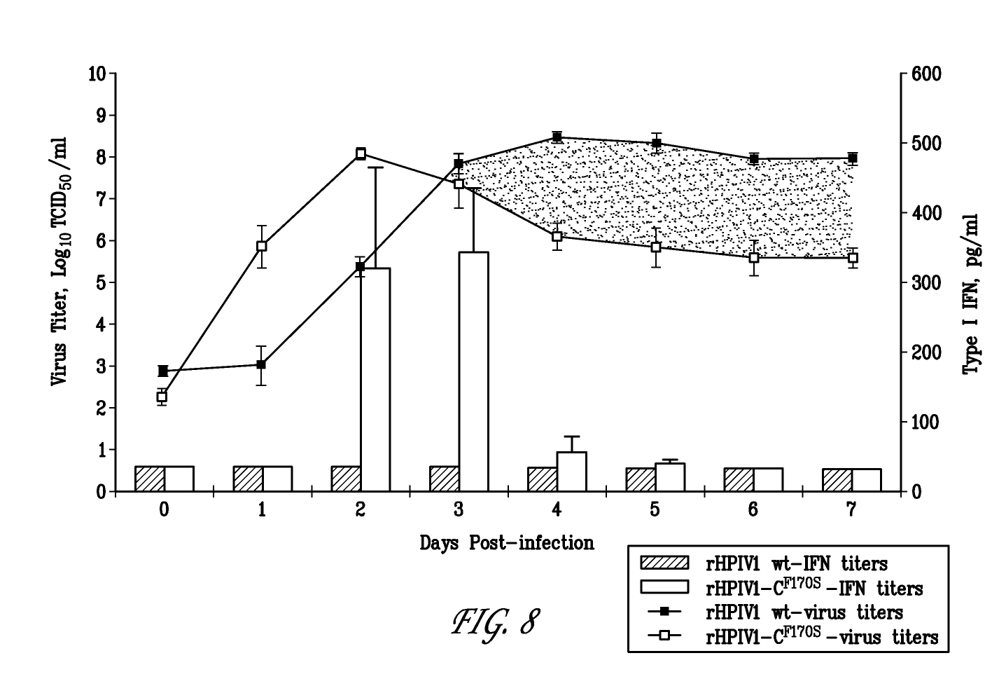

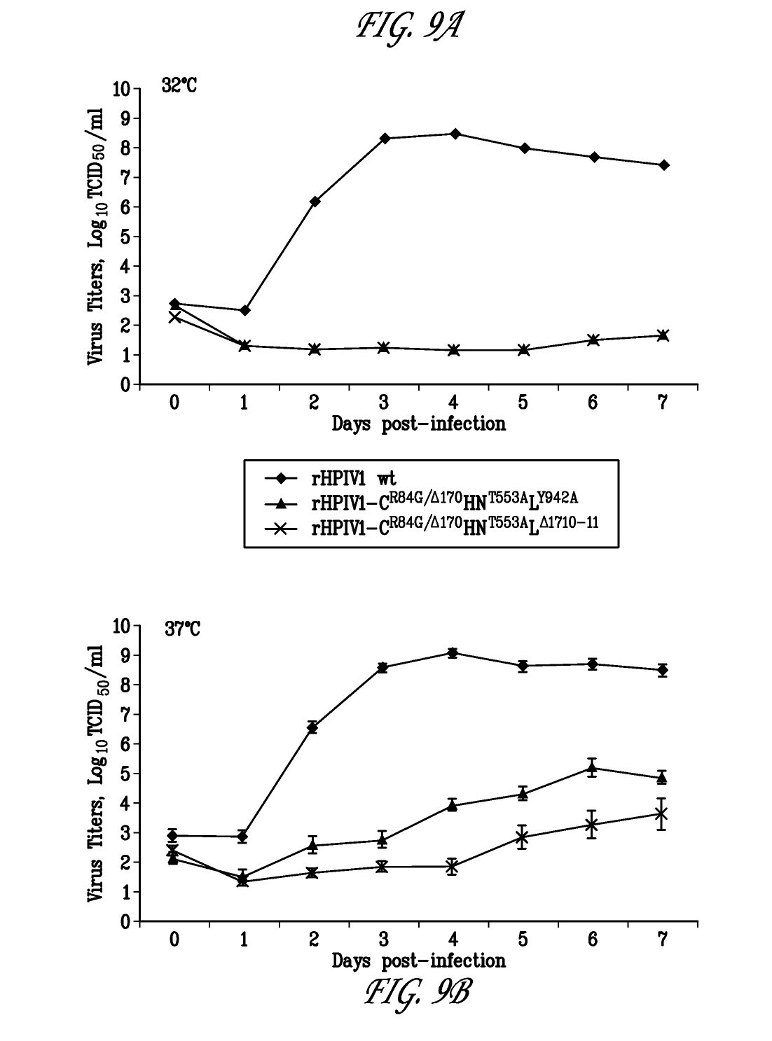

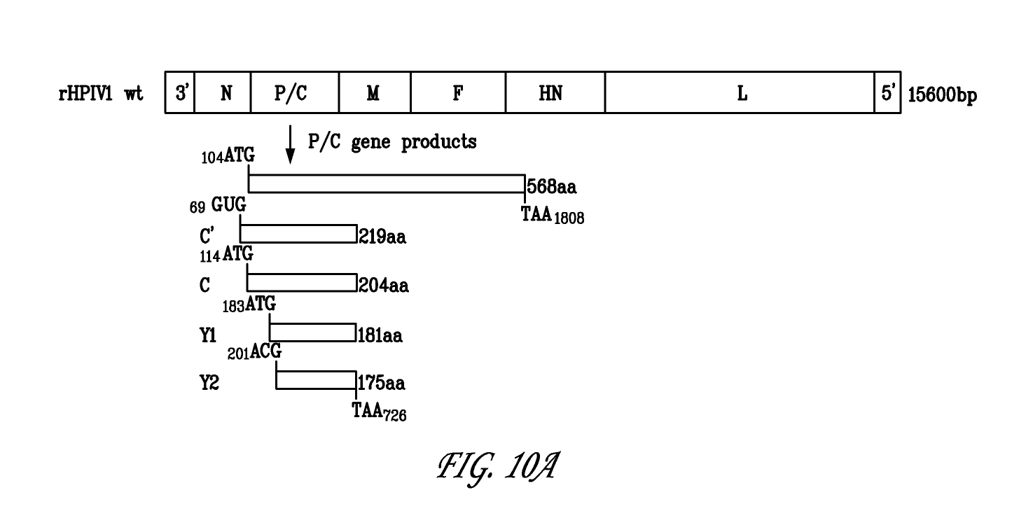

In yet additional embodiments of the invention, chimeric HPIV1 viruses are constructed using a HPIV1 “vector” genome or antigenome that is recombinantly modified to incorporate one or more antigenic determinants of a heterologous pathogen. The vector genome or antigenome is comprised of a partial or complete HPIV1 genome or antigenome, which may itself incorporate nucleotide modifications such as attenuating mutations. The vector genome or antigenome is modified to form a chimeric structure through incorporation of a heterologous gene or genome segment. More specifically, chimeric HPIV1 viruses of the invention are constructed through a cDNA-based virus recovery system that yields recombinant viruses that incorporate a partial or complete vector or “background” HPIV1 genome or antigenome combined with one or more “donor” nucleotide sequences encoding the heterologous antigenic determinant(s). In exemplary embodiments a HPIV1 vector genome or antigenome is modified to incorporate one or more genes or genome segments that encode antigenic determinant(s) of one or more heterologous PIVs (e.g., HPIV2 and/or HPIV3), and/or a non-PIV pathogen (e.g., RSV, human metapneumovirus, or measles virus). Thus constructed, chimeric HPIV1 viruses of the invention may elicit an immune response against a specific PIV, e.g., HPIV1, HPIV2, and/or HPIV3, or against a non-PIV pathogen. Alternatively, compositions and methods are provided employing a HPIV1-based chimeric virus to elicit a polyspecific immune response against multiple PIVs, e.g., HPIV1 and HPIV3, or against one or more HPIVs and a non-PIV pathogen such as measles virus. Exemplary construction of a chimeric, vector HPIV1 candidate virus is illustrated in FIG. 8 of WO 2003/043587. In preferred aspects of the invention, chimeric HPIV1 incorporate a partial or complete human HPIV1 incorporating one or more heterologous polynucleotide(s) encoding one or more antigenic determinants of the heterologous pathogen, which polynucleotides may be added to or substituted within the HPIV1 vector genome or antigenome to yield the chimeric HPIV1 recombinant. The chimeric HPIV1 virus thus acquires the ability to elicit an immune response in a selected host against the heterologous pathogen. In addition, the chimeric virus may exhibit other novel phenotypic characteristics compared to one or both of the vector PIV and heterologous pathogens.

The partial or complete vector genome or antigenome generally acts as a backbone into which heterologous genes or genome segments of a different pathogen are incorporated. Often, the heterologous pathogen is a different PIV from which one or more gene(s) or genome segment(s) is/are combined with, or substituted within, the vector genome or antigenome. In addition to providing novel immunogenic characteristics, the addition or substitution of heterologous genes or genome segments within the vector HPIV1 strain may confer an increase or decrease in attenuation, growth changes, or other desired phenotypic changes as compared with the corresponding phenotype(s) of the unmodified vector and donor viruses. Heterologous genes and genome segments from other PIVs that may be selected as inserts or additions within chimeric PIV of the invention preferably include genes or genome segments encoding the PIV N, P, M, F, HN and/or L protein(s) or one or more antigenic determinant(s) thereof.

Heterologous genes or genome segments of one PIV may be added as a supernumerary genomic element to a partial or complete genome or antigenome of HPIV1. Alternatively, one or more heterologous gene(s) or genome segment(s) of one PIV may be substituted at a position corresponding to a wild-type gene order position of a counterpart gene(s) or genome segment(s) that is deleted within the HPIV1 vector genome or antigenome. In yet additional embodiments, the heterologous gene or genome segment is added or substituted at a position that is more promoter-proximal or promotor-distal compared to a wild-type gene order position of the counterpart gene or genome segment within the vector genome or antigenome to enhance or reduce, respectively, expression of the heterologous gene or genome segment.

The introduction of heterologous immunogenic proteins, protein domains and immunogenic epitopes to produce chimeric HPIV1 is particularly useful to generate novel immune responses in an immunized host. Addition or substitution of an immunogenic gene or genome segment from a “donor” pathogen within a recipient HPIV1 vector genome or antigenome can generate an immune response directed against the donor pathogen, the HPIV1 vector, or against both the donor pathogen and vector.

General methods and compositions useful within the invention for engineering chimeric PIV and PIV “vector” viruses are provided by Durbin et al., Virology 235:323-332, 1997; Skiadopoulos et al., J. Virol. 72:1762-1768, 1998; Tao et al., J Virol 72:2955-2961, 1998; Skiadopoulos et al., J. Virol. 73:1374-1381, 1999; Skiadopoulos et al., Vaccine 18:503-510, 1999; Tao et al., Vaccine 17:1100-1108, 1999; Tao et al., Vaccine 18:1359-1366, 2000; U.S. patent application Ser. No. 09/083,793, filed May 22, 1998; U.S. patent application Ser. No. 09/458,813, filed Dec. 10, 1999; U.S. patent application Ser. No. 09/459,062, filed Dec. 10, 1999; U.S. Provisional Application No. 60/047,575, filed May 23, 1997 (corresponding to International Publication No. WO 98/53078); and U.S. Provisional Application No. 60/059,385, filed Sep. 19, 1997; U.S. Provisional Application No. 60/170,195; and PCT publication WO 01/42445A2 published Jun. 14, 2001, each incorporated herein by reference.

Chimeric HPIV1 of the invention may also be constructed that express a chimeric protein, for example an immunogenic glycoprotein having a cytoplasmic tail and/or transmembrane domain specific to a HPIV1 vector fused to a heterologous ectodomain of a different PIV or non-PIV pathogen to provide a fusion protein that elicits an immune response against the heterologous pathogen. For example, a heterologous genome segment encoding a glycoprotein ectodomain from a HPIV2 or HPIV3 HN or F glycoprotein may be joined with a genome segment encoding the corresponding HPIV1 HN or F glycoprotein cytoplasmic and transmembrane domains to form a HPIV1-2 or HPIV1-3 chimeric glycoprotein that elicits an immune response against HPIV1 and HPIV2 or HPIV3.

Briefly, HPIV1 of the invention expressing a chimeric glycoprotein comprise a major nucleocapsid (N) protein, a nucleocapsid phosphoprotein (P), a large polymerase protein (L), and a HPIV1 vector genome or antigenome that is modified to encode a chimeric glycoprotein. The chimeric glycoprotein incorporates one or more heterologous antigenic domains, fragments, or epitopes of a second, antigenically distinct HPIV. Preferably, this is achieved by substitution within the HPIV1 vector genome or antigenome of one or more heterologous genome segments of the second HPIV that encode one or more antigenic domains, fragments, or epitopes, whereby the genome or antigenome encodes the chimeric glycoprotein that is antigenically distinct from the parent, vector virus.

In more detailed aspects, the heterologous genome segment or segments preferably encode a glycoprotein ectodomain or immunogenic portion or epitope thereof, and optionally include other portions of the heterologous or “donor” glycoprotein, for example both an ectodomain and transmembrane region that are substituted for counterpart glycoprotein ecto- and transmembrane domains in the vector genome or antigenome. Preferred chimeric glycoproteins in this context may be selected from HPIV HN and/or F glycoproteins, and the vector genome or antigenome may be modified to encode multiple chimeric glycoproteins. In preferred embodiments, the HPIV1 vector genome or antigenome is a partial genome or antigenome and the second, antigenically distinct HPIV is either HPIV2 or HPIV3. In one exemplary embodiment, both glycoprotein ectodomain(s) of HPIV2 or HPIV3 HN and F glycoproteins are substituted for corresponding HN and F glycoprotein ectodomains in the HPIV1 vector genome or antigenome. In another exemplary embodiment, HPIV2 or HPIV3 ectodomain and transmembrane regions of one or both HN and/or F glycoproteins are fused to one or more corresponding PIV1 cytoplasmic tail region(s) to form the chimeric glycoprotein. Further details concerning these aspects of the invention are provided in United States patent application entitled “CONSTRUCTION AND USE OF RECOMBINANT PARAINFLUENZA VIRUSES EXPRESSING A CHIMERIC GLYCOPROTEIN”, filed on Dec. 10, 1999 by Tao et al. and identified by Attorney Docket No. 17634-000340, incorporated herein by reference.

To construct chimeric HPIV1 viruses of the invention carrying a heterologous antigenic determinant of a non-PIV pathogen, a heterologous gene or genome segment of the donor pathogen may be added or substituted at any operable position in the vector genome or antigenome. In one embodiment, heterologous genes or genome segments from a non-PIV pathogen can be added (i.e., without substitution) within a HPIV1 vector genome or antigenome to create novel immunogenic properties within the resultant clone (see, e.g., FIG. 8). In these cases, the heterologous gene or genome segment may be added as a supernumerary gene or genome segment, optionally for the additional purpose of attenuating the resultant chimeric virus, in combination with a complete HPIV1 vector genome or antigenome. Alternatively, the heterologous gene or genome segment may be added in conjunction with deletion of a selected gene or genome segment in the vector genome or antigenome.

In some embodiments of the invention, the heterologous gene or genome segment is added at an intergenic position within the partial or complete HPIV1 vector genome or antigenome. Alternatively, the gene or genome segment can be inserted within other noncoding regions of the genome, for example, within 5′ or 3′ noncoding regions or in other positions where noncoding nucleotides occur within the vector genome or antigenome. In one aspect, the heterologous gene or genome segment is inserted at a non-coding site overlapping a cis-acting regulatory sequence within the vector genome or antigenome, e.g., within a sequence required for efficient replication, transcription, and/or translation. These regions of the vector genome or antigenome represent target sites for disruption or modification of regulatory functions associated with introduction of the heterologous gene or genome segment.

As used herein, the term “gene” generally refers to a portion of a subject genome, e.g., a HPIV1 genome, encoding an mRNA and typically begins at the upstream end with a gene-start (GS) signal and ends at the downstream end with the gene-end (GE) signal. The term gene is also interchangeable with the term “translational open reading frame”, or ORF, particularly in the case where a protein, such as the C protein, is expressed from an additional ORF rather than from a unique mRNA. The viral genome of all PIVs also contains extragenic leader and trailer regions, possessing part of the promoters required for viral replication and transcription, as well as non-coding and intergenic regions. Transcription initiates at the 3′ end and proceeds by a sequential stop-start mechanism that is guided by short conserved motifs found at the gene boundaries. The upstream end of each gene contains a gene-start (GS) signal, which directs initiation of its respective mRNA. The downstream terminus of each gene contains a gene-end (GE) motif which directs polyadenylation and termination.

To construct chimeric HPIV1 viruses of the invention, one or more PIV gene(s) or genome segment(s) may be deleted, inserted or substituted in whole or in part. This means that partial or complete deletions, insertions and substitutions may include open reading frames and/or cis-acting regulatory sequences of any one or more of the PIV genes or genome segments. By “genome segment” is meant any length of continuous nucleotides from the PIV genome, which might be part of an ORF, a gene, or an extragenic region, or a combination thereof. When a subject genome segment encodes an antigenic determinant, the genome segment encodes at least one immunogenic epitope capable of eliciting a humoral or cell mediated immune response in a mammalian host. The genome segment may also encode an immunogenic fragment or protein domain. In other aspects, the donor genome segment may encode multiple immunogenic domains or epitopes, including recombinantly synthesized sequences that comprise multiple, repeating or different, immunogenic domains or epitopes.

In some embodiments of the invention, the chimeric HPIV1 bears one or more major antigenic determinants of a human PIV, or multiple human PIVs, including HPIV1, HPIV2 or HPIV3. These preferred candidates elicit an effective immune response in humans against one or more selected HPIVs. As noted above, the antigenic determinant(s) that elicit(s) an immune response against HPIV may be encoded by the HPIV1 vector genome or antigenome, or may be inserted within or joined to the PIV vector genome or antigenome as a heterologous gene or gene segment. The major protective antigens of human PIVs are their HN and F glycoproteins. However, all PIV genes are candidates for encoding antigenic determinants of interest, including internal protein genes which may encode such determinants as, for example, CTL epitopes.

Chimeric HPIV1 viruses of the invention might bear one or more major antigenic determinants from each of a plurality of HPIVs or from a HPIV and a non-PIV pathogen. Chimeric HPIV1 viruses thus constructed include one or more heterologous gene(s) or genome segment(s) encoding antigenic determinant(s) of the same or a heterologous (for example HPIV2 or HPIV3) PIV. These and other constructs yield chimeric PIVs that elicit either a mono- or poly-specific immune response in humans to one or more HPIVs. Such aspects of the invention are provided in U.S. patent application Ser. No. 09/083,793, filed May 22, 1998; U.S. patent application Ser. No. 09/458,813, filed Dec. 10, 1999; U.S. patent application Ser. No. 09/459,062, filed Dec. 10, 1999; U.S. Provisional Application No. 60/047,575, filed May 23, 1997 (corresponding to International Publication No. WO 98/53078), U.S. Provisional Application No. 60/059,385, filed Sep. 19, 1997; U.S. Provisional Application No. 60/170,195 filed Dec. 10, 1999; and U.S. patent application Ser. No. 09/733,692, filed Dec. 8, 2000 (corresponding to International Publication No. WO 01/42445A2), each incorporated herein by reference.

In other exemplary aspects of the invention, chimeric HPIV1 incorporate a HPIV1 vector genome or antigenome modified to express one or more major antigenic determinants of non-PIV pathogen, for example measles virus. The methods of the invention are generally adaptable for incorporation of antigenic determinants from a wide range of additional pathogens within chimeric HPIV1 candidates. In this regard the invention also provides for development of candidates for eliciting immune responses against subgroup A and subgroup B respiratory syncytial viruses (RSV), mumps virus, human papilloma viruses, type 1 and type 2 human immunodeficiency viruses, herpes simplex viruses, cytomegalovirus, rabies virus, Epstein Barr virus, filoviruses, bunyaviruses, flaviviruses, alphaviruses and influenza viruses, among other pathogens. Pathogens that may be targeted according to the methods of the invention include viral and bacterial pathogens, as well as protozoans and multicellular pathogens. Useful antigenic determinants from many important human pathogens in this context are known or readily identified for incorporation within chimeric HPIV1 of the invention. Thus, major antigens have been identified for the foregoing exemplary pathogens, including the measles virus HA and F proteins; the F, G, SH and M2 proteins of RSV, mumps virus HN and F proteins, human papilloma virus L1 protein, type 1 or type 2 human immunodeficiency virus gp160 protein, herpes simplex virus and cytomegalovirus gB, gC, gD, gE, gG, gH, gI, gJ, gK, gL, and gM proteins, rabies virus G protein, Epstein Barr Virus gp350 protein; filovirus G protein, bunyavirus G protein, flavivirus E and NS1 proteins, metapneumovirus G and F proteins, and alphavirus E protein. These major antigens, as well as other antigens known in the art for the enumerated pathogens and others, are well characterized to the extent that many of their antigenic determinants, including the full length proteins and their constituent antigenic domains, fragments and epitopes, are identified, mapped and characterized for their respective immunogenic activities.

Among the numerous, exemplary mapping studies that identify and characterize major antigens of diverse pathogens for use within the invention are epitope mapping studies directed to the hemagglutinin-neuraminidase (HN) gene of HPIV (van Wyke Coelingh et al., J. Virol. 61:1473-1477, 1987, incorporated herein by reference). This report provides detailed antigenic structural analyses for 16 antigenic variants of HPIV3 variants selected by using monoclonal antibodies (MAbs) to the HN protein which inhibit neuraminidase, hemagglutination, or both activities. Each variant possessed a single-point mutation in the HN gene, coding for a single amino acid substitution in the HN protein. Operational and topographic maps of the HN protein correlated well with the relative positions of the substitutions. Computer-assisted analysis of the HN protein predicted a secondary structure composed primarily of hydrophobic β sheets interconnected by random hydrophilic coil structures. The HN epitopes were located in predicted coil regions. Epitopes recognized by MAbs which inhibit neuraminidase activity of the virus were located in a region which appears to be structurally conserved among several paramyxovirus HN proteins and which may represent the sialic acid-binding site of the HN molecule.

This exemplary work, employing conventional antigenic mapping methods, identified single amino acids which are important for the integrity of HN epitopes. Most of these epitopes are located in the C-terminal half of the molecule, as expected for a protein anchored at its N terminus (Elango et al., J. Virol. 57:481-489, 1986). Previously published operational and topographic maps of the PIV3 HN indicated that the MAbs employed recognized six distinct groups of epitopes (I to VI) organized into two topographically separate sites (A and B), which are partially bridged by a third site (C). These groups of epitopes represent useful candidates for antigenic determinants that may be incorporated, alone or in various combinations, within chimeric HPIV1 viruses of the invention. (See, also, Coelingh et al., Virology 143:569-582, 1985; Coelingh et al., Virology 162:137-143, 1988; Ray et al., Virology 148:232-236, 1986; Rydbeck et al., J. Gen. Virol. 67:1531-1542, 1986, each incorporated herein by reference).

Additional studies by van Wyke Coelingh et al. (J. Virol. 63:375-382, 1989) provide further information relating to selection of PIV antigenic determinants for use within the invention. In this study, twenty-six monoclonal antibodies (MAbs) (14 neutralizing and 12 nonneutralizing) were used to examine the antigenic structure, biological properties, and natural variation of the fusion (F) glycoprotein of HPIV3. Analysis of laboratory-selected antigenic variants and of PIV3 clinical isolates indicated that the panel of MAbs recognizes at least 20 epitopes, 14 of which participate in neutralization. Competitive binding assays confirmed that the 14 neutralization epitopes are organized into three nonoverlapping principal antigenic regions (A, B, and C) and one bridge site (AB), and that the 6 nonneutralization epitopes form four sites (D, E, F, and G). Most of the neutralizing MAbs were involved in nonreciprocal competitive binding reactions, suggesting that they induce conformational changes in other neutralization epitopes.

Other antigenic determinants for use within the invention have been identified and characterized for respiratory syncytial virus (RSV). For example, Beeler et al., J. Virol. 63:2941-2950, 1989, incorporated herein by reference, employed eighteen neutralizing monoclonal antibodies (MAbs) specific for the fusion glycoprotein of the A2 strain of RSV to construct a detailed topological and operational map of epitopes involved in RSV neutralization and fusion. Competitive binding assays identified three nonoverlapping antigenic regions (A, B, and C) and one bridge site (AB). Thirteen MAb-resistant mutants (MARMs) were selected, and the neutralization patterns of the MAbs with either MARMs or RSV clinical strains identified a minimum of 16 epitopes. MARMs selected with antibodies to six of the site A and AB epitopes displayed a small-plaque phenotype, which is consistent with an alteration in a biologically active region of the F molecule. Analysis of MARMs also indicated that these neutralization epitopes occupy topographically distinct but conformationally interdependent regions with unique biological and immunological properties. Antigenic variation in F epitopes was then examined by using 23 clinical isolates (18 subgroup A and 5 subgroup B) in cross-neutralization assays with the 18 anti-F MAbs. This analysis identified constant, variable, and hypervariable regions on the molecule and indicated that antigenic variation in the neutralization epitopes of the RSV F glycoprotein is the result of a noncumulative genetic heterogeneity. Of the 16 epitopes, 8 were conserved on all or all but 1 of 23 subgroup A or subgroup B clinical isolates. These antigenic determinants, including the full length proteins and their constituent antigenic domains, fragments and epitopes, all represent useful candidates for integration within chimeric PIV of the invention to elicit novel immune responses as described above. (See also, Anderson et al., J. Infect. Dis. 151:626-633, 1985; Coelingh et al., J. Virol. 63:375-382, 1989; Fenner et al., Scand. J. Immunol. 24:335-340, 1986; Fernie et al., Proc. Soc. Exp. Biol. Med. 171:266-271, 1982; Sato et al., J. Gen. Virol. 66:1397-1409, 1985; Walsh et al., J. Gen. Virol. 67:505-513, 1986, and Olmsted et al., J. Virol. 63:411-420, 1989, each incorporated herein by reference).

To express antigenic determinants of heterologous PIVs and non-PIV pathogens, the invention provides numerous methods and constructs. In certain embodiments, a transcription unit comprising an open reading frame (ORF) of a gene encoding an antigenic protein (e.g., the measles virus HA gene) is added to a HPIV1 vector genome or antigenome at various positions, yielding exemplary chimeric PIV1/measles candidates. In exemplary embodiments, chimeric HPIV1 viruses are engineered that incorporate heterologous nucleotide sequences encoding protective antigens from respiratory syncytial virus (RSV) to produce infectious, attenuated viruses. The cloning of RSV cDNA and other disclosure is provided in U.S. Provisional Patent Application No. 60/007,083, filed Sep. 27, 1995; U.S. patent application Ser. No. 08/720,132, filed Sep. 27, 1996; U.S. Provisional Patent Application No. 60/021,773, filed Jul. 15, 1996; U.S. Provisional Patent Application No. 60/046,141, filed May 9, 1997; U.S. Provisional Patent Application No. 60/047,634, filed May 23, 1997; U.S. patent application Ser. No. 08/892,403, filed Jul. 15, 1997 (corresponding to International Publication No. WO 98/02530); U.S. patent application Ser. No. 09/291,894, filed on Apr. 13, 1999; International Application No. PCT/US00/09696, filed Apr. 12, 2000, corresponding to U.S. Provisional Patent Application Ser. No. 60/129,006, filed on Apr. 13, 1999; Collins et al., Proc Nat. Acad. Sci. U.S.A. 92:11563-11567, 1995; Bukreyev et al., J. Virol. 70:6634-41, 1996, Juhasz et al., J. Virol. 71:5814-5819, 1997; Durbin et al., Virology 235:323-332, 1997; He et al. Virology 237:249-260, 1997; Baron et al. J. Virol. 71:1265-1271, 1997; Whitehead et al., Virology 247:232-9, 1998a; Whitehead et al., J. Virol. 72:4467-4471, 1998b; Jin et al. Virology 251:206-214, 1998; and Whitehead et al., J. Virol. 73:3438-3442, 1999, and Bukreyev et al., Proc. Nat. Acad. Sci. U.S.A. 96:2367-72, 1999, each incorporated herein by reference in its entirety for all purposes). Other reports and discussion incorporated or set forth herein identify and characterize RSV antigenic determinants that are useful within the invention.

PIV chimeras incorporating one or more RSV antigenic determinants, preferably comprise a HPIV1 vector genome or antigenome combined with a heterologous gene or genome segment encoding an antigenic RSV glycoprotein, protein domain (e.g., a glycoprotein ectodomain) or one or more immunogenic epitopes. In one embodiment, one or more genes or genome segments from RSV F and/or G genes is/are combined with the vector genome or antigenome to form the chimeric HPIV1. Certain of these constructs will express chimeric proteins, for example fusion proteins having a cytoplasmic tail and/or transmembrane domain of HPIV1 fused to an ectodomain of RSV to yield a novel attenuated virus that optionally elicits a multivalent immune response against both PIV1 and RSV.

Any of the embodiments described herein may be practiced utilizing either the viruses designated as rHPIV1-C^{R84G/Δ170}HN^{T553A}L^{Y942A} and rHPIV1-C^{R84G/Δ170}HN^{T553A}L^{Δ1710-11}, or the viruses having the HPIV1 P(C-) structure.

Considering the epidemiology of RSV and HPIV1, HPIV2, and HPIV3, it will be optimal to administer immunogenic compositions of the invention in a predetermined, sequential schedule. RSV and HPIV3 cause significant illness within the first four months of life whereas most of the illness caused by HPIV1 and HPIV2 occur after six months of age (Chanock et al., in Parainfluenza Viruses, Knipe et al. (Eds.), pp. 1341-1379, Lippincott Williams & Wilkins, Philadelphia, 2001; Collins et al., In Fields Virology, Vol. 1, pp. 1205-1243, Lippincott-Raven Publishers, Philadelphia, 1996; Reed et al., J. Infect. Dis. 175:807-13, 1997, each incorporated herein by reference). Accordingly, certain sequential immunization protocols of the invention will involve administration of immunogenic compositions to elicit a response against HPIV3 and/or RSV (e.g., as a combined formulation) two or more times early in life, with the first dose administered at or before one month of age, followed by an immunogenic composition directed against HPIV1 and/or HPIV2 at about four and six months of age.

The invention therefore provides novel combinatorial immunogenic compositions and coordinate immunization protocols for multiple pathogenic agents, including multiple PIV's and/or PIV and a non-PIV pathogen. These methods and formulations effectively target early immunization against RSV and PIV3. One preferred immunization sequence employs one or more live attenuated viruses that elicit a response against RSV and PIV3 as early as one month of age (e.g., at one and two months of age) followed by a bivalent PIV1 and PIV2 immunogenic composition at four and six months of age. It is thus desirable to employ the methods of the invention to administer multiple PIV immunogenic compositions, including one or more chimeric PIV compositions, coordinately, e.g., simultaneously in a mixture or separately in a defined temporal sequence (e.g., in a daily or weekly sequence), wherein each virus preferably expresses a different heterologous protective antigen. Such a coordinate/sequential immunization strategy, which is able to induce secondary antibody responses to multiple viral respiratory pathogens, provides a highly powerful and extremely flexible immunization regimen that is driven by the need to immunize against each of the three PIV viruses and other pathogens in early infancy.

Other sequential immunizations according to the invention permit the induction of the high titer of antibody targeted to a heterologous pathogen, such as measles. In one embodiment, young infants (e.g. 2-4 month old infants) are immunized with an attenuated HPIV3 or a chimeric HPIV1 and/or HPIV3 virus that elicits an immune response against HPIV3 and/or measles (for example a chimeric HPIV1 or HPIV3 virus expressing the measles virus HA protein and also adapted to elicit an immune response against HPIV3). Subsequently, e.g., at four months of age the infant is again immunized but with a different, secondary vector construct, such as a rHPIV1 virus expressing the measles virus HA gene and the HPIV1 antigenic determinants as functional, obligate glycoproteins of the vector. Following the first immunization, the subject will demonstrate a primary antibody response to both the PIV3 HN and F proteins and to the measles virus HA protein, but not to the PIV1 HN and F protein. Upon secondary immunization with the rHPIV1 expressing the measles virus HA, the subject will be readily infected with the immunizing virus because of the absence of antibody to the PIV1 HN and F proteins and will develop both a primary antibody response to the PIV1 HN and F protective antigens and a high titered secondary antibody response to the heterologous measles virus HA protein. This sequential immunization strategy, preferably employing different serotypes of PIV as primary and secondary vectors, effectively circumvents immunity that is induced to the primary vector, a factor ultimately limiting the usefulness of vectors with only one serotype. The success of sequential immunization with rHPIV3 and rHPIV3-1 virus candidates as described above has been reported (Tao et al., Vaccine 17:1100-8, 1999, incorporated herein by reference), but with the limitation of decreased immunogenicity of rHPIV3-1 against HPIV1 challenge. The present invention, in which the backbone of the booster virus is antigenically unrelated to the primary virus or vector, overcomes this important limitation.

Further in accordance with these aspects of the invention, exemplary coordinate immunization protocols may incorporate two, three, four and up to six or more separate HPIV viruses administered simultaneously (e.g., in a polyspecific mixture) in a primary immunization step, e.g., at one, two or four months of age. For example, two or more HPIV1-based viruses for use in immunogenic compositions can be administered that separately express one or more antigenic determinants (i.e., whole antigens, immunogenic domains, or epitopes) selected from the G protein of RSV subgroup A, the F protein of RSV subgroup A, the G protein of RSV subgroup B, the F protein of RSV subgroup B, the HA protein of measles virus, and/or the F protein of measles virus. Coordinate booster administration of these same PIV1-based constructs can be repeated at two months of age. Subsequently, e.g., at four months of age, a separate panel of 2-6 or more antigenically distinct (referring to vector antigenic specificity) live attenuated HPIV1-based recombinant viruses can be administered in a secondary immunization step. For example, secondary immunization may involve concurrent administration of a mixture or multiple formulations that contain(s) multiple HPIV1 constructs that collectively express RSV G from subgroup A, RSV F from subgroup A, RSV F from subgroup B, RSV G from subgroup B, measles virus HA, and/or measles virus F, or antigenic determinants from any combination of these proteins. This secondary immunization provides a boost in immunity to each of the heterologous RSV and measles virus proteins or antigenic determinant(s) thereof. At six months of age, a tertiary immunization step involving administration of one to six or more separate live attenuated HPIV1-2 or HPIV1-3 vector-based recombinants can be coordinately administered that separately or collectively express RSV G from subgroup A, RSV F from subgroup A, RSV G from subgroup B, RSV F from subgroup B, measles virus HA, and/or measles virus F, or antigenic determinant(s) thereof. Optionally at this step in the immunization protocol, rHPIV3 and rHPIV1 may be administered in booster formulations. In this way, the strong immunity characteristic of secondary antibody to HPIV1, HPIV2, HPIV3, RSV A, RSV B, and measles viruses are all induced within the first six months of infancy. Such a coordinate/sequential immunization strategy, which is able to induce secondary antibody responses to multiple viral respiratory pathogens, provides a highly powerful and extremely flexible immunization regimen that is driven by the need to immunize against each of the three PIV viruses and other pathogens in early infancy.

The present invention thus overcomes the difficulties inherent in prior approaches to development of vector based immunogenic compositions and provides unique opportunities for immunization of infants during the first year of life against a variety of human pathogens. Previous studies in developing live-attenuated HPIV indicate that, unexpectedly, rPIVs and their attenuated and chimeric derivatives have properties which make them uniquely suited among the nonsegmented negative strand RNA viruses as vectors to express foreign proteins to provide immunogenic compositions against a variety of human pathogens. The skilled artisan would not have predicted that the human PIVs, which tend to grow substantially less well than the model nonsegmented negative strand viruses and which typically have been underrepresented with regard to molecular studies, would prove to have characteristics which are highly favorable as vectors. It is also surprising that the intranasal route of administration of these immunogenic compositions has proven a very efficient means to stimulate a robust local and systemic immune response against both the vector and the expressed heterologous antigen. Furthermore, this route provides additional advantages for immunization against heterologous pathogens which infect the respiratory tract or elsewhere.

The present invention provides major advantages over previous attempts to immunize young infants against measles virus and other microbial pathogens. First, the HPIV1 recombinant vector into which the protective antigen or antigens of heterologous pathogens such as measles virus are inserted can be attenuated in a finely adjusted manner by incorporation of one or more attenuating mutations or other modifications to attenuate the virus for the respiratory tract of the very young, seronegative or seropositive human infant. An extensive history of prior clinical evaluation and practice (see, e.g., Karron et al., Pediatr. Infect. Dis. J. 15:650-654, 1996; Karron et al., J. Infect. Dis. 171:1107-1114, 1995a; Karron et al., J. Infect. Dis. 172:1445-1450, 1995, each incorporated herein by reference) greatly facilitates evaluation of derivatives of these recombinants bearing foreign protective antigens in the very young human infant.

Yet another advantage of the invention is that chimeric HPIV1 bearing heterologous sequences will replicate efficiently in vitro to enable large scale production of virus for use in immunogenic compositions. This is in contrast to the replication of some single-stranded, negative-sense RNA viruses which can be inhibited in vitro by the insertion of a foreign gene (Bukreyev et al., J. Virol. 70:6634-41, 1996). Also, the presence of three antigenic serotypes of HPIV, each of which causes significant disease in humans and hence can serve simultaneously as vector and immunogen, presents a unique opportunity to sequentially immunize the infant with antigenically distinct variants of HPIV each bearing the same foreign protein. In this manner the sequential immunization permits the development of a primary immune response to the foreign protein which can be boosted during subsequent infections with the antigenically distinct HPIV also bearing the same or a different foreign protein or proteins, i.e., the protective antigen of measles virus or of another microbial pathogen. It is also likely that readministration of homologous HPIV vectors will also boost the response to both HPIV and the foreign antigen since the ability to cause multiple reinfections in humans is an unusual but characteristic attribute of the HPIVs (Collins et al., In “Fields Virology”, B. N. Fields, D. M. Knipe, P. M. Howley, R. M. Chanock, J. L. Melnick, T. P. Monath, B. Roizman, and S. E. Straus, Eds., Vol. 1, pp. 1205-1243. Lippincott-Raven Publishers, Philadelphia, 1996).

Yet another advantage of the invention is that the introduction of a gene unit into a HPIV1 vector has several highly desirable effects for the production of attenuated viruses. First, the insertion of gene units expressing, for example, the HA of measles virus or the HN of PIV2 can specify a host range phenotype on the HPIV1 vector, i.e., where the resulting HPIV1 vector replicates efficiently in vitro but is restricted in replication in vivo in both the upper and lower respiratory tracts. Thus, the insertion of a gene unit expressing a viral protective antigen as an attenuating factor for the HPIV1 vector is a desirable property in live attenuated viruses of the invention.

The HPIV1 vector system has unique advantages over other members of the single-stranded, negative-sense viruses of the Order Mononegavirales. First, most other mononegaviruses that have been used as vectors are not derived from human pathogens (e.g., murine HPIV1 (Sendai virus) (Sakai et al., FEBS Lett. 456:221-6, 1999), vesicular stomatitis virus (VSV) which is a bovine pathogen (Roberts et al., J. Virol. 72:4704-11, 1998), and canine PIV2 (SV5) (He et al., Virology 237:249-60, 1997)). For these nonhuman viruses, little or only weak immunity would be conferred against any human virus by antigens present in the vector backbone. Thus, a nonhuman virus vector expressing a supernumerary gene for a human pathogen would induce resistance only against that single human pathogen. In addition, use of viruses such as VSV, SV5, rabies, or Sendai virus as vector would expose subjects to viruses that they likely would not otherwise encounter during life. Infection with, and immune responses against, such nonhuman viruses would be of marginal benefit and would pose safety concerns, because there is little experience of infection with these viruses in humans.

An important and specific advantage of the HPIV1 vector system is that its preferred route of administration is the intranasal route, which mimics natural infection, will induce both mucosal and systemic immunity and reduces the neutralizing and immunosuppressive effects of maternally-derived serum IgG that is present in infants. While these same advantages theoretically are possible for using RSV as a vector, for example, we have found that RSV replication is strongly inhibited by inserts of greater than approximately 500 bp (Bukreyev et al., Proc. Natl. Acad. Sci. USA 96:2367-72, 1999). In contrast, as described herein, HPIV1 can readily accommodate several large gene inserts. The finding that recombinant RSV is unsuitable for bearing large inserts, whereas recombinant PIVs are highly suitable, represents unexpected results.

It might be proposed that some other viral vector could be given intranasally to obtain similar benefits as shown for PIV vectors, but this has not been successful to date. For example, the MVA strain of vaccinia virus expressing the protective antigens of HPIV3 was evaluated as a live attenuated intranasal vaccine against HPIV3. Although this vector appeared to be a very efficient expression system in cell culture, it was inexplicably inefficient in inducing resistance in the upper respiratory tract of primates (Durbin et al., Vaccine 16:1324-30, 1998) and was inexplicably inefficient in inducing a protective response in the presence of passive serum antibodies (Durbin et al., J. Infect. Dis. 179:1345-51, 1999). In contrast, PIV3 and RSV vaccine candidates have been found to be protective in the upper and lower respiratory tract of non-human primates, even in the presence of passive serum antibodies (Crowe et al., Vaccine 13:847-855, 1995; Durbin et al., J. Infect. Dis. 179:1345-51, 1999).

As noted above, the invention permits a wide range of alterations to be recombinantly produced within the HPIV1 genome or antigenome, yielding defined mutations that specify desired phenotypic changes. As also noted above, defined mutations can be introduced by a variety of conventional techniques (e.g., site-directed mutagenesis) into a cDNA copy of the genome or antigenome. The use of genomic or antigenomic cDNA subfragments to assemble a complete genome or antigenome cDNA as described herein has the advantage that each region can be manipulated separately, where small cDNA constructs provide for better ease of manipulation than large cDNA constructs, and then readily assembled into a complete cDNA. Thus, the complete antigenome or genome cDNA, or a selected subfragment thereof, can be used as a template for oligonucleotide-directed mutagenesis. This can be through the intermediate of a single-stranded phagemid form, such as using the MUTA-gen® kit of Bio-Rad Laboratories (Richmond, Calif.), or a method using the double-stranded plasmid directly as a template such as the Chameleon® mutagenesis kit of Stratagene (La Jolla, Calif.), or by the polymerase chain reaction employing either an oligonucleotide primer or a template which contains the mutation(s) of interest. A mutated subfragment can then be assembled into the complete antigenome or genome cDNA. A variety of other mutagenesis techniques are known and can be routinely adapted for use in producing the mutations of interest in a PIV antigenome or genome cDNA of the invention.

Thus, in one illustrative embodiment mutations are introduced by using the MUTA-gene® phagemid in vitro mutagenesis kit available from Bio-Rad Laboratories. In brief, cDNA encoding a PIV genome or antigenome is cloned into the plasmid pTZ18U, and used to transform CJ236 cells (Life Technologies). Phagemid preparations are prepared as recommended by the manufacturer. Oligonucleotides are designed for mutagenesis by introduction of an altered nucleotide at the desired position of the genome or antigenome. The plasmid containing the genetically altered genome or antigenome is then amplified.

Mutations can vary from single nucleotide changes to the introduction, deletion or replacement of large cDNA segments containing one or more genes or genome segments. Genome segments can correspond to structural and/or functional domains, e.g., cytoplasmic, transmembrane or ectodomains of proteins, active sites such as sites that mediate binding or other biochemical interactions with different proteins, epitopic sites, e.g., sites that stimulate antibody binding and/or humoral or cell mediated immune responses, etc. Useful genome segments in this regard range from about 15-35 nucleotides in the case of genome segments encoding small functional domains of proteins, e.g., epitopic sites, to about 50, 75, 100, 200-500, and 500-1,500 or more nucleotides.

In addition to these polynucleotide sequence relationships, proteins and protein regions encoded by recombinant HPIV1 of the invention are also typically selected to have conservative relationships, i.e. to have substantial sequence identity or sequence similarity, with selected reference polypeptides. As applied to polypeptides, the term “sequence identity” means peptides share identical amino acids at corresponding positions. The term “sequence similarity” means peptides have identical or similar amino acids (i.e., conservative substitutions) at corresponding positions. The term “substantial sequence identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Abbreviations for the twenty naturally occurring amino acids used herein follow conventional usage (Immunology-A Synthesis, 2nd ed., E. S. Golub & D. R. Gren, eds., Sinauer Associates, Sunderland, Mass., 1991, incorporated herein by reference). Stereoisomers (e.g., D-amino acids) of the twenty conventional amino acids, unnatural amino acids such as α,α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). Moreover, amino acids may be modified by glycosylation, phosphorylation and the like.

To select candidate viruses according to the invention, the criteria of viability, attenuation and immunogenicity are determined according to well known methods. Viruses that will be most desired in immunogenic compositions of the invention must maintain viability, have a stable attenuation phenotype, exhibit replication in an immunized host (albeit at lower levels), and effectively elicit production of an immune response in a subject sufficient to elicit an immune response against wild-type virus. The recombinant HPIV1 viruses of the invention are not only viable and more appropriately attenuated than previous immunogenic agents, but are more stable genetically in vivo, retaining the ability to stimulate an immune response and in some instances to expand immunity afforded by multiple modifications, e.g., induce an immune response against different viral strains or subgroups, or by a different immunologic basis, e.g., secretory versus serum immunoglobulins, cellular immunity, and the like.

Recombinant HPIV1 viruses of the invention can be tested in various well known and generally accepted in vitro and in vivo models to confirm adequate attenuation, resistance to phenotypic reversion, and immunogenicity for use in immunogenic compositions. In in vitro assays, the modified virus (e.g., a multiply attenuated, biologically derived or recombinant PIV) is tested, e.g., for temperature sensitivity of virus replication, i.e., is phenotype, and for the small plaque or other desired phenotype. Modified viruses are further tested in animal models of PIV infection. A variety of animal models have been described. PIV model systems, including rodents and non-human primates, for evaluating attenuation and immunogenic activity of PIV candidates of the invention are widely accepted in the art, and the data obtained therefrom correlate well with PIV infection, attenuation and immunogenicity in humans.

In accordance with the foregoing description, the invention also provides isolated, infectious recombinant HPIV1 compositions for use in immunogenic compositions. The attenuated virus which is a component of an immunogenic composition is in an isolated and typically purified form. By isolated is meant to refer to PIV which is in other than a native environment of a wild-type virus, such as the nasopharynx of an infected individual. More generally, isolated is meant to include the attenuated virus as a component of a cell culture or other artificial medium where it can be propagated and characterized in a controlled setting. For example, attenuated HPIV1 of the invention may be produced by an infected cell culture, separated from the cell culture and added to a stabilizer.

For use in immunogenic compositions, recombinant HPIV1 produced according to the present invention can be used directly in formulations, or lyophilized, as desired, using lyophilization protocols well known to the artisan. Lyophilized virus will typically be maintained at about 4° C. When ready for use the lyophilized virus is reconstituted in a stabilizing solution, e.g., saline or comprising SPG, Mg⁺⁺ and HEPES, with or without adjuvant, as further described below.

HPIV1-based immunogenic compositions of the invention contain as an active ingredient an immunogenically effective amount of a recombinant HPIV1 produced as described herein. The modified virus may be introduced into a host with a physiologically acceptable carrier and/or adjuvant. Useful carriers are well known in the art, and include, e.g., water, buffered water, 0.4% saline, 0.3% glycine, hyaluronic acid and the like. The resulting aqueous solutions may be packaged for use as is, or lyophilized, the lyophilized preparation being combined with a sterile solution prior to administration, as mentioned above. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like. Acceptable adjuvants include incomplete Freund's adjuvant, MPL™ (3-o-deacylated monophosphoryl lipid A; RIBI ImmunoChem Research, Inc., Hamilton, Mont.) and IL-12 (Genetics Institute, Cambridge Mass.), among many other suitable adjuvants well known in the art.

Upon immunization with a recombinant HPIV1 composition as described herein, via aerosol, droplet, oral, topical or other route, the immune system of the host responds to the immunogenic composition by producing antibodies specific for PIV proteins, e.g., F and HN glycoproteins. As a result of the immunization with an immunogenically effective amount of a recombinant HPIV1 produced as described herein, the host becomes at least partially or completely immune to infection by the targeted PIV or non-PIV pathogen, or resistant to developing moderate or severe infection therefrom, particularly of the lower respiratory tract.

The host to which the immunogenic compositions are administered can be any mammal which is susceptible to infection by PIV or a selected non-PIV pathogen and which host is capable of generating an immune response to the antigens of the vaccinizing strain. Accordingly, the invention provides methods for creating immunogenic compositions for a variety of human and veterinary uses.

The compositions containing the recombinant HPIV1 of the invention are administered to a host susceptible to or otherwise at risk for PIV infection to enhance the host's own immune response capabilities. Such an amount is defined to be a “immunogenically effective dose.” In this use, the precise amount of recombinant HPIV1 to be administered within an effective dose will depend on the host's state of health and weight, the mode of administration, the nature of the formulation, etc., but will generally range from about 10³ to about 10⁷ plaque forming units (PFU) or 50% tissue culture infectious dose 50 (TCID₅₀), or more of virus per host, more commonly from about 10⁴ to 10⁶ PFU or TCID₅₀ virus per host. In any event, the immunogenic composition should provide a quantity of modified PIV of the invention sufficient to effectively elicit a detectable immune response in the subject.

The recombinant HPIV1 produced in accordance with the present invention can be combined with viruses of other PIV serotypes or strains to achieve immunization against multiple PIV serotypes or strains. Alternatively, immunization against multiple PIV serotypes or strains can be achieved by combining protective epitopes of multiple serotypes or strains engineered into one virus, as described herein. Typically when different viruses are administered they will be in a mixture and administered simultaneously, but they may also be administered separately. Immunization with one strain may elicit an immune response against different strains of the same or different serotype.

In some instances it may be desirable to combine the recombinant HPIV1 immunogenic compositions of the invention with immunogenic compositions that induce immune responses to other agents, particularly other childhood viruses. In another aspect of the invention the recombinant HPIV1 can be employed as a vector for protective antigens of other pathogens, such as respiratory syncytial virus (RSV) or measles virus, by incorporating the sequences encoding those protective antigens into the recombinant HPIV1 genome or antigenome which is used to produce infectious virus, as described herein.

In all subjects, the precise amount of recombinant HPIV1 immunogenic composition administered, and the timing and repetition of administration, will be determined based on the patient's state of health and weight, the mode of administration, the nature of the formulation, etc. Dosages will generally range from about 10³ to about 10⁷ plaque forming units (PFU) or TCID₅₀, or more of virus per patient, more commonly from about 10⁴ to 10⁶ PFU or TCID₅₀ virus per patient. In any event, the immunogenic compositions should provide a quantity of attenuated recombinant HPIV1 sufficient to effectively stimulate or induce an anti-PIV or other anti-pathogenic immune response, e.g., as can be determined by hemagglutination inhibition, complement fixation, plaque neutralization, and/or enzyme-linked immunosorbent assay, among other methods. In this regard, individuals are also monitored for signs and symptoms of upper respiratory illness. As with administration to chimpanzees, the attenuated virus grows in the nasopharynx at levels approximately 10-fold or more lower than wild-type virus, or approximately 10-fold or more lower when compared to levels of incompletely attenuated virus.

In neonates and infants, multiple administration may be required to elicit sufficient levels of immunity. Administration should begin within the first month of life, and at intervals throughout childhood, such as at two months, six months, one year and two years, as necessary to maintain sufficient levels of immunity against native (wild-type) PIV infection. Similarly, adults who are particularly susceptible to repeated or serious PIV infection, such as, for example, health care workers, day care workers, family members of young children, the elderly, individuals with compromised cardiopulmonary function, may require multiple immunizations to establish and/or maintain immune responses. Levels of induced immunity can be monitored by measuring amounts of neutralizing secretory and serum antibodies, and dosages adjusted or immunizations repeated as necessary to maintain desired levels of immunity. Further, different recombinant viruses may be indicated for administration to different recipient groups. For example, an engineered HPIV1 expressing a cytokine or an additional protein rich in T cell epitopes may be particularly advantageous for adults rather than for infants.

HPIV1-based immunogenic compositions produced in accordance with the present invention can be combined with viruses expressing antigens of another subgroup or strain of PIV to achieve an immune response against multiple PIV subgroups or strains. Alternatively, the immunogenic virus may incorporate protective epitopes of multiple PIV strains or subgroups engineered into one PIV clone, as described herein.

The recombinant HPIV1 immunogenic compositions of the invention elicit production of an immune response that alleviates serious lower respiratory tract disease, such as pneumonia and bronchiolitis when the individual is subsequently infected with wild-type PIV. While the naturally circulating virus is still capable of causing infection, particularly in the upper respiratory tract, there is a very greatly reduced possibility of rhinitis as a result of the immunization. Boosting of resistance by subsequent infection by wild-type virus can occur. Following immunization, there are detectable levels of host engendered serum and secretory antibodies which are capable of neutralizing homologous (of the same subgroup) wild-type virus in vitro and in vivo.

Preferred recombinant HPIV1 candidates of the invention exhibit a very substantial diminution of virulence when compared to wild-type virus that naturally infects humans. The virus is sufficiently attenuated so that symptoms of infection will not occur in most immunized individuals. In some instances the attenuated virus may still be capable of dissemination to unimmunized individuals. However, its virulence is sufficiently abrogated such that severe lower respiratory tract infections in the immunized or incidental host do not occur.

The level of attenuation of recombinant HPIV1 candidates may be determined by, for example, quantifying the amount of virus present in the respiratory tract of an immunized host and comparing the amount to that produced by wild-type PIV or other attenuated PIV which have been evaluated as candidate strains. For example, the attenuated virus of the invention will have a greater degree of restriction of replication in the upper respiratory tract of a highly susceptible host, such as a chimpanzee, compared to the levels of replication of wild-type virus, e.g., 10- to 1000-fold less. In order to further reduce the development of rhinorrhea, which is associated with the replication of virus in the upper respiratory tract, an ideal candidate virus should exhibit a restricted level of replication in both the upper and lower respiratory tract. However, the attenuated viruses of the invention must be sufficiently infectious and immunogenic in humans to elicit an immune response in immunized individuals. Methods for determining levels of PIV in the nasopharynx of an infected host are well known in the literature.

Levels of induced immunity provided by the immunogenic compositions of the invention can also be monitored by measuring amounts of neutralizing secretory and serum antibodies. Based on these measurements, dosages can be adjusted or immunizations repeated as necessary to maintain desired levels of immunity. Further, different viruses may be advantageous for different recipient groups. For example, an engineered recombinant HPIV1 strain expressing an additional protein rich in T cell epitopes may be particularly advantageous for adults rather than for infants.

In yet another aspect of the invention the recombinant HPIV1 is employed as a vector for transient gene therapy of the respiratory tract. According to this embodiment the recombinant HPIV1 genome or antigenome incorporates a sequence that is capable of encoding a gene product of interest. The gene product of interest is under control of the same or a different promoter from that which controls PIV expression. The infectious recombinant HPIV1 produced by coexpressing the recombinant HPIV1 genome or antigenome with the N, P, L and other desired PIV proteins, and containing a sequence encoding the gene product of interest, is administered to a patient. Administration is typically by aerosol, nebulizer, or other topical application to the respiratory tract of the patient being treated. Recombinant HPIV1 is administered in an amount sufficient to result in the expression of therapeutic or prophylactic levels of the desired gene product. Representative gene products that may be administered within this method are preferably suitable for transient expression, including, for example, interleukin-2, interleukin-4, gamma-interferon, GM-CSF, G-CSF, erythropoietin, and other cytokines, glucocerebrosidase, phenylalanine hydroxylase, cystic fibrosis transmembrane conductance regulator (CFTR), hypoxanthine-guanine phosphoribosyl transferase, cytotoxins, tumor suppressor genes, antisense RNAs, and viral antigens.

The following examples are provided by way of illustration, not limitation.

### Example 1

### rHPIV1-C^{R84G/Δ170}HN^{T553A}L^{Y942A} and rHPIV1-C^{R84G/Δ170}HN^{T553A}L^{Δ1710-11}

The present invention relates in its basic aspect to two HPIV1 vaccine candidate viruses, rHPIV1-C^{R84G/Δ170}HN^{T553A}L^{Y942A} and rHPIV1-C^{R84G/Δ170}HN^{T553A}L^{Δ1710-11}. The present Example and Example 2 following describe the characterization of these two viruses in vitro and in vivo. Each of the mutant viruses is named according to the mutations it contains: C^{F170S} refers to the indicated amino acid substitution in the C protein and confers a non-ts att phenotype; C^{Δ170} refers to a six-nucleotide deletion spanning codon 170 in C and confers a non-ts att phenotype; C^{R84G} and HN^{T553A} refer to amino acid substitutions in C and HN that, in combination, confer a non-ts att phenotype but individually have no attenuation phenotype; L^{Y942A} refers to the indicated amino substitution in L and confers a ts att phenotype; and L^{Δ1710-11} has the deletion of the indicated residues in L and confers a ts att phenotype. The C^{F170S} mutation is silent in the overlapping P protein. The rHPIV1-C^{F170S} mutant tested both here and in previous studies contains the non-attenuating HN^{T553A} mutation. Since previous studies have referred to this virus simply as rHPIV1-C^{F170S} we will employ the same nomenclature here for the purpose of comparison.

### Cells and Viruses

LLC-MK2 cells (ATCC CCL 7.1) and HEp-2 cells (ATCC CCL 23) were maintained in Opti-MEM I (Gibco-Invitrogen, Inc. Grand Island, N.Y.) supplemented with 5% FBS and gentamicin sulfate (50 μg/ml). Vero cells (ATCC CCL-81) were maintained in Opti-PRO SFM (Gibco-Invitrogen, Inc.) in the absence of FBS and supplemented with gentamicin sulfate (50 μg/ml) and L-glutamine (4 mM). BHK-T7 cells, which constitutively express T7 RNA polymerase (Buchholz U J, Finke S, Conzelmann K K: Generation of bovine respiratory syncytial virus (BRSV) from cDNA: BRSV NS2 is not essential for virus replication in tissue culture, and the human RSV leader region acts as a functional BRSV genome promoter. J. Virol. 1999, 73:251-259.), were kindly provided by Dr. Ulla Buchholz, NIAID, and were maintained in GMEM (Gibco-Invitrogen, Inc.) supplemented with 10% FBS, geneticin (1 mg/ml), MEM amino acids, and L-glutamine (2 mM). Biologically-derived wt HPIV1 Washington/20993/1964, the parent for the recombinant virus system, was isolated previously from a clinical sample in primary African green monkey kidney (AGMK) cells and passaged 2 additional times in primary AGMK cells (Murphy B R, Richman D D, Chalhub E G, Uhlendorf C P, Baron S, Chanock R M: Failure of attenuated temperature-sensitive influenza A (H3N2) virus to induce heterologous interference in humans to parainfluenza type 1 virus. Infect Immun. 1975, 12:62-68.) and once in LLC-MK2 cells (Bartlett E J, Amaro-Carambot E, Surman S R, Newman J T, Collins P L, Murphy B R, Skiadopoulos M H: Human parainfluenza virus type I (HPIV1) vaccine candidates designed by reverse genetics are attenuated and efficacious in African green monkeys. Vaccine 2005, 23:4631-4646.). This preparation has a wild type phenotype in AGMs, and will be referred to here as HPIV1 wt. It was previously described as HPIV1_{LLC1}. HPIV1 wt and rHPIV1 mutants were grown in LLC-MK2 cells in the presence of 1.2% Tryple select, a recombinant trypsin (Gibco-Invitrogen, Inc.), as described previously (Newman J T, Surman S R, Riggs J M, Hansen C T, Collins P L, Murphy B R, Skiadopoulos M H: Sequence analysis of the Washington/1964 strain of human parainfluenza virus type 1 (HPIV1) and recovery and characterization of wild-type recombinant HPIV1 produced by reverse genetics. Virus Genes 2002, 24:77-92.).

Construction of Mutant HPIV1 cDNA

P/C, HN and L gene mutations (Table 1) were introduced into the appropriate rHPIV1 subgenomic clones (Newman J T, Riggs J M, Surman S R, McAuliffe J M, Mulaikal T A, Collins P L, Murphy B R, Skiadopoulos M H: Generation of recombinant human parainfluenza virus type 1 vaccine candidates by importation of temperature-sensitive and attenuating mutations from heterologous paramyxoviruses. J. Virol. 2004, 78:2017-2028.) using the Advantage-HF PCR Kit (Clontech Laboratories, Palo Alto, Calif.) with a modified PCR mutagenesis protocol described elsewhere (Moeller K, Duffy I, Duprex P, Rima B, Beschorner R, Fauser S, Meyermann R, Niewiesk S, ter Meulen V, Schneider-Schaulies J: Recombinant measles viruses expressing altered hemagglutinin (H) genes: functional separation of mutations determining H antibody escape from neurovirulence. J. Virol. 2001, 75:7612-7620.). The entire PCR amplified subgenomic clone was sequenced using a Perkin-Elmer ABI 3100 sequencer with the Big Dye sequencing kit (Perkin-Elmer Applied Biosystems, Warrington, UK) to confirm that the subclone contained the introduced mutation and to confirm the absence of adventitious mutations introduced during PCR amplification. Full-length antigenomic cDNA clones (FLCs) of HPIV1 containing the desired mutations were assembled using standard molecular cloning techniques, and the region containing the introduced mutation in each FLC was sequenced as described above to confirm the presence of the introduced mutation and absence of adventitious changes. Each virus was designed to conform to the rule of six, which is a requirement by HPIV1 and numerous other paramyxoviruses that the nucleotide length of their genome be an even multiple of six for efficient replication.

Recovery of rHPIV1 Mutant Viruses

Three different recovery methods were used to generate rHPIV1 mutants that differed in the source of the T7 polymerase needed to synthesize RNA from the transfected virus-specific plasmids and, in one case, a different transfection method was used. First, using the procedure according to Newman et al. (Virus Genes 2002, 24:77-92), rHPIV1 virus was recovered from HEp-2 cells that were transfected with plasmids encoding the antigenome and N, P, and L support proteins and infected with an MVA-T7 vaccinia virus recombinant as a source of T7 polymerase. Second, Vero cells were grown to 80% confluency and transfection experiments were performed using the AMAXA Cell Line Nucleofector Kit V, according to manufacturer's directions (AMAXA, Koeln, Germany). Briefly, the cells were transfected with 5 μg each of the FLC and the pCL-Neo-BCI-T7 plasmid (expressing T7 polymerase under the control of a eukaryotic promoter), 0.2 μg each of the N and P, and 0.1 μg of the L support plasmids. The transfection mixture was removed after 24 h at 37° C., and cells were washed and overlaid with Opti-PRO with L-glutamine (4 mM) supplemented with 1.2% Tryple select. The cells and supernatant were transferred to LLC-MK2 cells in T25 cm² flasks (Corning, N.Y.) 7 days following transfection. Third, BHK-T7 cells constitutively expressing T7 polymerase (Buchholz U J, Finke S, Conzelmann K K: Generation of bovine respiratory syncytial virus (BRSV) from cDNA: BRSV NS2 is not essential for virus replication in tissue culture, and the human RSV leader region acts as a functional BRSV genome promoter. J. Virol. 1999, 73:251-259.) were grown to 90 to 95% confluence in six-well plates. The cells were transfected with 5 μg of the FLC, 0.8 μg each of the N and P, and 0.1 μg of the L support plasmids in a volume of 100 μl of Opti-MEM per well. Transfection was carried out with Lipofectamine 2000 (Invitrogen, Inc., Carlsbad, Calif.), according to the manufacturer's directions. The transfection mixture was removed after a 24 h incubation period at 37° C., and the cells were washed and maintained in GMEM. On day 2 following transfection, the media was supplemented with 1.2% trypsin, and the recovered virus was harvested on days 2-4. All viruses were amplified by passage on LLC-MK2 cells, and each was cloned by two successive rounds of terminal dilution using LLC-MK2 monolayers in 96-well plates (Costar, Corning Inc., Acton, Mass.). To confirm that the recovered rHPIV1 mutants contained the appropriate mutations and lacked adventitious mutations, viral RNA (vRNA) was isolated from infected cell supernatants using the Qiaquick vRNA kit (Qiagen Inc., Valencia, Calif.), reverse transcribed using the SuperScript First-Strand Synthesis System (Invitrogen, Inc., Carlsbad, Calif.) and amplified using the Advantage cDNA PCR Kit (Clontech Laboratories). Each viral genome was sequenced in its entirety.

### Evaluation of Recombinant HPIV1 Vaccine Candidates in a Multiple Cycle Growth Curve

The recombinant HPIV1 mutants were compared to HPIV1 wt on LLC-MK2 and Vero cells at 32° C. in a multiple cycle growth curve. Confluent monolayer cultures in 6-well plates were infected in triplicate at a multiplicity of infection (MOI) of 0.01 50%-tissue-culture-infectious-doses (TCID₅₀) per cell in media containing trypsin. The residual inoculum was withdrawn 2 h post infection as the day 0 sample and was replaced by medium with trypsin. On days 2, and 4-11 post-infection, the total medium supernatant was removed for virus quantitation and was replaced with fresh medium with trypsin. Supernatants containing virus were frozen at −70° C., and all samples were tested together for virus titer with endpoints identified by hemadsorption.

Characterization of the Temperature Sensitivity of the rHPIV1 Vaccine Candidates

The is phenotype for each mutant rHPIV1 virus was determined by comparing its level of replication to that of HPIV1 wt at 32° C. and at 1° C. increments from 35° C. to 40° C., as described in Skiadopoulos et al. (Vaccine 1999, 18:503-510). Briefly, each virus was serially diluted 10-fold in 96-well LLC-MK2 monolayer cultures in L-15 media (Gibco-Invitrogen, Inc.) containing trypsin with four replicate wells per plate. Replicate plates were incubated at the temperatures indicated above for seven days, and virus infected wells were detected by hemadsorption with guinea pig erythrocytes. The virus titer at each temperature was determined in three to sixteen separate experiments and is expressed as the mean log₁₀ TCID₅₀/ml. The mean titer at an elevated temperature was compared to the mean titer at 32° C., and the reduction in mean titer was determined. The shut-off temperature of an rHPIV1 mutant is defined as the lowest temperature at which the reduction in virus titer compared to its titer at 32° C. was 100-fold greater than the reduction in HPIV1 wt titer between the same two temperatures. A mutant is defined as having a ts phenotype if its shut-off temperature is ≦40° C.

### Evaluation of Replication of Viruses in AGMs and Efficacy Against Challenge

AGMs in groups of two to four animals at a time were inoculated intranasally (i.n.) and intratracheally (i.t.) with 10⁶ TCID₅₀ of either HPIV1 wt or mutant rHPIV1 in a 1 ml inoculum at each site. NP swab samples were collected daily from days 1 to 10 post-inoculation, and TL fluid samples were collected on days 2, 4, 6, 8 and 10 post-inoculation. The specimens were flash frozen and stored at −80° C. and were subsequently assayed in parallel. Virus present in the samples was titered in dilutions on LLC-MK2 cell monolayers in 96-well plates and an undiluted 100 μl aliquot was also tested in 24-well plates. These were incubated at 32° C. for 7 days. Virus was detected by hemadsorption, and the mean log₁₀ TCID₅₀/ml was calculated for each sample day. The limit of detection was 0.5 log₁₀ TCID₅₀/ml. The mean peak titer for each group was calculated using the peak titer for each animal, irrespective of the day of sampling. The mean sum of the virus titers for each group was calculated from the sum, calculated for each animal individually, of the virus titers on each day of sampling, up to day 10. The sum of the lower limit of detectability was 5.0 log₁₀ TCID₅₀/ml for NP swabs and 2.5 log₁₀ TCID₅₀/ml for TL samples.

On day 28 post-inoculation, the AGMs were challenged i.n. and i.t. with 10⁶ TCID₅₀ of HPIV1 wt in 1 ml at each site. NP swab and TL samples were collected for virus quantitation on days 2, 4, 6 and 8 post-challenge.

All animal studies were performed under protocol LID22, as approved by the National Institute of Allergy and Infectious Disease (NIAID) Animal Care and Use Committee (ACUC).

### Evaluation of Immune Responses in AGMs

Serum was collected from each monkey on days 0 and 28 post-immunization and on day 28 post-challenge (day 56 post-immunization). HPIV1 HAI antibody titers were determined at 21° C., as described by Clements et al. (J Clin Microbiol 1991, 29:1175-1182) using 0.5% v/v guinea pig erythrocytes and HPIV1 wt as the antigen. The HAI antibody titer was defined as the end-point serum dilution that inhibited hemagglutination and is expressed as the mean reciprocal log₂±standard error (SE).

### Statistical Analysis

The Prism 4 (GraphPad Software Inc., San Diego, Calif.) one-way ANOVA test, (Student-Newman-Keuls multiple comparison test) was used to assess statistically significant differences between data groups (P<0.05). The R software programme (The GNU Operating System; www.gnu.org) was used to perform a Spearman rank test to determine correlation between data sets.

Construction and Recovery of Mutant rHPIV1 Viruses

Point and deletion mutations in the P/C, HN and L genes that attenuate HPIV1 for replication in the respiratory tract of hamsters or AGMs are indicated in Table 1.

TABLE 1
Summary of the mutations introduced into the rHPIV1 genome^{a}.

| | | | | | | | # nt |
|---|---|---|---|---|---|---|---|
| | | | | | | | changes for |
| | | | nt changes | Type of | Codon | | reversion |
| Gene | Mutation^{b} | ORF | wt→mutant^{c} | mutation | position | Amino acid change | to wt |
| P/C | R84G | C | AGA→GGA | point | 84 | R → G | 1 |
| | | P | GAG→GGG | point | 87 | E → G | 1 |
| | Δ170^{d} | C | AGG GAT | deletion | 168-170 | RDF → S | 6 (insertions)^{d} |
| | | | TTC → AGC | | | (D deletion; 3 nt | |
| | | | | | | deletions in the | |
| | | | | | | flanking R-F codons | |
| | | | | | | results in a S | |
| | | | | | | substitution) | |
| | | P | GGA TTT → | deletion | 172-173 | GF deletion | 6 (insertions) |
| | | | deletion | | | | |
| HN | T553A | HN | ACC→GCC | point | 553 | T → A | 1 |
| L | Y942A^{e} | L | TAT → GCG | point | 942 | Y → A | 3^{e} |
| | Δ1710-11^{d} | L | GCT GAG → | deletion | 1710-11 | AE deletion | 6 (insertions)^{d} |
| | | | deletion | | | | |

^{a}HPIV1 strain Washington/1964, GenBank accession no. NC_003461.
^{b}The nomenclature used to describe each mutation indicates the wt amino acid, the codon position and the new amino acid, or the position of the deletion (Δ), with respect to the C, HN or L protein.
^{c}The nucleotides (nt) affected by substitution or deletion are shown underlined and in bold type.
^{d}Designed for increased genetic stability by use of a deletion. Deletions involved six nt to conform to the rule of six [20].
^{e}Designed for increased genetic stability by the use of a codon that differs by three nucleotides from codons yielding a wild type assignment.

The C^{R84G} mutation is a single nucleotide substitution mutation that affects both the P and C proteins and that results in amino acid substitutions of R84 to G in C, and E87 to G in P (Table 1). The C^{R84G} mutation is attenuating in the upper respiratory tract (URT) of AGMs, but only in the presence of the HN^{T553A} point mutation indicated in Table 1. The C^{R84G} and HN^{T553A} mutations are each based on single nucleotide substitutions (Table 1), and thus the att phenotype would be lost by reversion at either position. The C^{Δ170} deletion mutation in HPIV1 involves a six-nucleotide deletion, a length that was chosen to comply with the “rule of six”. This deletion results in a loss of two amino acids and substitution of a third at codon positions 168-170 in C(RDF to S), and a deletion of amino acids GF in P at codon positions 172-173 (Table 1). The changes in the C protein also would be present in the nested C′, Y1, and Y2 proteins. The Y942A mutation in L has three nucleotide changes in codon 942 and specifies a genetically and phenotypically stabilized ts att phenotype.

In the present study, the L^{Δ1710-11} deletion mutation in HPIV1 was created at a site that corresponds by sequence alignment to a ts att point mutation originally identified in BPIV3. Importation of this BPIV3 point mutation has previously been shown to attenuate HPIV2. Here, the L^{Δ1710-11} mutation contains a six-nucleotide deletion that results in a deletion of amino acids AE at codon positions 1710-11 of the L gene of HPIV1 (Table 1).

The mutations in Table 1 were introduced into the HPIV1 antigenomic cDNA individually or in combinations to yield the panel of rHPIV1 viruses listed in Table 2. These viruses were recovered following transfection of cDNAs into HEp-2, BHK-T7 or Vero cells and biologically cloned in LLC-MK2 cells, and each was sequenced in its entirety to confirm the presence of the engineered mutation(s) and the absence of adventitious mutations.

TABLE 2
Level of temperature sensitivity of replication of rHPIV1 mutants in vitro.

| | | Virus | Mean reduction (log₁₀) in virus titer ± S.E. | | | | | | |
|---|---|---|---|---|---|---|---|---|---|
| | | titer ± | at the indicated temperature compared to | | | | | | |
| | | S.E. at | 32° C.^{c} | | | | | | Shut-off |
| Virus^{a} | | 32° C.^{b} | 35° C. | 36° C. | 37° C. | 38° C. | 39° C. | 40° C. | (° C.)^{d} |
| 1 | HPIV1 wt | 7.7 ± 0.1 | 0.1 ± 0.1 | 0.1 ± 0.1 | 0.2 ± 0.1 | 0.7 ± 0.1 | 1.3 ± 0.1 | 3.0 ± 0.3 | — |
| 2^{e} | rHPIV1-C^{R84G} | 9.2 ± 0.4 | 0.4 ± 0.2 | 0.4 ± 0.6 | 0.8 ± 0.5 | 0.3 ± 0.4 | 1.8 ± 0.6 | 4.5 ± 0.9 | — |
| 3^{e} | rHPIV1-C^{R84G}HN^{T553A} | 7.8 ± 0.1 | -0.3 ± 0.2 | -0.3 ± 0.2 | -0.2 ± 0.2 | 0.1 ± 0.2 | 0.7 ± 0.2 | 2.5 ± 0.6 | — |
| 4^{e} | rHPIV1-C^{Δ170} | 7.9 ± 0.3 | 0.2 ± 0.2 | 0.7 ± 0.8 | 0.5 ± 0.2 | 1.0 ± 0.3 | 2.6 ± 0.7 | 4.5 ± 1.0 | — |
| 5 | rHPIV1-L^{Y942A} | 8.0 ± 0.1 | 0.2 ± 0.3 | 1.2 ± 0.3 | 2.6 ± 1.1^{c,d} | 6.4 ± 0.4 | ≧6.8^{f} | ≧6.8 | 37° C. |
| 6^{e} | rHPIV1- | 7.4 ± 0.2 | 0.4 ± 0.4 | 0.5 ± 0.4 | 2.3 ± 0.4 | 4.0 ± 0.6 | 6.0 ± 0.4 | ≧6.4 | 37° C. |
| | C^{R84G}HN^{T553A}L^{Y942A} | | | | | | | | |
| 7 | rHPIV1-C^{R84G}L^{Δ1710-11} | 7.5 ± 0.7 | 0.8 ± 0.7 | 3.0 ± 0.6 | 4.8 ± 0.2 | ≧6.3 | ≧6.3 | ≧6.3 | 36° C. |
| 8 | rHPIV1- | 6.3 ± 0.1 | 0.3 ± 0.2 | 0.9 ± 0.6 | 2.0 ± 0.3 | 4.9 ± 0.2 | ≧5.1 | ≧5.1 | 38° C. |
| | C^{R84G/Δ170}HN^{T553A}L^{Y942A} | | | | | | | | |
| 9 | rHPIV1- | 6.4 ± 0.3 | 2.6 ± 0.6 | 4.0 ± 0.4 | ≧5.2 | ≧5.2 | ≧5.2 | ≧5.2 | 35° C. |
| | C^{R84G/Δ170}HN^{T553A}L^{Δ1710-11} | | | | | | | | |

^{a}Data are the mean of three to sixteen experiments.
^{b}Viruses were titrated on LLC-MK2 cells at either permissive (32° C.) or potentially restrictive (35° C.-40° C.) temperatures for 7 days and virus titers are expressed as the mean ± standard error (S.E.). The limit of detection was 1.2 log₁₀ TCID₅₀/ml.
^{c}Values in bold indicate restricted replication, where the mean log₁₀ reduction in virus titer at the indicated temperature vs 32° C. was 2.0 log₁₀ or greater than the difference in titer of HPIV1 wt at the same temperature vs 32° C. A virus is designated ts if restricted replication at 35° C.-40° C. is observed.
^{d}Underlined values indicate viral shut-off temperature, the lowest temperature at which restricted replication is observed.
^{e}These data have been previously published and are included here for the purposes of comparison.
^{f}The symbol “≧” indicates that virus titers were at the limit of detection and therefore the reduction in virus titer versus 32° C. is greater than or equal to the indicated value. There is no S.E. value for viruses at the limit of detection.

Unexpectedly, rHPIV1 containing the L^{Δ1710-11} mutation by itself was unable to be isolated. However, virus bearing L^{Δ1710-11} in the presence of C^{R84G} without adventitious mutations was recovered. Thus, analysis of the phenotype of the L^{Δ1710-11} mutation was performed in the presence of the C^{R84G} mutation, which is neither is nor att.

Characterization of rHPIV1 s Containing Single att Mutations

We first sought to characterize the rHPIV1 mutants bearing the four single att mutations (the C^{R84G}HN^{T553A} set, C^{Δ170}, L^{Y942A}, and L^{Δ1710-11}) to define the contributions of the individual mutations to the phenotypes of the rHPIV1 mutants (Groups 3, 4, 5, 7 in Tables 2 and 3). We previously generated and evaluated the rHPIV1-C^{R84G}HN^{T553A} and rHPIV1-C^{Δ170} viruses (each containing a single non-ts att mutation) in vitro and in vivo (McAuliffe J M, Surman S R, Newman J T, Riggs J M, Collins P L, Murphy B R, Skiadopoulos M H: Codon substitution mutations at two positions in the L polymerase protein of human parainfluenza virus type 1 yield viruses with a spectrum of attenuation in vivo and increased phenotypic stability in vitro. J Virol 2004, 78:2029-2036; Bartlett E J, Amaro-Carambot E, Surman S R, Newman J T, Collins P L, Murphy B R, Skiadopoulos M H: Human parainfluenza virus type I (HPIV1) vaccine candidates designed by reverse genetics are attenuated and efficacious in African green monkeys. Vaccine 2005, 23:4631-4646; Bartlett E J, Amaro-Carambot E, Surman S R, Collins P L, Murphy B R, Skiadopoulos M H: Introducing point and deletion mutations into the P/C gene of human parainfluenza virus type 1 (HPIV1) by reverse genetics generates attenuated and efficacious vaccine candidates. Vaccine 2006, 24:2674-2684.)

These previously evaluated single-mutation viruses were included here for the purpose of comparison with viruses containing the other individual mutations as well as combinations of mutations. An rHPIV1 mutant, rHPIV1-L^{Y942A}, bearing the Y942A mutation in L was generated for the present study. We had previously generated and characterized a virus, rHPIV1-C^{R84G}HN^{T553A}L^{Y942A} containing the L^{Y942A} mutation in combination with the C^{R84G}HN^{T553A} pair of mutations (McAuliffe J M, Surman S R, Newman J T, Riggs J M, Collins P L, Murphy B R, Skiadopoulos M H: Codon substitution mutations at two positions in the L polymerase protein of human parainfluenza virus type 1 yield viruses with a spectrum of attenuation in vivo and increased phenotypic stability in vitro. J Virol 2004, 78:2029-2036.)

The newly generated rHPIV1-L^{Y942A} virus would permit evaluation of its specific contribution to the level of temperature sensitivity in vitro and attenuation in vivo. The rHPIV1 mutant bearing the individual att mutation L^{Δ1710-11} (rHPIV1-C^{R84G}L^{Δ1710-11}) also contained the C^{R84G} mutation, although this latter mutation is phenotypically silent on its own, as already noted.

TABLE 3
Level of replication of HPIV1 vaccine candidates in the upper and lower respiratory tract of African green monkeys.

| | | | | Mean peak | | | | | |
|---|---|---|---|---|---|---|---|---|---|
| | | | | virus titer | | Mean sum of the | | | |
| | | | | (log₁₀ | | daily virus titers | | | |
| | | | No. | TCID₅₀/ml)^{c} | | (log₁₀ TCID₅₀/ml)^{d} | | | |
| | | Shut-off | of | NP | | NP | | att^{e} | |
| Virus^{a} | | temperature^{b} | animals | swab^{f} | TL^{g} | swab^{f} | TL^{g} | URT | LRT |
| 1 | HPIV1 wt | — | 14 | 4.2 ± 0.2 | 3.9 ± 0.3 | 26.4 ± 1.5 | 12.2 ± 1.6 | — | — |
| 2^{h} | rHPIV1-C^{R84G} | — | 4 | 3.6 ± 0.4 | 4.0 ± 0.5 | 21.0 ± 1.7 | 11.7 ± 2.5 | No | No |
| 3^{h} | rHPIV1-C^{R84G}HN^{T553A} | — | 12 | 2.1 ± 0.2¹ | 4.8 ± 0.3 | 10.5 ± 0.9 | 14.3 ± 1.1 | Yes | No |
| 4^{h} | rHPIV1-C^{Δ170} | — | 6 | 3.4 ± 0.5 | 2.3 ± 0.5 | 14.8 ± 1.9 | 5.1 ± 0.8 | Yes | Yes |
| 5 | rHPIV1-L^{Y942A} | 37° C. | 4 | 2.3 ± 0.1 | 2.3 ± 0.2 | 16.9 ± 0.7 | 8.4 ± 1.2 | Yes | Yes |
| 6^{h} | rHPIV1- | 37° C. | 8 | 2.4 ± 0.2 | 2.1 ± 0.3 | 12.9 ± 1.0 | 5.1 ± 0.6 | Yes | Yes |
| | C^{R84G}HN^{T553A}L^{Y942A} | | | | | | | | |
| 7 | rHPIV1-C^{R84G}L^{Δ1710-11} | 36° C. | 4 | 1.5 ± 0.4 | 0.9 ± 0.2 | 8.6 ± 1.8 | 3.2 ± 0.6 | Yes | Yes |
| 8 | rHPIV1- | 38° C. | 4 | 1.2 ± 0.3 | 0.6 ± 0.1 | 5.9 ± 0.5 | 2.6 ± 0.1 | Yes | Yes |
| | C^{R84G/Δ170}HN^{T553A}L^{Y942A} | | | | | | | | |
| 9 | rHPIV1- | 35° C. | 4 | 0.9 ± 0.3 | ≦0.5 ± 0.0 | 6.3 ± 0.5 | ≦2.5 ± 0.0 | Yes | Yes |
| | C^{R84G/Δ170}HN^{T553A}L^{Δ1710-11} | | | | | | | | |

^{a}Monkeys were inoculated i.n. and i.t. with 10⁶ TCID₅₀ of the indicated virus in a 1 ml inoculum at each site. Data are representative of one to five experiments.
^{b}Shut-off temperature is defined in footnote d, Table 2.
^{c}Virus titrations were performed on LLC-MK2 cells at 32° C. and expressed as the mean ± S.E of the individual peak virus titers for the animals in each group irrespective of day. The limit of detection was 0.5 log₁₀ TCID₅₀/ml.
^{d}Mean sum of the daily virus titers: the sum of the titers for all of the days of sampling was determined for each animal individually, and the mean was calculated for each group. On days when virus was not detected, a value of was 0.5 log₁₀ TCID₅₀/ml was assigned for the purpose of calculation. The mean sum of the lower limit of detection was 5.0 log₁₀ TCID₅₀/ml for NP swabs and 2.5 log₁₀ TCID₅₀/ml for TL samples.
^{e}Virus is designated att in the URT or LRT based on a significant reduction in either mean peak titer or mean sum of daily titers compared to the HPIV1 wt group (see footnote h).
^{f}Nasopharyngeal (NP) swab samples were collected on days 1-10 post-infection.
^{g}Tracheal lavage (TL) samples were collected on days 2, 4, 6, 8, and 10 post-infection.
^{h}These data have been previously published and are included here for the purposes of comparison.

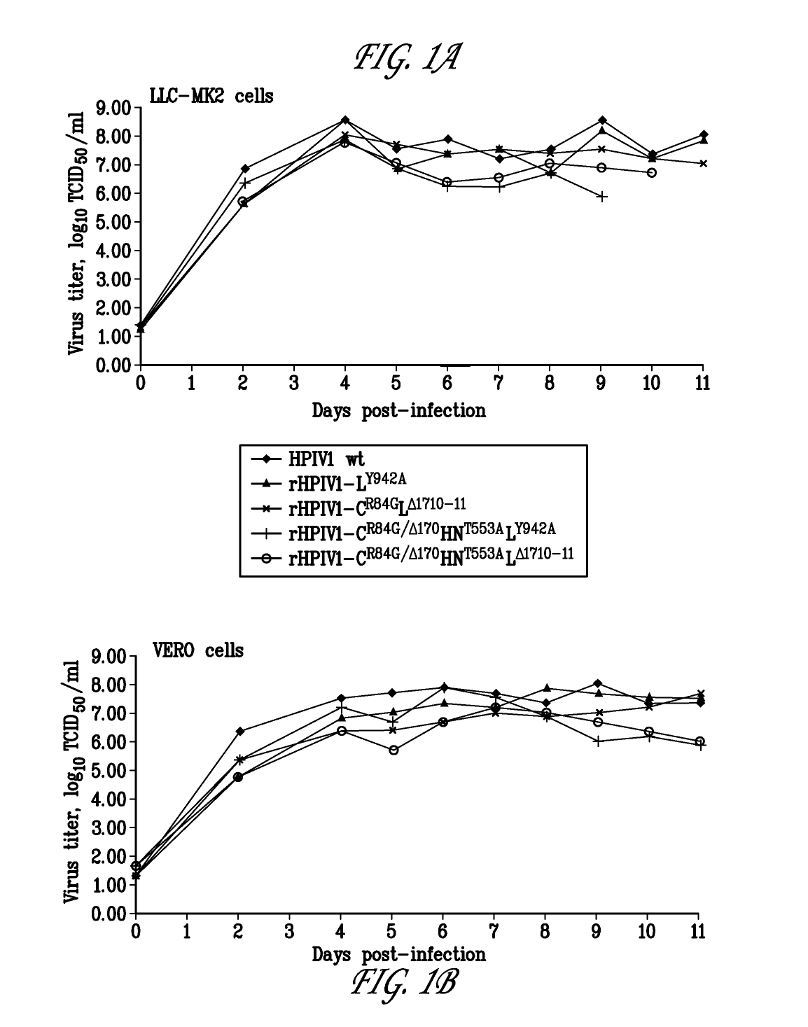

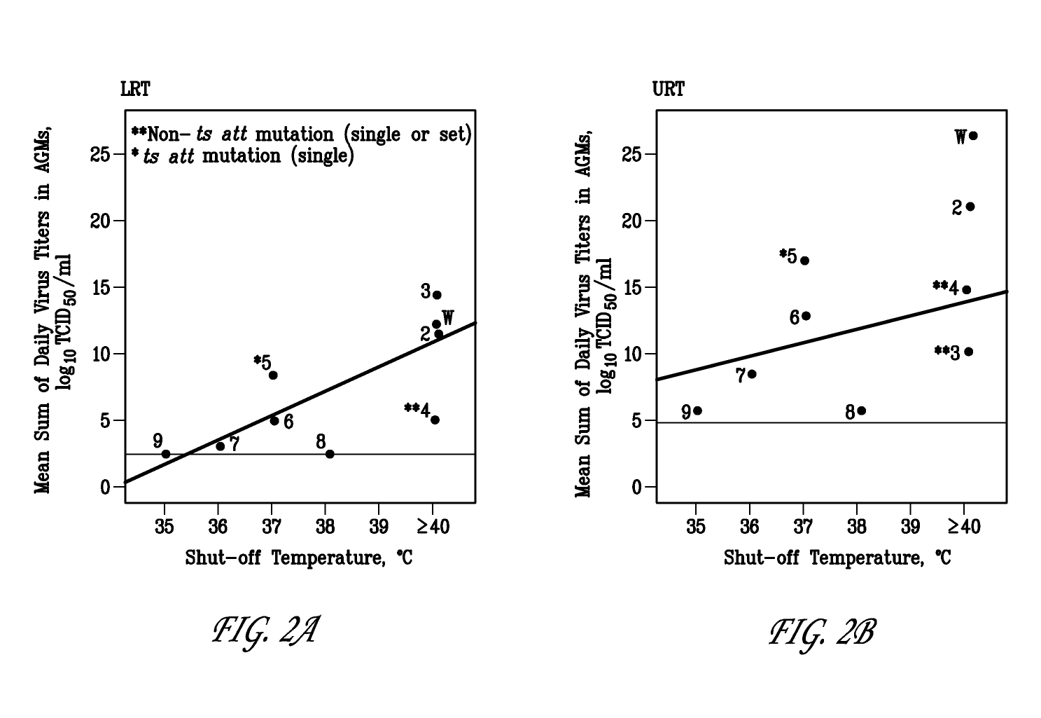

The level of temperature sensitivity of replication of the four viruses with single att mutations was first studied (Table 2, groups 3, 4, 5, and 7) and compared to that of rHPIV1 wt and rHPIV1-C^{R84G}. Viruses containing only P/C gene mutations with or without the HN mutation were non-ts, whereas each of the L gene mutations specified a ts phenotype in vitro. The single L^{Y942A} mutation specified a shut-off temperature of 37° C., a level of temperature sensitivity that was equivalent to that previously observed for rHPIV1-C^{R84G}HN^{T553A}L^{Y942A} (Table 2, compare Groups 5 and 6). These data indicate that the L^{Y942A} mutation is responsible for the observed ts phenotype of rHPIV1-C^{R84G} HN^{T553A}L^{Y942A}(Table 2). The L^{Δ1710-11} mutation specified an even stronger ts phenotype than the L^{Y942A} mutation (Table 2). The L^{AΔ1710-11} mutation clearly contributes significantly to the ts property of rHPIV1-C^{R84G}L^{Δ1710-11} since rHPIV1-C^{R84G} was confirmed to be non-ts (Table 2, compare Groups 2 and 7). Therefore, both L^{Y942A} and L^{Δ1710-11} are ts mutations in HPIV1. In a multiple cycle growth curve, the two newly generated rHPIV1 mutants with single att mutations, rHPIV1-L^{Y942A} and rHPIV1-C^{R84G}L^{Δ1710-11}, reached a titer equivalent to that of rHPIV1 wt in both LLC-MK2 and Vero cells. FIG. 1 presents the comparison of the replication of HPIV1 wt and rHPIV1 mutant viruses containing the indicated mutations in the P/C, HN and L genes in a multiple cycle growth curve. Monolayer cultures of LLC-MK2 cells and Vero cells were infected at a multiplicity of infection of 0.01 TCID₅₀/cell and incubated at 32° C. The medium was removed on days 0 (residual inoculum), 2 and 4-11 post-infection, frozen for later determination of virus titers, and replaced by fresh medium containing trypsin. The virus titers shown are the means of 3 replicate cultures. Thus, these individual mutations do not significantly restrict replication in vitro at the permissive temperature of 32° C. and therefore could be useful mutations in vaccine candidates.

The level of replication of rHPIV1-L^{Y942A} and rHPIV1-C^{R84G}L^{Δ1710-11} in AGMs was next evaluated and compared to that of rHPIV1 wt and the other two single att mutants (Table 3, Groups 1, 3, 4, 5, 7). A rHPIV1 mutant was considered attenuated if it exhibited a significant (P<0.05) reduction in replication in either the mean peak virus titer or the mean sum of the daily virus titers (a measure of the total amount of virus shed over the duration of the infection) in either the nasopharyngeal (NP) swab (representative of the upper respiratory tract, URT) or tracheal lavage (TL) samples (representative of the lower respiratory tract, LRT) compared to the HPIV1 wt group. We have previously demonstrated that rHPIV1-C^{R84G} replicates to levels equivalent to HPIV1 wt in AGMs, whereas rHPIV1-C^{R84G} HN^{T553A} and rHPIV1-C^{R84G}HN^{T553A}L^{Y942A} were attenuated in AGMs. Here, both rHPIV1-L^{Y942A} and rHPIV1-C^{R84G}L^{Δ1710-11} were significantly attenuated in the URT and LRT of AGMs in comparison to HPIV1 wt. The levels of attenuation of rHPIV1-L^{Y942A} and rHPIV1-C^{R84G}HN^{T553A}L^{Y942A} were comparable, indicating that the L^{Y942A} mutation is an attenuating mutation by itself and that the attenuation specified by the L^{Y942A} mutation is not additive to that specified by the C^{R84G}HN^{T553A} att mutation. The rHPIV1-C^{R84G}L^{Δ1710-11} mutant also was significantly attenuated in AGMs, reducing virus titer in comparison to HPIV1 wt by 2.7 and 3.0 log₁₀ 50%-tissue-culture-infectious-doses (TCID₅₀)/ml in the URT and LRT, respectively (Table 3). Since rHPIV1-C^{R84G} was confirmed not to be attenuated in AGMs (Table 3, Group 2), this suggests that the L^{Δ1710-11} mutation contributes significantly to the observed attenuation phenotype.

The immunogenicity and protective efficacy resulting from immunization with rHPIV1s containing single att mutations were evaluated in AGMs by measuring post-immunization HPIV1 hemagglutination inhibiting (HAI) serum antibody titers and by challenging immunized and control animals with HPIV1 wt 28 days following immunization and determining challenge virus titers in the URT and LRT (Table 4). AGMs immunized with rHPIV1s containing single att mutations (Groups 3, 4, 5, and 7) developed post-immunization HAI serum antibodies and manifested resistance to replication of the challenge virus. The rHPIV1-C^{R84G}L^{Δ1710-11} mutant, which showed a strong level of attenuation following immunization of AGMs, was protective only at a low level in the URT.

TABLE 4
Section 1.11 Table 4. Immunogenicity and protective efficacy of rHPIV1 vaccine candidates in AGMs.

| | | | | | | Mean sum of | | |
|---|---|---|---|---|---|---|---|---|
| | | | | Mean peak | | the daily | | |
| | | | | challenge | | challenge | | |
| | | | Pre- | virus titer | | virus titers | | |
| | | | challenge | (log₁₀ | | (log₁₀ | | Post- |
| | | | serum | TCID₅₀/ml)^{c} | | TCID₅₀/ml)^{d} | | challenge |
| | | No. | HAI | NP | | NP | | serum |
| Virus^{a} | | animals | titer^{b} | swab | TL | swab | TL | HAI titer^{b} |
| 1 | HPIV1 wt | 12 | 6.7 ± 0.6 | 0.8 ± 0.2^{f} | 0.7 ± 0.1 | 2.3 ± 0.2 | 2.4 ± 0.2 | 6.6 ± 0.5 |
| | | | (12/12) | | | | | |
| 2^{e} | rHPIV1-C^{R84G} | 4 | 3.8 ± 0.9 | ≦0.5 ± 0.0 | ≦0.5 ± 0.0 | ≦2.0 ± 0.0 | ≦2.0 ± 0.0 | 4.4 ± 1.2 |
| | | | (3/4) | | | | | |
| 3^{e} | rHPIV1-C^{R84G}HN^{T553A} | 12 | 6.0 ± 0.6 | 0.6 ± 0.1 | 0.6 ± 0.1 | 2.1 ± 0.1 | 2.1 ± 0.1 | 7.9 ± 0.4 |
| | | | (11/12) | | | | | |
| 4^{e} | rHPIV1-C^{Δ170} | 6 | 5.5 ± 0.4 | ≦0.5 ± 0.0 | ≦0.5 ± 0.0 | ≦2.0 ± 0.0 | ≦2.0 ± 0.0 | 6.5 ± 0.4 |
| | | | (6/6) | | | | | |
| 5 | rHPIV1-L^{Y942A} | 4 | 6.3 ± 1.2 | 1.1 ± 0.2 | 1.2 ± 0.2 | 2.7 ± 0.3 | 2.8 ± 0.3 | 8.9 ± 1.1 |
| | | | (4/4) | | | | | |
| 6^{e} | rHPIV1- | 8 | 2.0 ± 0.0 | 0.8 ± 0.2 | 0.8 ± 0.2 | 2.6 ± 0.3 | 2.4 ± 0.3 | 3.3 ± 0.7 |
| | C^{R84G}HN^{T553A}L^{Y942A} | | (3/8) | | | | | |
| 7 | rHPIV1-C^{R84G}L^{Δ1710-11} | 4 | 6.1 ± 1.8 | 3.4 ± 0.6 | 3.0 ± 0.6 | 8.4 ± 2.0 | 8.3 ± 1.3 | 6.9 ± 1.5 |
| | | | (3/4) | | | | | |
| 8 | rHPIV1- | 4 | ≦1.0 ± 0.0 | 2.2 ± 0.2 | 1.8 ± 0.5 | 5.1 ± 0.3 | 4.3 ± 1.3 | 5.5 ± 1.6 |
| | C^{R84G/Δ170}HN^{T553A}L^{Y942A} | | (0/4) | | | | | |
| 9 | rHPIV1- | 4 | ≦1.0 ± 0.0 | 4.5 ± 0.9 | 3.4 ± 0.4 | 11.8 ± 2.5 | 8.1 ± 1.3 | 7.5 ± 1.4 |
| | C^{R84G/Δ170}HN^{T553A}L^{Δ1710-11} | | (0/4) | | | | | |
| 10 | Non-immune | 7 | ≦1.0 ± 0.0 | 5.0 ± 0.6 | 3.9 ± 0.5 | 14.8 ± 1.2 | 11.0 ± 2.5 | 6.0 ± 1.3 |
| | | | (0/4) | | | | | |

^{a}Monkeys were immunized i.n. and i.t. with 10⁶ TCID₅₀ of the indicated virus in a 1 ml inoculum at each site and were challenged on day 28 post-infection with HPIV1 wt.
^{b}HAI titers to HPIV1 were determined by HAI assay of sera collected at day 28 (pre-challenge) and day 56 (post-challenge) in separate assays. Titers are expressed as mean reciprocal log₂ ± S.E.; the limit of detection was 1.0 ± 0.0. The number of animals with a 4-fold or greater increase in pre-challenge antibody titers is shown in brackets for each group.
^{c}Mean ± S.E of the individual peak virus titers for the animals in each group irrespective of day. Virus titrations were performed on LLC-MK2 cells at 32° C. The limit of detection was 0.5 log₁₀ TCID₅₀/ml. NP and TL samples were collected on days 2, 4, 6 and 8 post-challenge.
^{d}Mean sum of the daily virus titers: the sum of the titers for all of the days of sampling was determined for each animal individually, and the mean was calculated for each group. On days when no virus was detected, a value of was 0.5 log₁₀ TCID₅₀/ml was assigned for the purpose of calculation. The mean sum of the lower limit of detection was 2.0 log₁₀ TCID₅₀/ml for NP swabs and TL samples.
^{e}These data have been previously published and are included here for the purposes of comparison.
^{f}Underlined values indicate statistically significant reductions in mean peaks or sum of daily virus titers for HPIV1 wt titer compared to the corresponding non-immune group, P < 0.05 (Student-Newman-Keuls multiple comparison test).

Combination of Three Single att Mutations into rHPIV1 to Generate Two Live Attenuated HPIV1 Vaccine Candidates

Having identified the in vitro and in vivo properties of the four single att mutations, information was used to generate two live attenuated HPIV1 vaccine candidates containing both non-ts and ts attenuating mutations. These vaccine candidates were designed to incorporate a backbone containing one stabilized non-ts attenuating mutation, C^{Δ170}, as well as the C^{R84G}HN^{T553A} att mutation. The addition of this second mutation (the C^{R84G}HN^{T553A} att mutation) was expected to increase the overall stability of the virus by increasing the total number of attenuating mutations present in the vaccine candidate. To generate the two live attenuated HPIV1 vaccine candidates, either the stabilized ts att L^{Y942A} mutation or the L^{Δ1710-11} deletion mutation was added to the rHPIV1-C^{R84G/Δ170}HN^{T553A} backbone. The resulting combination mutants, rHPIV1-C^{R84G/Δ170}HN^{T553A}L^{Y942A} and rHPIV1-C^{R84G/Δ170}HN^{T553A}L^{Δ1710-11}, were then evaluated as potential vaccine candidates.

These two viruses were first evaluated for their level of temperature sensitivity of replication in vitro (Table 2). The level of temperature sensitivity of rHPIV1-C^{R84G/Δ170}HN^{T553A}L^{Y942A} and rHPIV1-C^{R84G/Δ170}HN^{T553A}L^{Δ1710-11} (Groups 8 and 9 in Table 2) was equivalent to that of the corresponding L gene single-mutation viruses from which they were derived (namely rHPIV1-L^{Y942A} and rHPIV1-C^{R84G}L^{Δ1710-1}, Groups 5 and 7 in Table 2). This indicates that combining the non-ts and ts mutations in rHPIV1-C^{R84G/Δ170} HN^{T553A}L^{Y942A} and rHPIV1-C^{R84G/Δ170}HN^{T553A}L^{Δ1710-11} did not significantly alter their overall level of temperature sensitivity of replication in vitro. A multiple cycle growth curve at 32° C. demonstrated that each virus achieved titers in Vero cells that will allow efficient manufacture. Specifically, the rHPIV1-C^{R84G/Δ170}HN^{T553A}L^{Y942A} and rHPIV1-C^{R84G/Δ170}HN^{T553A}L^{Δ1710-11} vaccine candidates reached peak titers of 7.9 and 7.2 log₁₀ TCID₅₀/ml, respectively, in Vero cells (FIG. 1).

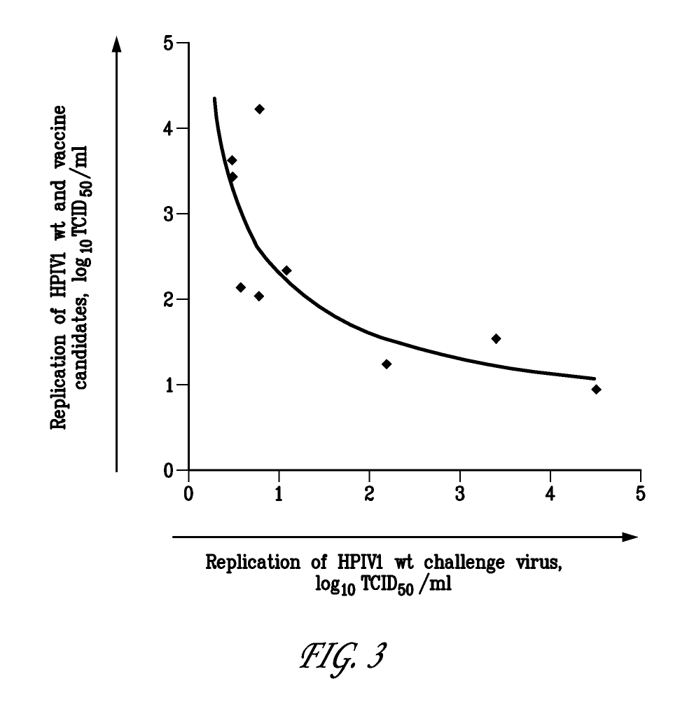

The level of replication of rHPIV1-C^{R84G/Δ170}HN^{T553A}L^{Y942A} and rHPIV1-C^{R84G/Δ170}HN^{T553A}L^{Δ1710-11} in AGMs was next evaluated and compared to that of rHPIV1 wt and the other two single att mutants (Table 3, Groups 1, 3, 4, 5, 7, 8, and 9). The rHPIV1-C^{R84G/Δ170}HN^{T553A}L^{Y942A} virus was strongly attenuated compared to rHPIV1 mutants bearing the corresponding single att mutations only in C/P, C/P/HN or L. The mean peak titer of rHPIV1-C^{R84G/Δ170}HN^{T553A}L^{Y942A} in the URT and LRT was reduced by 3.0 and 3.3 log₁₀ TCID₅₀/ml, respectively, in comparison to HPIV1 wt (Table 3). Similarly, the addition of the HN^{T553A} and C^{Δ170} mutations to rHPIV1-C^{R84G}L^{Δ1710-11} to generate the rHPIV1 C^{R84G/Δ170}HN^{T553A}L^{Δ1710-11} further attenuated the virus in AGMs, restricting virus replication in comparison to HPIV1 wt by 3.1 and 3.4 log₁₀ TCID₅₀/ml in the URT and LRT, respectively (Table 3). Therefore these two HPIV1 vaccine candidates demonstrate strong attenuation phenotypes in vivo. Considering the 9 viruses in Table 3 together, a relationship was found to exist between level of temperature sensitivity of replication in vitro and the attenuation manifested in vivo, i.e., the lower the shut off temperature, the higher the level of in vivo attenuation (FIG. 2). Evaluation of these data using the Spearman rank test gives correlation coefficients of 0.47 and 0.67 for the URT and LRT, respectively, based on the mean daily sum of virus titers for individual AGMs. This indicates a moderate positive correlation with a stronger association between the level of temperature sensitivity and virus replication in the LRT. However, as might be expected, viruses bearing only the non-ts attenuating P/C gene mutations, including the C^{Δ170} and the C^{R84G}HN^{T553A} set of mutations, did not follow this pattern (FIG. 2), and a higher correlation coefficient would be expected if these non-ts viruses were not included in the analysis. FIG. 2 presents the representation of the association between the in vitro shut-off temperature and the attenuation phenotype in AGMs for HPIV1 wt (W) and rHPIV1 mutant viruses. Here, for each virus (number designations correspond to the virus group numbers assigned in tables 2-4), the shut-off temperature (° C.), as determined by an in vitro temperature sensitivity assay (Table 2), was plotted against the mean sum of daily virus titers (log₁₀ TCID₅₀/ml; Table 3) in the URT (A) and LRT (B) of AGMs. rHPIV1 wt and non-ts rHPIV1 mutants were assigned a shut-off temperature of 40° C. for the purposes of this schematic. The limit of detection for the mean sum of daily virus titers is shown by a dashed line and viruses containing a single or set of non-ts attenuating mutation (**) or a single is attenuating mutation (*) are highlighted, as shown. A linear trend line fit using the individual daily data is shown (solid line). The Spearman rank-correlation coefficient was determined to be 0.47 for the URT and 0.67 for the LRT, indicating a moderate positive correlation between shut-off temperature and mean daily sum of virus titer in the URT and a stronger association for the LRT.

The levels of immunogenicity and protective efficacy against HPIV1 wt challenge following immunization with rHPIV1-C^{R84G/Δ170}HN^{T553A}L^{Y942A} and rHPIV1-C^{R84G/Δ170}HN^{T553A}L^{Δ1710-11} were also determined (Groups 8 and 9 in Table 4). The two vaccine candidates failed to induce detectable HAI antibodies. However, immunization with the rHPIV1-C^{R84G/Δ170}HN^{T553A}L^{Y942A} was protective against HPIV1 wt challenge in both the URT and LRT (Table 4). In contrast, immunization with rHPIV1-C^{R84G/Δ170}HN^{T553A}L^{Δ1710-11} did not offer significant protection against HPIV1 wt challenge in the AGMs (Table 4), i.e., it appeared overattenuated in this animal model. A relationship was found between the level of replication of the immunizing virus and its ability to induce resistance to replication of the challenge virus (Tables 3 and 4), and this is graphically displayed in FIG. 3. Here, the mean peak virus titer (log₁₀ TCID₅₀/ml) in the URT following immunization (y-axis) was plotted for viruses 1-9 (Table 3) against the mean peak challenge virus titers (log₁₀ TCID₅₀/ml; x-axis) in the same groups (Table 4). A curve of best fit has been inserted (solid line) to demonstrate the association between these two data sets.

Live attenuated rHPIV1 vaccines have a number of advantages over inactivated or subunit vaccines, including the ability to: (i) induce the full spectrum of protective immune responses including serum and local antibodies as well as CD4+ and CD8+ T cells (Murphy B R: Mucosal immunity to viruses. In Mucosal Immunology, Second edition. Edited by Ogra P L, Mestecky, J., Lamm, M. E., Strober, W., McGhee, J. R., Bienstock, J.: Academic Press, Inc; 1999: 695-707.); (ii) infect and replicate in the presence of maternal antibody permitting immunization of young infants (Wright P F, Karron R A, Belshe R B, Thompson J, Crowe Jr J E, Boyce T G, Halburnt L L, Reed G W, Whitehead S S, Anderson E L, et al: Evaluation of a live, cold-passaged, temperature-sensitive, respiratory syncytial virus vaccine candidate in infancy. J Infect Dis 2000, 182:1331-1342; Karron R A, Belshe R B, Wright P F, Thumar B, Burns B, Newman F, Cannon J C, Thompson J, Tsai T, Paschalis M, et al.: A live human parainfluenza type 3 virus vaccine is attenuated and immunogenic in young infants. Pediatr Infect Dis J 2003, 22:394-405; (iii) cause an acute, self-limited infection that is readily eliminated from the respiratory tract; and (iv) replicate to high titers in cell substrates acceptable for products for human use, including qualified Vero cells, making manufacture of these vaccines commercially feasible. Two new rHPIV1 viruses containing att mutations in L, L^{Δ1710-11} and L^{Y942A}, were generated and characterized, and these is att mutations were used in combination with previously described non-ts att mutations in the P/C gene and HN gene to generate two new live attenuated HPIV1 vaccine candidates.

The creation of the L^{Δ1710-11} mutation was found to specify a strong ts att phenotype. The L^{Δ1710-11} mutation was originally identified as an attenuating point mutation, L^{T1711I}, in BPIV3 (Skiadopoulos M H, Schmidt A C, Riggs J M, Surman S R, Elkins W R, St Claire M, Collins P L, Murphy B R: Determinants of the host range restriction of replication of bovine parainfluenza virus type 3 in rhesus monkeys are polygenic. J Virol 2003, 77:1141-1148.). It was evaluated as a deletion mutation in the present study since a deletion mutation offers a higher level of genetic stability than a point mutation, a property that is desirable for mutations in a vaccine candidate. Indeed, since this deletion occurs in an ORF (in which the triplet nature of the codons must be maintained) and in a virus that conforms to the rule of six (in which the hexamer organization must be maintained), same-site reversion would require the precise restoration of six nucleotides. Unfortunately, a rHPIV1 mutant with only the L^{Δ1710-11} mutation was not able to be isolated since each rHPIV1-L^{Δ1710-11} mutant that was isolated also possessed one or more adventitious mutations. The L^{Δ1710-11} mutation could only be recovered free of adventitious mutations when it was in combination with the C^{R84G} mutation, and thus had to be studied in that context. rHPIV1-C^{R84G}L^{Δ1710-11} manifested a shut-off temperature of 36° C. in vitro and was restricted in replication in the URT and LRT of AGMs by 2.5 log₁₀ or 3.0 log₁₀, respectively (Table 3). Therefore, the L^{Δ1710-11} deletion mutation specifies a ts att phenotype for HPIV1, and, as such, is a suitable mutation to include in a HPIV1 vaccine candidate.

The L^{Y942A} mutation was identified previously as an attenuating mutation for introduction into potential HPIV1 vaccine candidates and was stabilized by codon optimization studies. These studies demonstrated that only three amino acids were shown to specify a wild type phenotype at this codon position (the wild type tyrosine, cysteine and phenylalanine) all of which would require three nucleotide changes to convert the GCG alanine to a codon specifying the wild type phenotype codon in the vaccine virus. In addition, the L^{Y942A} mutation was shown to be highly stable under selective pressure during passage at permissive and restrictive temperatures. Previous studies have evaluated the L^{Y942A} mutation only in the presence of the C^{R84G}HN^{T553A} set of mutations that attenuates HPIV1 for AGMs (McAuliffe J M, Surman S R, Newman J T, Riggs J M, Collins P L, Murphy B R, Skiadopoulos M H: Codon substitution mutations at two positions in the L polymerase protein of human parainfluenza virus type 1 yield viruses with a spectrum of attenuation in vivo and increased phenotypic stability in vitro. J Virol 2004, 78:2029-2036.)

To determine the specific contribution of the L^{Y942A} mutation to the ts and att phenotypes associated with the rHPIV1-C^{R84G}HN^{T553A}L^{Y942A} virus, a rHPIV1 containing only the L^{Y942A} mutation was generated and was found to be as attenuated as rHPIV1-C^{R84G}HN^{T553A}L^{Y942A} for AGMs. This indicated that the L^{Y942A} mutation independently attenuated HPIV1 for AGMs and can be used in the absence of the C^{R84G}HN^{T553A} mutation to attenuate HPIV1 for AGMs. The attenuation specified by the C^{R84G}HN^{T553A} mutation was not additive with that of L^{Y942A}. This actually is a desirable property, since it permits the inclusion of a greater number of mutations while avoiding over-attenuation, and these additional mutations would become unmasked in the case of the loss of one or more other mutations and would thus maintain the att phenotype. Thus, L^{Y942A} is a stable mutation that specifies a ts att phenotype for HPIV1 and is suitable for introducing into a HPIV1 vaccine candidate as an independent attenuating mutation.

The L^{Y942A} and L^{Δ1710-11} ts att mutations were used in conjunction with two of the non-ts att mutations, the C^{R84G}HN^{T553A} and C^{Δ170} mutations, to develop two live attenuated vaccine candidates for HPIV1, namely, rHPIV1-C^{R84G/Δ170}HN^{T553A}L^{Y942A} and rHPIV1-C^{R84G/Δ170}HN^{T553A}L^{Δ1710-11}. These vaccine candidates thus each contain three independent attenuating mutations (two non-ts att and one ts att mutation), two of which have been genetically stabilized. The combination of mutations present in these two vaccine candidates enhance the genetic and phenotypic stability of the viruses.

Evaluation of the two vaccine candidates revealed both candidates replicated well in Vero cells (FIG. 1), a characteristic that is important for manufacturing purposes. Both viruses also demonstrated a strong ts phenotype in vitro (shut-off temperature of ≦38° C.) that was similar to that of their ts parent virus (Table 2), but the two viruses differ in their level of temperature sensitivity in vivo. The HPIV1 vaccine candidates were both strongly attenuated in the URT and LRT of AGMs, with rHPIV1-C^{R84G/Δ170}HN^{T553A}L^{Y942A} replicating to slightly higher levels than the more ts rHPIV1-C^{R84G/Δ170}HN^{T553A}L^{Δ1710-11} (Table 3).

Compared to the T1711I point mutation in the L gene of BPIV, the Δ1710-1711 mutation is somewhat more attenuated in culture. The deletion mutation produces about a three degree lower “shut-off” temperature in a temperature sensitivity assay (compare rHPIV1-C^{R84G} and rHPIV-C^{R84G}L^{Δ1710-1711} in Table 2 above. However, the point mutation produces only a one degree lower “shut-off” temperature (data in Table 1 of M. Skiadopoulos et al., Determinants of the Host Range Restriction of Replication of Bovine Parainfluenza Virus Type 3 in Rhesus Monkeys are Polygenic, J. Virol. 2003, pp. 1141-1148). This greater degree of attenuation by the deletion mutation would not have been expected by one of skill in the art. rHPIV1-C^{R84G} L^{Δ1710-1711} grows well in culture; reaching a titer comparable to that of the wild type virus at a permissive temperature. Therefore, the combination of mutations C^{R84G} and L^{Δ1710-1711} is useful from a manufacturing viewpoint. The vaccine candidate strain rHPIV1-C^{R84G/Δ170}HN^{T553A} L^{Δ1710-1711} grows to a titer only a little more than 10-fold lower than wild type (Table 2), and thus is also a strain that is easy to culture for vaccine manufacture.

In vivo, the L^{Δ1710-1711} mutation appears to be a bit more attenuating than the T1711I point mutation previously described. A similar approximately 100-fold attenuation is seen in the URT of AGMs for both mutations, but the L^{Δ1710-1711} mutation is about 1000-fold attenuated in the LRT of AGMs, compared to about 100-fold attenuation observed for the T1711I mutation. (Table 3 herein and Table 2 of Skiadopoulous et al., et al., Determinants of the Host Range Restriction of Replication of Bovine Parainfluenza Virus Type 3 in Rhesus Monkeys are Polygenic, J. Virol. 2003, pp. 1141-1148.) This result is unexpected and suggests that the deletion mutation might be more useful in some vaccine constructs.

The above described quadruply-mutant viruses include a combination of ts and non-ts attenuating mutations. Compared to the originally characterized T1711I point mutation in the L protein, the Δ1710-1711 deletion mutation much more attenuating in combination with the other three mutations, even more so than the Y942A mutation in the L protein. The combination of mutations present in rHPIV1-C^{R84G/Δ170}HN^{T553A}L^{Y942A} and rHPIV1-C^{R84G/Δ170}HN^{T553A}L^{Δ1710-11} unexpected provide viruses that are highly attenuated in vivo yet maintain high levels of replication in culture, especially in Vero cells, thus providing for ease of manufacturing of a virus that causes little or no symptoms in individuals immunized with the virus.

Both vaccines were weakly immunogenic and failed to induce a detectable level of serum HAI antibodies in AGMs. A low level of protective efficacy was observed in AGMs immunized with rHPIV1-C^{R84G/Δ170}HN^{T553A}L^{Y942A}, but the rHPIV1-C^{R84G/Δ170}HN^{T553A}L^{Δ1710-11} was not protective. It is likely that these viruses will be more immunogenic, and therefore more efficacious, in humans compared to AGMs since they should replicate more efficiently in humans. HPIV1 is a human virus and it should replicate more efficiently in its natural host in which it causes disease than in AGMs in which it causes only an asymptomatic infection. These vaccine candidates are also highly ts and should replicate more efficiently in humans, which have a lower body core temperature (36.7° C.), than in AGMs (approximately 39° C.). The rHPIV1-C^{R84G/Δ170}HN^{T553A}L^{Y942A} and rHPIV1-C^{R84G/Δ170}HN^{T553A}L^{Δ1710-11} vaccine candidates are moving into clinical trials.

### Example 2

### HAE Model for HPIV1 Infection

The level of replication, cell tropism, and gross pathogenic effects of HPIV1 wt infection in HAE in an experimental setting (including low MOI infection and multi-cycle growth) that mimics virus infection of the lower conducting cartilaginous airways of humans was evaluated. The ability of HPIV1 wt and rHPIV1-C^{F170S} mutant viruses to induce a type 1 IFN response was also evaluated, and the role of the induced IFN in restricting replication of HPIV1 in HAE cultures examined. In this way, the phenotypes previously associated with HPIV1 C mutants in cell culture and in vivo in African green monkeys (AGMs) were characterized in HAE cultures. These data suggested that the HAE cells are predictive for HPIV1 infection and growth in vivo. Therefore, the level of attenuation of replication of two HPIV1 vaccine candidates in the airways of AGMs (Bartlett, E. J., E. Amaro-Carambot, S. R. Surman, P. L. Collins, B. R. Murphy, and M. H. Skiadopoulos. 2006. Introducing point and deletion mutations into the P/C gene of human parainfluenza virus type 1 (HPIV1) by reverse genetics generates attenuated and efficacious vaccine candidates. Vaccine 24:2674-2684; Bartlett, E. J., E. Amaro-Carambot, S. R. Surman, J. T. Newman, P. L. Collins, B. R. Murphy, and M. H. Skiadopoulos. 2005. Human parainfluenza virus type I (HPIV1) vaccine candidates designed by reverse genetics are attenuated and efficacious in African green monkeys. Vaccine 23:4631-46; McAuliffe, J. M., S. R. Surman, J. T. Newman, J. M. Riggs, P. L. Collins, B. R. Murphy, and M. H. Skiadopoulos. 2004. Codon substitution mutations at two positions in the L polymerase protein of human parainfluenza virus type 1 yield viruses with a spectrum of attenuation in vivo and increased phenotypic stability in vitro. J Virol 78:2029-36; Newman, J. T., J. M. Riggs, S. R. Surman, J. M. McAuliffe, T. A. Mulaikal, P. L. Collins, B. R. Murphy, and M. H. Skiadopoulos. 2004. Generation of recombinant human parainfluenza virus type 1 vaccine candidates by importation of temperature-sensitive and attenuating mutations from heterologous paramyxoviruses. J Virol 78:2017-28.) were compared to that seen in the HAE model. This comparison revealed that the level of attenuation of the vaccine candidates is similar in HAE cells and in AGMs, and in addition, unexpectedly revealed the ability of the HAE system to detect an attenuating effect of mutations in genes such as L that are not revealed by other in vitro cell culture systems. Clinical studies for one of the vaccine candidates, rHPIV1-C^{R84G/Δ170}HN^{T553A}L^{Y942A}, are currently in progress.

### Cells and Viruses

Human airway tracheobronchial epithelial cells were isolated from airway specimens from patients without underlying lung disease, provided by the National Disease Research Interchange (NDRI, Philadelphia, Pa.) or as excess tissue following lung transplantation under University of North Carolina at Chapel Hill (UNC) Institutional Review Board-approved protocols by the UNC Cystic Fibrosis Center Tissue Culture Core. Primary cells derived from single patient sources were expanded on plastic to generate passage 1 cells and plated at a density of 3×10⁵ cells per well on permeable Transwell-Col (12-mm diameter) or 2×10⁵ cells per well on permeable Millicell (12-mm diameter) supports. HAE cultures were grown in custom media with provision of an ALI for 4 to 6 weeks to form differentiated, polarized cultures that resemble in vivo pseudostratified mucociliary epithelium, as previously described (Pickles, R. J., D. McCarty, H. Matsui, P. J. Hart, S. H. Randell, and R. C. Boucher. 1998. Limited entry of adenovirus vectors into well-differentiated airway epithelium is responsible for inefficient gene transfer. Journal of virology 72:6014-23.). LLC-MK2 cells (ATCC CCL 7.1) and HEp-2 cells (ATCC CCL 23) were maintained in Opti-MEM I (Gibco-Invitrogen, Inc., Grand Island, N.Y.) supplemented with 5% FBS and gentamicin sulfate (50 μg/ml). Vero cells (ATCC CCL-81) were maintained in Opti-PRO SFM (Gibco-Invitrogen, Inc.) supplemented with 50 μg/ml gentamicin sulfate and 4 mM L-glutamine. Media used for HPIV1 propagation and infection in LLC-MK2 cells contained 1.2% TrypLESelect, a recombinant trypsin (Gibco-Invitrogen, Inc.), without FBS, in order to activate the HPIV1 F protein.

Biologically-derived wt HPIV1 Washington/20993/1964, the parent for the recombinant virus, was isolated previously from a clinical sample in primary AGM kidney (AGMK) cells and passaged 2 additional times in primary AGMK cells and once in LLC-MK2 cells. This preparation has a wild type phenotype in AGMs, was previously described as HPIV1_{LLC1} (Bartlett, E. J., E. Amaro-Carambot, S. R. Surman, J. T. Newman, P. L. Collins, B. R. Murphy, and M. H. Skiadopoulos. 2005. Human parainfluenza virus type I (HPIV1) vaccine candidates designed by reverse genetics are attenuated and efficacious in African green monkeys. Vaccine 23:4631-46.), and will be referred to here as HPIV1 wt or its recombinant version, rHPIV1 wt. The construction of the rHPIV1 mutants, rHPIV1-C^{F170S}, rHPIV1-C^{R84G/Δ170}HN^{T553A}L^{Y942A} and rHPIV1-C^{R84G/Δ170} HN^{T553A}L^{Δ1710-11}, was described previously (Bartlett, E. J., E. Amaro-Carambot, S. R. Surman, P. L. Collins, B. R. Murphy, and M. H. Skiadopoulos. 2006. Introducing point and deletion mutations into the P/C gene of human parainfluenza virus type 1 (HPIV1) by reverse genetics generates attenuated and efficacious vaccine candidates. Vaccine 24:2674-2684; Bartlett, E. J., A. Castano, S. R. Surman, P. L. Collins, M. H. Skiadopoulos, and B. R. Murphy. 2007. Attenuation and efficacy of human parainfluenza virus type 1 (HPIV1) vaccine candidates containing stabilized mutations in the P/C and L genes. Virol J 4:67.). Purified virus stocks were obtained by infecting LLC-MK2 cells and purifying the supernatant by centrifugation and banding in discontinuous 30/60% (w/v) sucrose gradients, steps designed to minimize contamination by cellular factors, especially IFN. Purification also removes exogenous trypsin from the virus preparation, however, since the viruses were prepared in trypsin media, it is likely that the F proteins of the inoculum virus were cleaved. The vesicular stomatitis virus (VSV) used was a recombinant VSV-GFP, originally obtained from John Hiscott (Stojdl, D. F., B. D. Lichty, B. R. tenOever, J. M. Paterson, A. T. Power, S. Knowles, R. Marius, J. Reynard, L. Poliquin, H. Atkins, E. G. Brown, R. K. Durbin, J. E. Durbin, J. Hiscott, and J. C. Bell. 2003. VSV strains with defects in their ability to shutdown innate immunity are potent systemic anti-cancer agents. Cancer Cell 4:263-75.). Stocks of VSV were propagated in Vero cells and sucrose purified, as indicated above.

Virus titers in samples were determined by 10-fold serial dilution of virus in 96-well LLC-MK2 monolayer cultures, using two to four wells per dilution. After 7 days at 32° C., infected cultures were detected by hemadsorption with guinea pig erythrocytes, as described previously (Skiadopoulos, M. H., T. Tao, S. R. Surman, P. L. Collins, and B. R. Murphy. 1999. Generation of a parainfluenza virus type 1 vaccine candidate by replacing the HN and F glycoproteins of the live-attenuated PIV3 cp45 vaccine virus with their PIV1 counterparts. Vaccine 18:503-10.). Virus titers are expressed as log₁₀ 50% tissue culture infectious dose per ml (log₁₀ TCID₅₀/ml). VSV stock titers were determined by plaque assay on Vero cells under 0.8% methyl cellulose overlay.

### Viral Inoculation of HAE

HAE cultures were washed with PBS to remove apical surface secretions and fresh media was supplied to the basolateral compartments prior to infection. HPIV1s were applied to the apical surface of HAE for inoculation at a low input MOI (0.01 TCID₅₀/cell) or high MOI (5.0 TCID₅₀/cell), and VSV was applied to the basolateral surface at an MOI of 4.2 PFU/cell, in a 100 μl inoculum. The inoculum was removed 2 h post-inoculation at either 32° C. or 37° C. The cells were then washed once for 5 min with PBS and incubated at 32° C. or 37° C., as indicated. Samples were harvested from the apical or basolateral surfaces of HAE for determination of virus titer or amount of type I IFN produced. Apical samples were collected by incubating the apical surface with 300 μl of media for 30 min at 32° C. or 37° C., after which the remaining fluid was recovered. Basolateral samples were collected directly from the basolateral compartment, and the volume removed was replaced with fresh media. Samples were stored at −80° C. prior to analysis.

### Histology and Immunostaining of Paraffin-Embedded Sections

HAE cultures were fixed in 4% paraformaldehyde (PFA) overnight and embedded in paraffin, and 5 μm histological sections were prepared. Sections were then either stained with hematoxylin and eosin (H&E) for analysis by light microscopy or were subjected to standard immunofluorescence protocols. Briefly, sections were blocked with 3% bovine serum albumin (BSA) in PBS++ (containing 1 mM CaCl₂ and 1 mM MgCl₂) and incubated with primary antibodies diluted in 1% BSA. Primary antibodies included a 1:4000 dilution of rabbit anti-HPIV1 obtained from fluid present in subcutaneous chambers of rabbits immunized with purified HPIV1 (HAI titer=1:2048), as described previously (8), and mouse anti-acetylated alpha tubulin (1: 2000, Zymed, San Francisco, Calif.). Secondary antibodies used were fluorescein isothiocyanate (FITC)-conjugated goat anti-rabbit IgG (Jackson ImmunoResearch Laboratories, Inc., West Grove, Pa.), and Texas Red-conjugated goat anti-mouse IgG (Jackson ImmunoResearch Laboratories, Inc.). After a final wash, cells were overlaid with VectaShield mounting medium (Vector Laboratories, Inc., Burlingame, Calif.). Images were acquired using a Leica DMIRB inverted fluorescence microscope equipped with a cooled-color charge-coupled device digital camera (MicroPublisher; Q-Imaging, Burnaby, BC, Canada).

### En Face Staining and Confocal Microscopy

HAE cultures were fixed overnight in 4% PFA and permeabilized with 2.5% triton-X 100. They were then blocked with 3% BSA/PBS++ and apical surfaces incubated with primary antibodies diluted in 1% BSA. An additional primary antiserum used for en face staining was a rabbit anti-HPIV1 polyclonal antiserum obtained by vaccinating whiffle-ball implanted rabbits. The primary rabbit anti-HPIV1 serum were used at a 1:100 dilution and the mouse anti-acetylated alpha tubulin antibodies were used at a dilution of 1:500. Secondary antibodies were FITC-conjugated goat anti-rabbit IgG and Alexa fluor 594 goat anti-mouse IgG (Molecular Probes). Fluorescent confocal XY optical sections were obtained using a Zeiss 510 Meta laser scanning confocal microscope.

### Type I IFN Bioassay

The amount of type I IFN produced by infected HAE was determined by an IFN bioassay following published methods (41). Briefly, clarified cell culture medium supernatants were treated at pH 2.0 for 24 h at 4° C. to inactivate virus and acid-labile type II IFN, and the pH was adjusted to 7.0 by the addition of 2M NaOH. Type I IFN concentrations were determined by measuring restriction of replication of VSV-GFP on HEp-2 cell monolayers in comparison to a known concentration of a human IFN-β standard (AVONEX; Biogen, Inc., Cambridge, Mass.). IFN-β standard (5000 pg/ml) and IFN-containing samples were serially diluted 10-fold in duplicate in 96-well plates of HEp-2 cells. After 24 h, the cells were washed and infected with VSV-GFP at 6.5×10⁴ PFU/well. Control cultures (no VSV-GFP, no IFN and VSV-GFP, no IFN) were performed in quadruplicate on each plate. After an additional 24 h, plates were read for total GFP expression on a Typhoon phosphorimager using a Typhoon 8600 scanner (Molecular Dynamics Inc., Sunnyvale, Calif.) control program (settings: fluorescence; filter, 526-SP green fluorescein). The dilution at which the level of GFP expression was approximately 50% of that in untreated cultures was determined as the end-point. The end-point of the AVONEX standard was compared to the end-point of the unknown samples, and IFN concentrations were determined and expressed as mean±SE (pg/ml). According to the manufacturers, using the World Health Organization natural IFN-β standard, the AVONEX IFN-β has a specific activity of approximately 1 IU of antiviral activity per 5 pg.

IFN mRNA qRT-PCR

The levels of IFN-α and IFN-β mRNA in HAE cultures infected with HPIV1 wt or rHPIV1 mutants relative to mock-infected cultures were determined by qRT-PCR, as previously described (51). Briefly, total intracellular RNA was extracted from cell cultures using the RNeasy total RNA isolation kit (QIAGEN, Valencia, Calif.). RNA was reverse transcribed using an oligo (dT) primer and reagents from the Brilliant qRT-PCR kit (Stratagene, La Jolla, Calif.). The PCR primers and Taqman probes used to detect human IFN-β, two specific sets of human IFN-α, and β-actin have been described previously (Spann, K. M., K. C. Tran, B. Chi, R. L. Rabin, and P. L. Collins. 2004. Suppression of the induction of alpha, beta, and lambda interferons by the NS1 and NS2 proteins of human respiratory syncytial virus in human epithelial cells and macrophages. J Virol 78:4363-9.). IFN-β primer set 1 was specific for human IFN-β1, -6 and -13 and IFN-α set 2 was specific for human IFN-α4, -5, -8, -10, -14, -17 and -21. Duplex q-PCR reactions were performed to allow comparison between IFN and the housekeeping gene, β-actin. The probe for β-actin was labeled with the reporter dye 5-carboxyfluorescein (FAM) at the 5′ end, and all IFN probes were labeled with the reporter dye 5′ HEX at the 5′ end and BHQ1 at the 3′ end. Reactions contained a passive reference dye, which was not involved in amplification of IFN or β-actin, but was used to normalize the probe reporter signals. Positive control curves were generated using preparations known to contain high levels of IFN cDNA to ensure reaction efficiency. Each reaction signal was corrected individually for β-actin signal. In addition, signals from all reactions from virus infected samples were corrected against the signal generated in a mock-infected well, resulting in a β-actin-corrected measurement of fold expression over mock.

Characterization of rHPIV1 wt Replication in HAE Cultures

It was previously shown that infection of HAE with RSV and HPIV3 was specific to the ciliated cells of the surface epithelium and was not associated with overt cytotoxicity, whereas infection with influenza A virus resulted in complete destruction of HAE cultures within 48 hr. In the present experiments, both the ability of HPIV1 wt to infect HAE and the response of the culture to infection were examined.

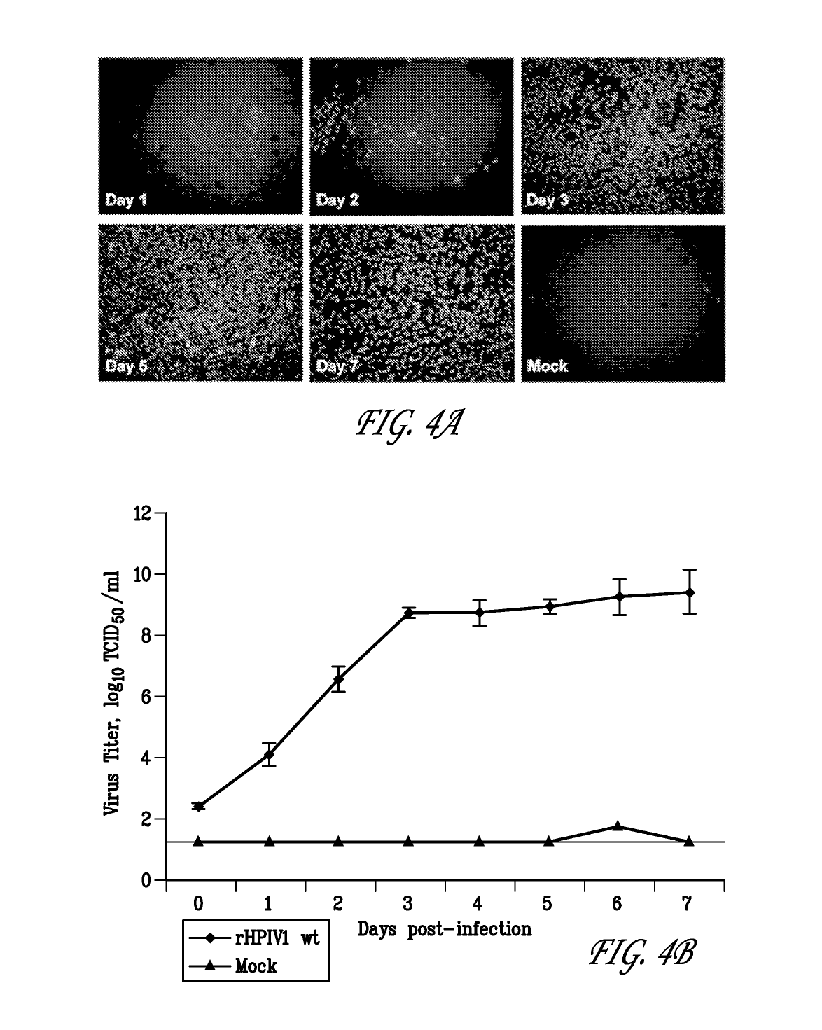

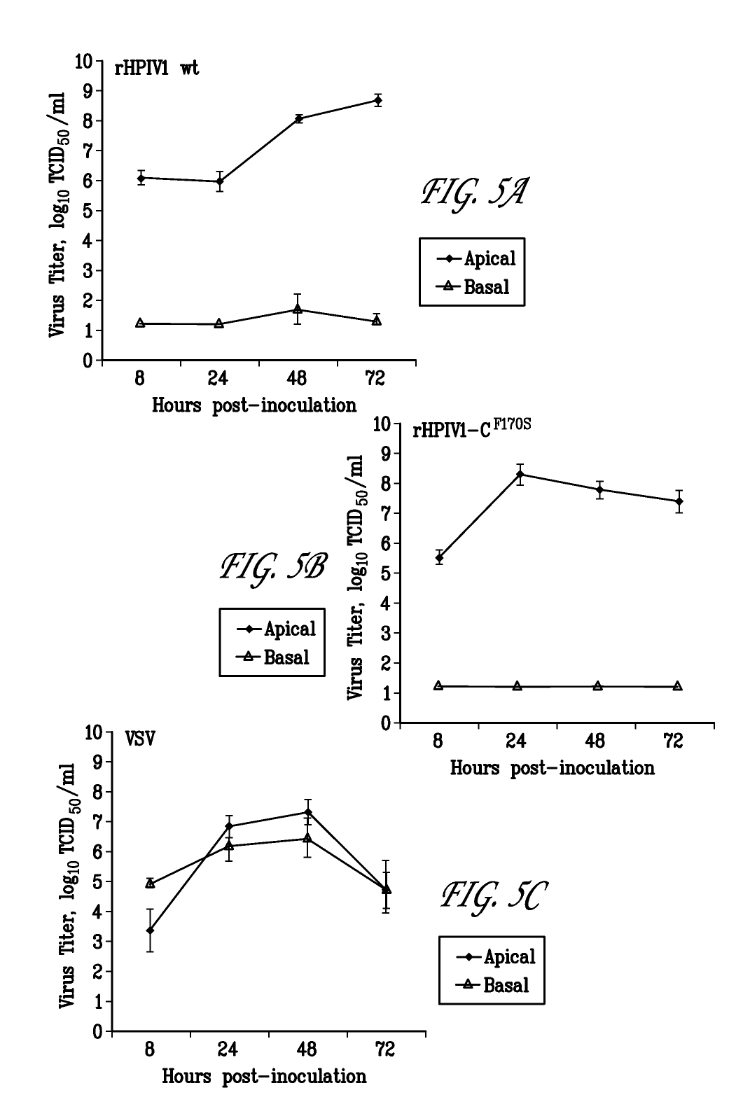

rHPIV1 wt efficiently infected HAE cells following apical inoculation with rHPIV1 wt at low MOI (0.01 TCID₅₀/cell). FIG. 4 indicates HPIV1 wt infects HAE cells, spreads throughout the culture and replicates efficiently. Here, HAE cells were mock-infected or infected with rHPIV1 wt at low MOI (0.01 TCID₅₀/cell). At days 1-7 p.i., (A) cells were fixed and stained en face for HPIV1 antigen (green), and (B) virus titers were determined in the apical compartments. Virus titers shown are the means of 3-11 cultures from a single donor±S.E. The limit of detection is 1.2 log₁₀TCID₅₀/ml, as indicated by the dashed line.

HPIV1 wt could be detected in HAE cells by en face immunostaining for HPIV1 antigen (FIG. 4A), and the increase in apical wash titers from day 0 to day 1 p.i. provided evidence of active replication and secretion of rHPIV1 wt (FIG. 4B). By day 3 p.i., virus had efficiently spread throughout the culture, with significant numbers of cells stained positive for HPIV1 through day 7 p.i. (FIG. 4A). Virus titers correlated with the numbers of cells staining positive for viral antigen in the en face immunostaining (FIG. 4).

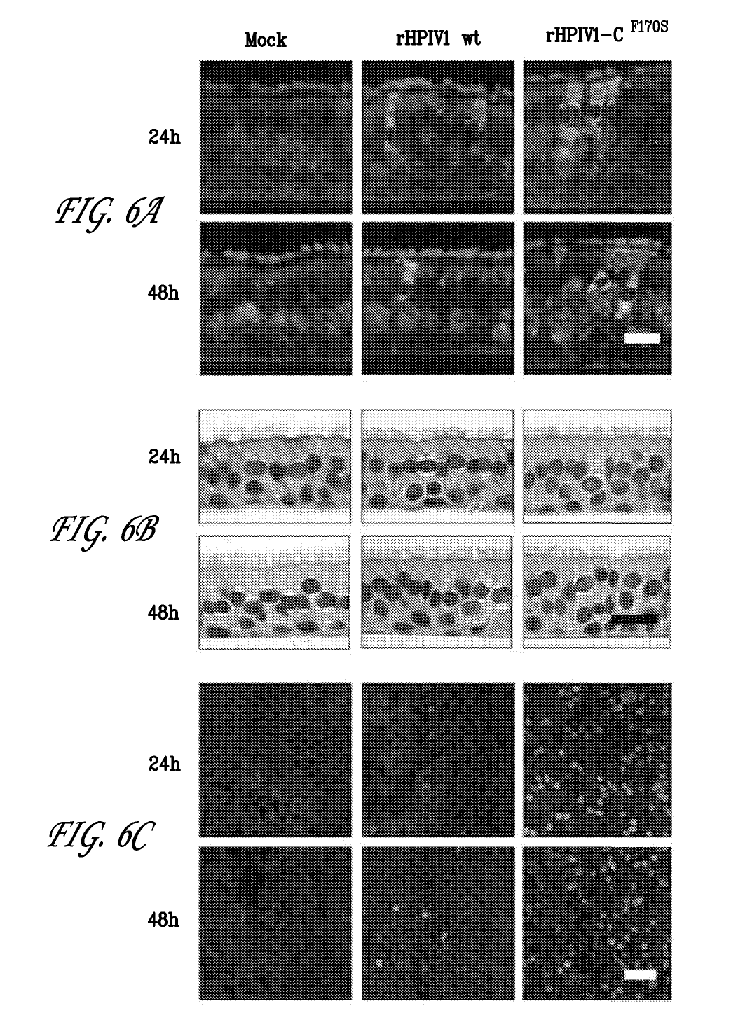

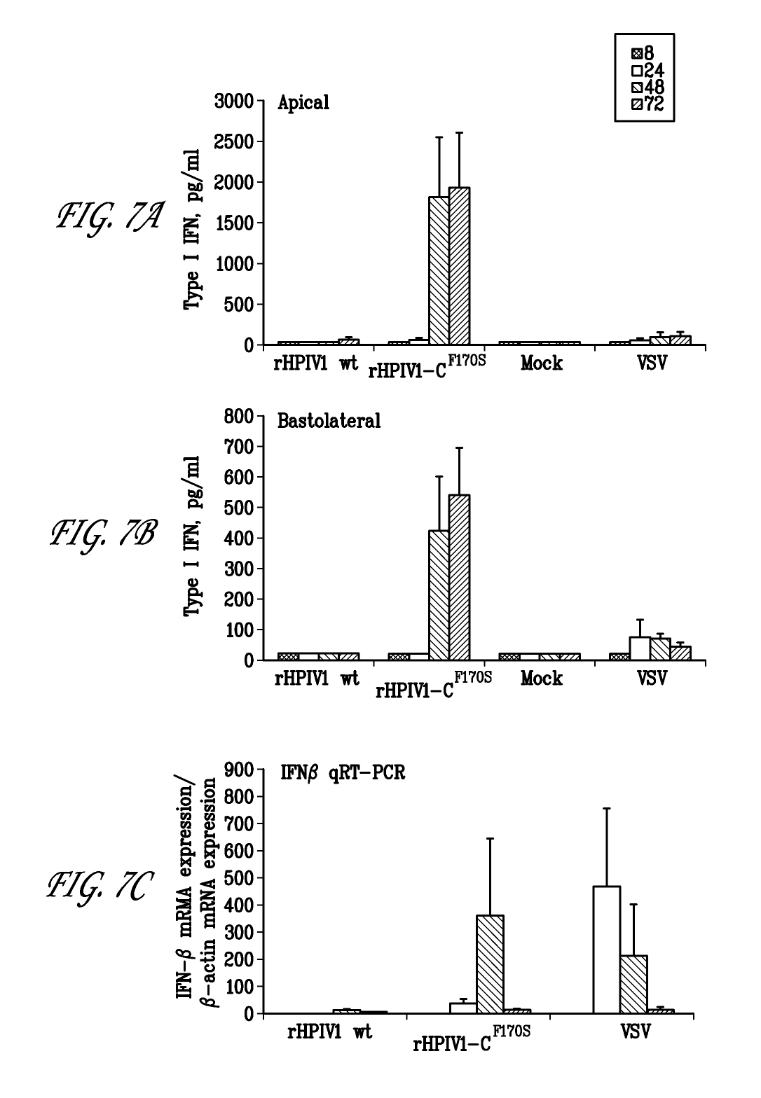

FIG. 5 shows comparisons of single cycle virus growth curves in HAE inoculated with rHPIV1 wt (A) or rHPIV1-C^{F170S} (B) at an MOI of 5.0 TCID₅₀/cell or with VSV (C) at an MOI of 4.2 PFU/cell, at 37° C. Virus titers were determined in the apical and basolateral compartments at 8, 24, 48 and 72 h p.i. Virus titers shown are the means of cultures from two donors±S.E., and the limit of detection is 1.2 log₁₀TCID₅₀/ml. Here, it can be seen that rHPIV1 also replicated efficiently in a single step growth curve following apical inoculation at a high input MOI (5.0 TCID₅₀/cell), (FIG. 5A). These growth curves were performed in the absence of added trypsin (FIGS. 4 and 5). Since HPIV1 typically requires added trypsin for cleavage and infectivity when grown in cell lines, such as Vero cells, this suggests that a trypsin-like enzyme capable of cleaving HPIV1 F is provided by HAE cultures. Influenza virus, another virus requiring serine protease activity at the apical surface for multicycle repication, also spread efficiently in HAE models in the absence of exogenous trypsin (Matrosovich, M. N., T. Y. Matrosovich, T. Gray, N. A. Roberts, and H. D. Klenk. 2004. Human and avian influenza viruses target different cell types in cultures of human airway epithelium. Proc Natl Acad Sci USA 101:4620-4; Thompson, C. I., W. S. Barclay, M. C. Zambon, and R. J. Pickles. 2006. Infection of human airway epithelium by human and avian strains of influenza a virus. J Virol 80:8060-8.). Attempts to isolate the required proteases that are responsible for cleaving viral proteins present in such models have identified some of these proteases (Bottcher, E., T. Matrosovich, M. Beyerle, H. D. Klenk, W. Garten, and M. Matrosovich. 2006. Proteolytic activation of influenza viruses by serine proteases TMPRSS2 and HAT from human airway epithelium. J. Virol. 80:9896-9898), but there are most likely a large number of proteases present in the human airway epithelium. Viral titers during infection were determined in both the apical and basolateral compartments, representing virus shed into the airway lumen and the serosal side of the epithelium, respectively. In general, viruses causing disease limited to the respiratory tract release virus via the apical surface only, whereas viruses released from both the apical and basolateral surfaces are typical of viruses that are able to spread systemically and cause disease in other tissues. This was demonstrated here by comparing growth curves for rHPIV1 wt to VSV (FIGS. 5A and 5C). As might be expected for a virus that is strongly pneumotropic, rHPIV1 wt was only detected in apical washes but not in basolateral compartments. In contrast, VSV, which is capable of systemic infection, was released into both sites after basolateral inoculation (FIGS. 5A and C).

The ciliated cells of the human airway epithelium have been shown to be major targets for other respiratory viruses including influenza, SARS coronavirus and paramyxoviruses such as RSV and HPIV3 (Zhang, L., A. Bukreyev, C. I. Thompson, B. Watson, M. E. Peeples, P. L. Collins, and R. J. Pickles. 2005. Infection of ciliated cells by human parainfluenza virus type 3 in an in vitro model of human airway epithelium. J Virol 79:1113-24; Zhang, L., M. E. Peeples, R. C. Boucher, P. L. Collins, and R. J. Pickles. 2002. Respiratory syncytial virus infection of human airway epithelial cells is polarized, specific to ciliated cells, and without obvious cytopathology. J Virol 76:5654-66.) FIG. 6 shows HPIV1 infection of ciliated cells without overt cytotoxicity. HAE were inoculated with HPIV1 wt or rHPIV1-C^{F170S} at an MOI of 5.0 TCID₅₀/cell or were mock-infected, and cells were processed at 24 and 48 h p.i. for histological analysis in cross section by immunofluorescence (6A) or H&E staining (6B) at 40× magnification or stained en face (6C). For histological immunofluorescence (6A, 6C), antibodies to HPIV1 (green) and alpha acetylated tubulin (red) were used to detect virus antigen and ciliated cells, respectively. Scale bars represent 20 μm (6A, 6B) and 40□m (6C). HPIV1 wt infects ciliated cells in HAE cultures, as observed by immunostaining histological sections of HAE (FIG. 4A and FIG. 6A). HAE support HPIV1 wt infection, and the pattern of infection seems to mimic that observed for other paramyxoviruses such as RSV and HPIV3. Therefore, HAE are a good model for studying HPIV1 infection. This model is further used here to characterize innate host responses in human airway epithelial tissues to infection and to determine the attenuation phenotypes of potential HPIV1 vaccine candidates. Infection with rHPIV1 did not induce any gross changes in morphology or integrity of the epithelium or any other evidence of cytopathic effect within 48 h of inoculation in comparison to mock-treated cells (FIG. 6B).

### Induction of Type I IFN During Virus Infection in HAE

The HPIV1 wt C accessory proteins inhibit the type I IFN response during infection, and this function is eliminated by a point mutation, F170S, in the C ORF, which is present in all four species of C protein, C′, C, Y1, and Y2. In A549 cells, type I IFN was detected during infection with rHPIV1-C^{F170S} but not rHPIV1 wt. Therefore replication and IFN induction by rHPIV1-C^{F170S} and rHPIV1 wt in HAE were compared. As was observed for HPIV1 wt, rHPIV1-C^{F170S} targeted ciliated cells in HAE (FIG. 6A). In addition, rHPIV1-C^{F170S} grew at least as efficiently as HPIV1 wt in a high MOI single cycle growth curve (FIG. 5). However, although both viruses reached similar peak titers, the kinetics of replication were somewhat different. rHPIV1-C^{F170S} reached a peak in titer by 24 h p.i., at which point its titer was 100-fold higher than that of rHPIV1 wt. In contrast, rHPIV1 wt titers rose steadily until 72 h p.i. (FIG. 5). The differences in the kinetics of virus replication between the two viruses in HAE correlated with en face staining which demonstrated that a higher proportion of cells were positive for viral antigen following infection with rHPIV1-C^{F170S} compared to rHPIV1 wt at both 24 and 48 h p.i. (FIG. 6C). This finding was consistent for two independent donor sources of HAE (data not shown). To investigate the initial higher replication of rHPIV1-C^{F170S}, the mutant and wt virus stocks were re-titered on three different cell lines, LLC-MK2, A549 and Vero cells. This showed that there were no cell line-specific differences in titer or infectivity between the two viruses. Furthermore, the ratio of infectious virus to hemagglutination titer was similar for the three viruses, indicating that they were comparable in infectivity. Thus, the increased replication of the mutant virus did not appear to be due to a difference in the amount of input virus or its infectivity, but may reflect a difference in the level of gene expression.

Type I IFN could be readily detected following high MOI infection of HAE with rHPIV1-C^{F170S} but not rHPIV1 wt FIG. 7 shows a comparison of the type I IFN response in HAE inoculated with rHPIV1 wt and rHPIV1-C^{F170S}. HAE were inoculated with rHPIV1s (MOI=5.0 TCID₅₀/cell), VSV (MOI=4.2 PFU/cell) or were mock-infected, and type I IFN mRNA and secreted protein were quantitated at 8, 24, 48 and 72 h p.i. A type I IFN bioassay was used to quantitate secreted type I IFN in the apical (7A) and basolateral compartments (7B) compared to an IFN-β standard. Type I IFN concentrations are expressed in pg/ml±S.E., and are the means of duplicate cultures. The IFN-β standard has a specific activity of approximately 1 IU of antiviral activity per 5 pg. The limit of detection for type I IFN was 20.2 pg/ml. IFN-β mRNA expression was quantitated by qRT-PCR (7C). Total RNA was extracted from HAE at 8, 24, 48 and 72 h p.i., and IFN-β mRNA was measured by qRT-PCR using specific primers and Taqman probes that have been previously described (Spann, K. M., K. C. Tran, B. Chi, R. L. Rabin, and P. L. Collins. 2004. Suppression of the induction of alpha, beta, and lambda interferons by the NS1 and NS2 proteins of human respiratory syncytial virus in human epithelial cells and macrophages. J Virol 78:4363-9.). For each sample, the level of IFN-β mRNA was relative to that of β-actin and expressed as a fold increase compared to that for the mock-inoculated sample.

Specifically, type I IFN secretion was detected in the apical and basolateral compartments of rHPIV1-C^{F170S} infected HAE by 48 h p.i., as determined by a type I IFN bioassay (FIGS. 7A and B). In addition, significant IFN-β mRNA expression was detected as early as 24 h p.i. in this virus group, as determined by qRT-PCR (FIG. 7C), whereas IFN-α mRNA was not detected in any group (data not shown). VSV was used as a positive control for IFN induction, and a low level of type I IFN protein and IFN-β mRNA was detected by 24 h following VSV infection (FIG. 7). These results indicate that there is both lumenal and basolateral/systemic release of type I IFN after infection of HAE with rHPIV1-C^{F170S} or VSV. The expression of IFN-β but not IFN-α following infection with rHPIV1-C^{F170S} is consistent with previous findings in A549 cells, and implies a strong block of IFN-mediated signaling. The lack of expression of IFN-β by HAE cells following infection with rHPIV1 wt also is consistent with results with A549 cells but differs from previous results reported with MRC-5 cells. To investigate the role of the released type I IFN on the spread of HPIV1 in HAE, infection at a lower MOI (0.01 TCID₅₀/ml) in longer-term infections was initiated.

Evaluation of the Role of Type I IFN in Multi-Cycle Replication of rHPIV1-C^{F170S} in HAE

During a natural HPIV1 infection, infection is likely initiated at a low MOI via luminal inoculation of the airways. Therefore, in order to mimic natural infection, HPIV1 wt and mutant virus replication was evaluated using a multiple cycle growth curve in HAE at 37° C. at a low MOI of 0.01. FIG. 8 shows virus replication and type I IFN production during multi-cycle growth curves in HAE inoculated with rHPIV1 wt and rHPIV1-C^{F170S} at an MOI of 0.01 TCID₅₀/cell at 37° C. Virus titers (log₁₀ TCID₅₀/ml; line graph) and type I IFN concentrations (pg/ml; bar graph) were determined in apical washes on each day from day 0-7 p.i. The titers shown are means of duplicate donor cultures±S.E. The limit of detection for virus titers was 1.2 log₁₀TCID₅₀/ml and for type I IFN was 31.1 pg/ml. The area shaded in gray represents the overall difference in virus replication between rHPIV1 wt and rHPIV1-C^{F170S} after day 2 p.i.

Both rHPIV1 wt and rHPIV1-C^{F170S} grew efficiently in HAE, reaching peak titers of 8.5 and 8.1 log₁₀ TCID₅₀/ml, respectively. The kinetics of replication and extent of infection mirrored that observed in the high MOI growth curves (FIG. 5). The rHPIV1 wt reached a peak titer at day 4 p.i., which remained at a plateau of about 8.5 log₁₀ TCID₅₀/ml until day 7 p.i. (FIG. 8). In comparison, rHPIV1-C^{F170S} reached a peak titer of 8.1 log₁₀ TCID₅₀/ml much earlier, at day 2 p.i., and virus replication then dropped dramatically by day 4 p.i. to plateau at 5.6 log₁₀ TCID₅₀/ml (FIG. 8). In addition, determination of type I IFN concentrations in apical compartments by bioassay demonstrated no detectable type I IFN during HPIV1 wt infection, whereas, type I IFN was detected from days 2-4 p.i. in cells infected with rHPIV1-C^{F170S}. Interestingly, the decrease in virus titer during rHPIV1-C^{F170S} infection followed the detection of type I IFN secretion. We have shown that cells infected with rHPIV1-C^{F170S} are able to express type I IFN, and secreted IFN likely acts on neighboring cells to establish an antiviral state. Since HPIV1 is sensitive to an established type I IFN-induced antiviral state, these data suggest that type I IFN secretion from virus-infected cells protected neighboring cells from virus infection. This protection can be seen in the approximately 300-fold reduction of rHPIV1-C^{F170S} replication in comparison to rHPIV1 wt (as indicated by the shaded area in FIG. 8).

### Replication of HPIV1 Vaccine Candidates in HAE

Since the HAE culture model is a useful in vitro tool for evaluating HPIV1 replication in a setting that closely resembles in vivo replication in seronegative humans, this model can be used for pre-clinical evaluation of HPIV1 vaccine candidates. Two attenuated HPIV1 mutants, rHPIV1-C^{R84G/Δ170}HN^{T553A}L^{Y942A} and rHPIV1-C^{R84G/Δ170}HN^{T553A}L^{Δ1710-11} were studied in the HAE model. These viruses were chosen since they have previously been characterized in vivo (Table 3, 4 and 5) and are currently being considered as live attenuated virus vaccine candidates for HPIV1; clinical trials using rHPIV1-C^{R84G/Δ170}HN^{T553A}L^{Y942A} are currently in progress. These viruses were previously shown to possess a ts phenotype with in vitro shut-off temperatures (e.g., the lowest temperature at which there is a ≧100-fold reduction in replication compared to wt virus) of 38° C. and 35° C., respectively, and both mutants were attenuated for replication in AGMs (Bartlett, E. J., A. Castano, S. R. Surman, P. L. Collins, M. H. Skiadopoulos, and B. R. Murphy. 2007. Attenuation and efficacy of human parainfluenza virus type 1 (HPIV1) vaccine candidates containing stabilized mutations in the P/C and L genes. Virol J 4:67.). In the present experiments their replication in vitro in HAE was examined.

TABLE 5
Virus replication of HPIV1 wt and rHPIV1 mutants in African
green monkeys and human airway epithelial cells.

| | Lower respiratory tract of AGMs ^{a} | | Apical surface of HAE cells (37° C.) ^{b} | |
|---|---|---|---|---|
| | Mean peak | Reduction in | Virus titer, | Reduction in |
| | virus titer | replication vs | Day 5 (log₁₀ | replication vs |
| Virus | (log₁₀ TCID₅₀/ml) | HPIV1 wt (log₁₀) | TCID₅₀/ml) | HPIV1 wt (log₁₀) |
| 1 HPIV1 wt | 3.9 | — | 8.6 | — |
| 2 rHPIV1-C^{F170S} | 2.7 | 1.2 | 5.8 | 2.8 |
| 3 rHPIV1- | 0.6 | 3.3 | 4.3 | 4.3 |
| C^{R84G/Δ170}HN^{T553A}L^{Y942A} | | | | |
| 4 rHPIV1- | ≦0.5 | ≧3.4 | 2.5 | 6.1 |
| C^{R84G/Δ170}HN^{T553A}L^{Δ1710-11} | | | | |

^{a} Data has been previously published (Bartlett, E. J., A. Castano, S. R. Surman, P. L. Collins, M. H. Skiadopoulos, and B. R. Murphy. 2007. Attenuation and efficacy of human parainfluenza virus type 1 (HPIV1) vaccine candidates containing stabilized mutations in the P/C and L genes. Virol J 4: 67; Bossert, B., S. Marozin, and K. K. Conzelmann. 2003. Nonstructural proteins NS1 and NS2 of bovine respiratory syncytial virus block activation of interferon regulatory factor 3. J Virol 77: 8661-8.)
^{b} Virus titers were determined in low MOI growth curves in HAE cells (FIGS. 4 and 5).

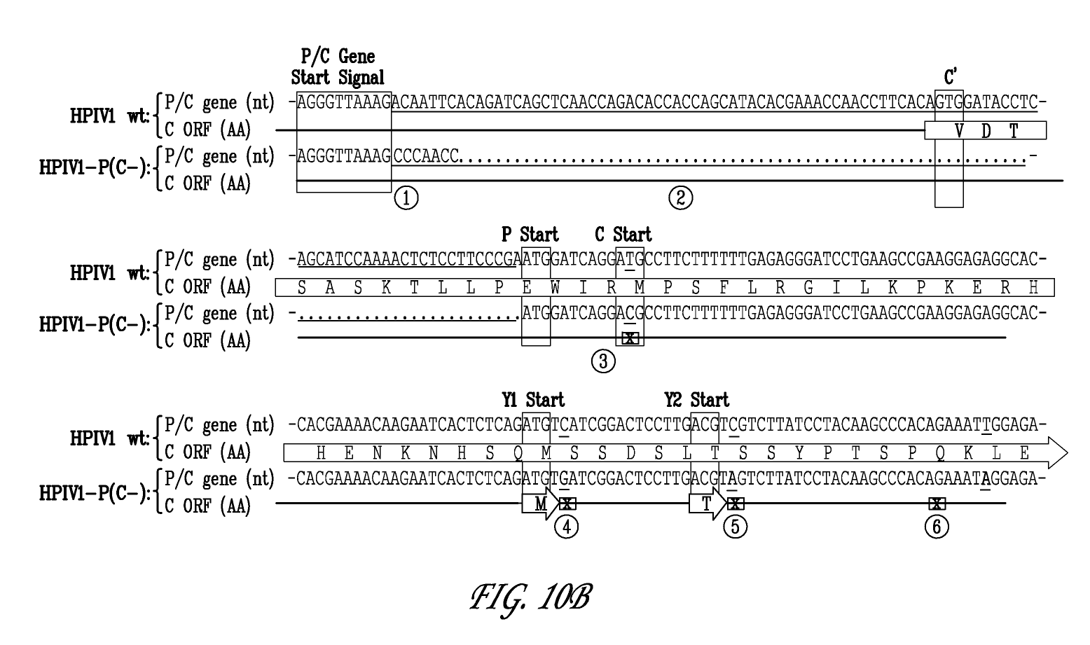

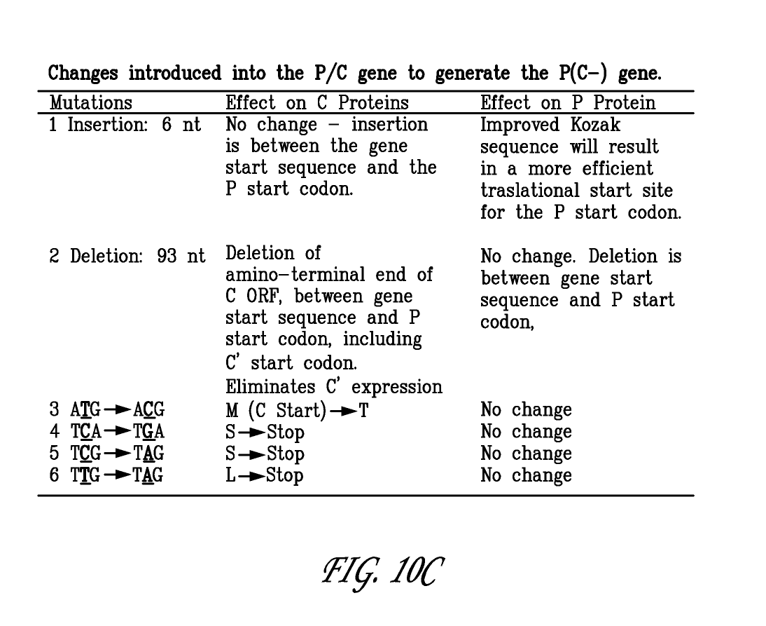

In order to simulate a natural virus infection, HAE were inoculated with the vaccine candidates at low MOI and replication was compared to rHPIV1 wt at 32° C. and 37° C., indicative of temperatures in the upper and lower respiratory tracts, respectively (FIG. 9). HAE were inoculated with rHPIV1 wt, rHPIV1-C^{R84G/Δ170}HN^{T553A}L^{Y942A} or rHPIV1-C^{R84G/Δ170}HN^{T553A}L^{Δ1710-11} at an MOI of 0.01 TCID₅₀/cell at 32° C. and 37° C. Virus titers (log₁₀ TCID₅₀/ml) were determined in apical and basolateral compartments each day from day 0-7 p.i. The virus titers shown are the means of triplicate donor cultures±S.E. for apical washes (samples from the basolateral compartments were negative for virus), and the limit of detection is 1.2 log₁₀TCID₅₀/ml. Viral titers determined in the apical washes over a seven-day period showed that rHPIV1 wt replicated efficiently at both temperatures, reaching peak titers of 8.5 and 9.1 log₁₀ TCID₅₀/ml, respectively by day 4 p.i. at 32° C. and 37° C. However both of the vaccine candidate viruses were severely restricted for replication in HAE and, unexpectedly, grew to higher titers at 37° C. than at 32° C. (FIGS. 9A and 9B). At 32° C., both viruses demonstrated little to no replication, even though this temperature is fully permissive for both viruses in monolayer cell lines, while at 37° C. there was low-level replication with a mean peak titer of 5.1 and 3.2 log₁₀ TCID₅₀/ml for rHPIV1-C^{R84G/Δ170}HN^{T553A}L^{Y942A} and the rHPIV1-C^{R84G/Δ170}HN^{T553A}L^{Δ1710-11} respectively (FIG. 9B). Thus, the rHPIV1-C^{R84G/Δ170}HN^{T553A}L^{Δ1710-11} virus demonstrated a higher degree of attenuation than rHPIV1-C^{R84G/Δ170}HN^{T553A}L^{Y942A}. Since both vaccine candidates were more attenuated than the virus containing only the C^{F170S} point mutation, which has previously been shown to share the same phenotype as C^{Δ170}, these data demonstrate that the HAE cells are also sensitive to the attenuation phenotype specified by other mutations, specifically, mutations in the L gene. Interestingly, there was no type I IFN detected in the apical or basolateral compartments of cultures infected with these viruses likely due to the low levels of virus replication.

### SUMMARY

HPIV1 wt readily infected HAE and replicated to high titer, yet failed to induce production of type I IFN. The virus was released exclusively at the apical surface of HAE, like many other respiratory viruses that do not cause viremia (or spread systemically). In contrast, VSV, which is not a common human pathogen but which is associated with systemic disease, albeit mild, in humans, was released at both the apical and basolateral surfaces. Ciliated cells of the human airway epithelium have been shown to be the target for many respiratory viruses including influenza virus, SARS coronavirus and paramyxoviruses such as RSV and HPIV3 (Zhang, L., A. Bukreyev, C. I. Thompson, B. Watson, M. E. Peeples, P. L. Collins, and R. J. Pickles. 2005. Infection of ciliated cells by human parainfluenza virus type 3 in an in vitro model of human airway epithelium. J Virol 79:1113-24; Zhang, L., M. E. Peeples, R. C. Boucher, P. L. Collins, and R. J. Pickles. 2002. Respiratory syncytial virus infection of human airway epithelial cells is polarized, specific to ciliated cells, and without obvious cytopathology. J Virol 76:5654-66.). Furthermore, ciliated cells have previously been identified as initiators of cytokine secretion during RSV infection (Mellow, T. E., P. C. Murphy, J. L. Carson, T. L. Noah, L. Zhang, and R. J. Pickles. 2004. The effect of respiratory synctial virus on chemokine release by differentiated airway epithelium. Exp Lung Res 30:43-57.). The results above show that HPIV1 can efficiently infect HAE cells, and that it specifically targets ciliated cells. One such HPIV1 mutant, rHPIV1-C^{F170S}, expresses defective C proteins and induces moderate to high levels of type I IFN, which in turn restricts replication of rHPIV1-C^{F170S} in HAE. Thus, the HPIV1 C proteins are critical regulators of the innate immune response in differentiated primary human epithelial cells in vitro.

In HAE, both rHPIV1 wt and rHPIV1-C^{F170S} replicated efficiently and reached similar mean peak titers. However, rHPIV1-C^{F170S} reached its peak titer by day 2 (at the onset of type I IFN production) compared to day 4 for rHPIV1 wt, which was true at both high and low MOI. Furthermore, immunostaining of HAE infected at high MOI also demonstrated a clear quantitative difference between the viruses at day 2 p.i. with many more cells staining positive in the rHPIV1-C^{F170S}-infected cultures compared to the rHPIV1 wt-infected cultures. Interestingly, a similar in vitro phenomenon has previously been observed in monolayer cultures with a SeV mutant containing the same mutation in the SeV C proteins. The F170S mutation in SeV had the effect of increasing gene transcription four-fold compared to its parent virus, with corresponding increases in RNA replication and virus replication (Garcin, D., M. Itoh, and D. Kolakofsky. 1997. A point mutation in the Sendai virus accessory C proteins attenuates virulence for mice, but not virus growth in cell culture. Virology 238:424-431.) This presumably accounts for the initial increased level of viral replication and viral antigen synthesis (detected by immunofluorescence) observed here for rHPIV1-C^{F170S} in HAE cells. Both HPIV1 and Sendai viruses containing the C^{F170S} mutation are attenuated in vivo (Bartlett, E. J., E. Amaro-Carambot, S. R. Surman, P. L. Collins, M. H. Skiadopoulos, and B. R. Murphy. 2006. Introducing point and deletion mutations into the P/C gene of human parainfluenza virus type 1 (HPIV1) by reverse genetics generates attenuated and efficacious vaccine candidates Vaccine. 24:2674; Garcin, D., M. Itoh, and D. Kolakofsky. 1997. A point mutation in the Sendai virus accessory C proteins attenuates virulence for mice, but not virus growth in cell culture. Virology 238:424-431).

The antiviral role of type I IFN became evident in HAE infected at low MOI. HPIV1 wt virus replicated to high titer and type I IFN was not induced. The HPIV1 wt virus titer persisted at a high level presumably due to the absence of IFN production in the cultures throughout the duration of the study. In contrast, rHPIV1-C^{F170S} replicated efficiently until IFN was detected and then titers decreased by a factor of 100 to 1000. Interestingly, rHPIV1-C^{F170S} induced the expression of IFN-β mRNA while the expression of IFN-α species was below the level of detection.

The 100 to 1000 fold difference in replication of rHPIV1-C^{F170S} and HPIV1 wt in HAE was similar to that observed in the upper and lower respiratory tract of AGMs (Table 5). It is evident that IFN production was associated with a reduction in virus replication; however, it was not sufficient to completely inhibit virus growth. Mutations of the C proteins are included in current HPIV1 vaccine candidates (Bartlett, E. J., A. Castano, S. R. Surman, P. L. Collins, M. H. Skiadopoulos, and B. R. Murphy. 2007. Attenuation and efficacy of human parainfluenza virus type 1 (HPIV1) vaccine candidates containing stabilized mutations in the P/C and L genes. Virol J 4:67.), HPIV1-C^{R84G/Δ170}HN^{T553A}L^{Y942A} and HPIV1-C^{R84G/Δ170}HN^{T553A}L^{Δ1710-11}, as detailed in the present invention.

Two live attenuated vaccine candidates for HPIV1, namely, rHPIV1-C^{R84G/Δ170}HN^{T553A}L^{Y942A} and the rHPIV1-C^{R84G/Δ170}HN^{T553A}L^{Δ1710-11} each contain mutations in C that specify the same IFN phenotype and attenuation phenotype as the C^{F170S} point mutation. In addition, both vaccine viruses contain a ts attenuating mutation in the L polymerase gene that restricts replication at 37° C. Both vaccine candidates replicate efficiently at 32° C. in Vero cells, the substrate for vaccine manufacture. We had anticipated that each vaccine candidate would replicate efficiently at 32° C. in HAE but would be restricted in replication at 37° C. due to the presence of the ts mutation. Surprisingly, the viruses were completely attenuated for replication in HAE at 32° C. following inoculation at low MOI and grew to very low levels at 37° C. The C and L gene mutations may collaborate to restrict replication at 32° C. by a mechanism that is undefined but can be addressed in HAE using mutants in which the various attenuating mutations are segregated. However, it is important to note that there was an additive effect in the level of attenuation specified by the combination of attenuating mutations in the P/C and L genes. Both vaccine candidates appear to be safe for evaluation in humans based on their highly restricted replication in AGMs and HAE. rHPIV1-C^{R84G/Δ170}HN^{T553A}L^{Y942A} replicated to slightly higher titers at 37° C. than rHPIV1-C^{R84G/Δ170}HN^{T553A}L^{Δ1710-11}, but was still significantly attenuated compared to rHPIV1 wt. IFN was not detected in cells infected with these viruses, which is most likely due to their highly restricted growth resulting in poor induction of the innate immune response. Comparing data from this in vitro study with previous in vivo studies, we have shown that attenuation, i.e. restriction in replication in HAE, correlates with the reduction in mean peak titers in AGMs (Table 5).

### Example 3

### HPIV1 Lacking Detectable Expression of C Proteins from the P/C Gene

C proteins are expressed by members of the Respirovirus, Morbillivirus and Henipahvirus genera but not by viruses that belong to the Rubulavirus and Avulavirus genera. The paramyxovirus C proteins studied to date are non-essential accessory proteins that contribute significantly to virus replication and virulence in vivo. The C proteins of Sendai virus (SeV), a member of the Respirovirus genus and the closest homolog of HPIV1, are the most extensively characterized.

The C proteins of SeV have been shown to have multiple functions that include inhibition of host innate immunity through antagonism of interferon (IFN) induction and/or signaling, regulation of viral mRNA synthesis by binding to the L polymerase protein, participation in virion assembly and budding via an interaction with AIP1/Alix, a cellular protein involved in apoptosis and endosomal membrane trafficking, and regulation of apoptosis (see below). SeV mutants containing deletions of all four C proteins are viable, but are highly attenuated in vitro and in mice. To date, the HPIV1 C proteins have not been as extensively studied as those of SeV. However, the HPIV1 C proteins, like the SeV C proteins, play a role in evasion of host innate immunity through inhibition of type I IFN production and signaling (Bousse, T., R. L. Chambers, R. A. Scroggs, A. Portner, and T. Takimoto. 2006. Human parainfluenza virus type 1 but not Sendai virus replicates in human respiratory cells despite IFN treatment. Virus Res 121:23-32; Van Cleve, W., E. Amaro-Carambot, S. R. Surman, J. Bekisz, P. L. Collins, K. C. Zoon, B. R. Murphy, M. H. Skiadopoulos, and E. J. Bartlett. 2006. Attenuating mutations in the P/C gene of human parainfluenza virus type 1 (HPIV1) vaccine candidates abrogate the inhibition of both induction and signaling of type I interferon (IFN) by wild-type HPIV1. Virology 352:61-73.). Type I IFN was not detected during infection with HPIV1 wild type (wt) in A549 cells, a human epithelial lung carcinoma cell line, but was induced during infection with a recombinant HPIV1 (rHPIV1) mutant bearing a F170S amino acid substitution in C, designated rHPIV1-C^{F170S} (Van Cleve, W., E. Amaro-Carambot, S. R. Surman, J. Bekisz, P. L. Collins, K. C. Zoon, B. R. Murphy, M. H. Skiadopoulos, and E. J. Bartlett. 2006. Attenuating mutations in the P/C gene of human parainfluenza virus type 1 (HPIV1) vaccine candidates abrogate the inhibition of both induction and signaling of type I interferon (IFN) by wild-type HPIV1. Virology 352:61-73.). HPIV1 wt virus, but not the rHPIV1-C^{F170S} mutant virus, inhibited the antiviral state induced by type I IFN, most likely due to inhibition of STAT1 nuclear translocation in human lung cells.

Many viruses have evolved strategies to regulate host cell apoptotic responses to virus infection. Apoptosis, a process of programmed cell death mediated by the activation of a group of caspases, results in systematic cellular self-destruction in response to a variety of stimuli. There are two major apoptotic pathways, the extrinsic and intrinsic pathways, which converge at a step involving the activation of the effector caspase 3. The activation of effector caspases, nuclear condensation and fragmentation, and cell death are the final steps in the apoptosis pathway. Viral proteins that are able to modulate the host apoptotic response include pro-apoptotic viral proteins such as West Nile virus capsid protein and bunyavirus NSs proteins, and anti-apoptotic viral proteins such as RSV NS proteins, Bunyamwera virus NSs and Rift Valley fever NSm protein. However, these studies are complex and some viral proteins such as influenza A virus NS1 have been reported to have both pro- and anti-apoptotic functions. SeV C proteins have been implicated in the regulation of apoptosis but their role in this process remains incompletely defined. A role for HPIV1 proteins in apoptosis has not been investigated to date.

In the present study, a rHPIV1 mutant was generated in which the C protein ORF was modified using reverse genetics to preclude expression of any of the four C proteins while maintaining expression of a wt P protein. Furthermore, this construct was made using the “wild type” arrangement of the P and C genes in overlapping reading frames. This is in contrast to the previously described studies in which the P and C genes were separated before mutation of either the P or C gene. The present mutant, designated rHPIV1-P(C-), was evaluated for replication in vitro and in vivo. In contrast to HPIV1 wt (but similar to rHPIV1-C^{F170S}), rHPIV1-P(C-) was found to induce a robust IFN response. In addition, in contrast to HPIV1 wt (and also in contrast to rHPIV1-C^{F170S}), rHPIV1-P(C-) induced a potent apoptotic response. This latter finding is unexpected from the prior art, and provides an additional pathway of attenuation that is advantageous for vaccine uses.

Both phenotypes appeared to contribute to attenuation in African green monkeys (AGMs) and in cultures of human ciliated airway epithelium.

### Materials and Methods

### Cells and Viruses

LLC-MK2 cells (ATCC CCL 7.1) and HEp-2 cells (ATCC CCL 23) were maintained in Opti-MEM I (Gibco-Invitrogen, Inc. Grand Island, N.Y.) supplemented with 5% FBS and gentamicin sulfate (50 μg/ml). A549 cells (ATCC CCL-185) were maintained in F-12 nutrient mixture (HAM) (Gibco-Invitrogen, Inc.) supplemented with 10% FBS, gentamicin sulfate (50 μg/ml) and L-glutamine (4 mM). Vero cells (ATCC CCL-81) were maintained in MEM (Gibco-Invitrogen, Inc.) supplemented with 10% FBS, gentamicin sulfate (50 μg/ml) and L-glutamine (4 mM). BHK-T7 cells, which constitutively express T7 RNA polymerase (Buchholz, U. J., S. Finke, and K. K. Conzelmann. 1999. Generation of bovine respiratory syncytial virus (BRSV) from cDNA: BRSV NS2 is not essential for virus replication in tissue culture, and the human RSV leader region acts as a functional BRSV genome promoter. J Virol 73:251-9. 745), were kindly provided by Dr. Ursula Buchholz, NIAID, and were maintained in GMEM (Gibco-Invitrogen, Inc.) supplemented with 10% FBS, geneticin (1 mg/ml), MEM amino acids, and L-glutamine (2 mM). Human airway tracheobronchial epithelial (HAE) cells were isolated from airway specimens of patients without underlying lung disease provided by the National Disease Research Interchange (NDRI, Philadelphia, Pa.) or from excess tissue obtained during lung transplantation, provided by the UNC Cystic Fibrosis Center Tissue Culture Core under protocols approved by the University of North Carolina at Chapel Hill (UNC) Institutional Review Board. Growth and differentiation of these cells on semi-permeable Transwell inserts at the air-liquid interface generated ciliated human airway epithelium (HAE), as previously described (Pickles, R. J., D. McCarty, H. Matsui, P. J. Hart, S. H. Randell, and R. C. Boucher. 1998. Limited entry of adenovirus vectors into well-differentiated airway epithelium is responsible for inefficient gene transfer. Journal of virology 72:6014-23.). All infections were incubated at 32° C. except where indicated otherwise.

Biologically-derived wt HPIV1 Washington/20993/1964, the parent of rHPIV1, was isolated from a clinical sample in primary AGM kidney (AGMK) cells, passaged 2 more times in primary AGMK cells and once in LLC-MK2 cells. This preparation has a wt phenotype in AGMs, and will be referred to here as HPIV1 wt, but it was previously referred to as HPIV1_{LLC1}. The rHPIV1 wt referred to in this study also contains a mutation in the HN gene, HN^{T553A}, that has previously been shown not to have an effect on virus replication (Bartlett, E. J., E. Amaro-Carambot, S. R. Surman, P. L. Collins, B. R. Murphy, and M. H. Skiadopoulos. 2006. Introducing point and deletion mutations into the P/C gene of human parainfluenza virus type 1 (HPIV1) by reverse genetics generates attenuated and efficacious vaccine candidates. Vaccine 24:2674-84.) and is therefore considered the equivalent of wt virus. rHPIV1 wt was generated as previously described (Bartlett, E. J., E. Amaro-Carambot, S. R. Surman, J. T. Newman, P. L. Collins, B. R. Murphy, and M. H. Skiadopoulos. 2005. Human parainfluenza virus type I (HPIV1) vaccine candidates designed by reverse genetics are attenuated and efficacious in African green monkeys. Vaccine 23:4631-46; Newman, J. T., S. R. Surman, J. M. Riggs, C. T. Hansen, P. L. Collins, B. R. Murphy, and M. H. Skiadopoulos. 2002. Sequence analysis of the Washington/1964 strain of human parainfluenza virus type 1 (HPIV1) and recovery and characterization of wild-type recombinant HPIV1 produced by reverse genetics. Virus Genes 24:77-92; Van Cleve, W., E. Amaro-Carambot, S. R. Surman, J. Bekisz, P. L. Collins, K. C. Zoon, B. R. Murphy, M. H. Skiadopoulos, and E. J. Bartlett. 2006. Attenuating mutations in the P/C gene of human parainfluenza virus type 1 (HPIV1) vaccine candidates abrogate the inhibition of both induction and signaling of type I interferon (IFN) by wild-type HPIV1. Virology 352:61-73.). The rHPIV1 wt was used in all experiments, with the exception that the biological HPIV1 wt was used for the hamster challenge and in the AGM studies, as indicated. The generation and characterization of the rHPIV1-C^{F170S} mutant also was described previously (Bartlett, E. J., E. Amaro-Carambot, S. R. Surman, J. T. Newman, P. L. Collins, B. R. Murphy, and M. H. Skiadopoulos. 2005. Human parainfluenza virus type I (HPIV1) vaccine candidates designed by reverse genetics are attenuated and efficacious in African green monkeys. Vaccine 23:4631-46.); this virus contains a single nucleotide substitution in the P/C gene that creates a phenylalanine-to-serine substitution at amino acid 170 (numbered relative to the C′ protein) that affects all four C proteins and is silent in the P protein. Media used for propagation and infection of HPIV1 wt and rHPIV1 mutants in LLC-MK2 cells did not contain FBS but contained 1.2% TrypLE Select, a recombinant trypsin (Gibco-Invitrogen, Inc.), in order to cleave and activate the HPIV1 fusion (F) protein, as described previously (Newman, J. T., S. R. Surman, J. M. Riggs, C. T. Hansen, P. L. Collins, B. R. Murphy, and M. H. Skiadopoulos. 2002. Sequence analysis of the Washington/1964 strain of human parainfluenza virus type 1 (HPIV1) and recovery and characterization of wild-type recombinant HPIV1 produced by reverse genetics. Virus Genes 24:77-92.). Purified virus stocks were obtained by infecting LLC-MK2 cells, followed by centrifugation and banding of virus containing supernatant in a discontinuous 30/60% (w/v) sucrose gradient, steps designed to minimize contamination with cellular factors, especially IFN. Recombinant vesicular stomatitis virus expressing the green fluorescent protein (VSV-GFP) was originally obtained from John Hiscott (Stojdl, D. F., B. D. Lichty, B. R. tenOever, J. M. Paterson, A. T. Power, S. Knowles, R. Marius, J. Reynard, L. Poliquin, H. Atkins, E. G. Brown, R. K. Durbin, J. E. Durbin, J. Hiscott, and J. C. Bell. 2003. VSV strains with defects in their ability to shutdown innate immunity are potent systemic anti-cancer agents. Cancer Cell 4:263-75.). Stocks of VSV were propagated in Vero cells and sucrose purified as indicated above.

Virus titers in samples were determined by 10-fold serial dilution of virus in 96-well LLC-MK2 monolayer cultures, using two or four wells per dilution. After 7 days of incubation, infected cultures were detected by hemadsorption with guinea pig erythrocytes, as described previously (Skiadopoulos, M. H., T. Tao, S. R. Surman, P. L. Collins, and B. R. Murphy. 1999. Generation of a parainfluenza virus type 1 vaccine candidate by replacing the HN and F glycoproteins of the live-attenuated PIV3 cp45 vaccine virus with their PIV1 counterparts. Vaccine 18:503-10.). Virus titers are expressed as log₁₀ 50% tissue culture infectious dose per ml (log₁₀ TCID₅₀/ml). VSV stock titers were determined by plaque assay on Vero cells under a 0.8% methyl cellulose overlay.

### Antibodies

Polyclonal antisera directed against the HPIV1 C or P proteins were generated by repeated immunization of rabbits with the following KLH-conjugated peptides: (i) QMREDIRDQYLRMKTERW (SEQ ID NO: 3; amino acid (aa) residues 153-170 of HPIV1 C′; directed against the carboxyl terminal region of C′, C, Y1 and Y2), for Skia-31; and (ii) RDPEAEGEAPRKQES (SEQ ID NO: 4, aa 10-24 of P), for Skia-2. Antisera were generated at Spring Valley Labs (Woodbine, Md.). Two murine monoclonal antibodies directed against the HPIV1 HN protein, designated 8.2.2.A and 4.5, were kindly provided by Dr. Yasuhiko Ito (Komada, H., S. Kusagawa, C. Orvell, M. Tsurudome, M. Nishio, H. Bando, M. Kawano, H. Matsumura, E. Norrby, and Y. Ito. 1992. Antigenic diversity of human parainfluenza virus type 1 isolates and their immunological relationship with Sendai virus revealed by using monoclonal antibodies. J Gen Virol 73:875-84.).

Construction of Mutant rHPIV1-P(C-) cDNA

Nucleotide insertions, deletions and substitutions were introduced into the P/C gene of rHPIV1 wt (FIG. 1A) in order to silence the expression of the C′, C, Y1, and Y2 proteins without affecting the P protein (FIGS. 1B and C). The 93 nucleotides between the P/C gene start signal and the P start codon, including the C′ start codon, were deleted (FIGS. 1B and C, mutation 2) and replaced with a 6 nucleotide insertion to act as a “linker” (FIGS. 1B and C, mutation 1). The sequence immediately upstream of the P start codon was modified by the addition of the “linker”: CGA(ATG) to AAC(ATG), making the P start site more efficient by Kozak's rules and reducing translational initiation at the downstream start codons (FIGS. 1B and C, mutation 1). The C start codon was modified (ATG to ACG) (FIGS. 1B and C, mutation 3), and three codons were converted to stop codons, including one immediately downstream of the Y1 start codon (TCA to TGA), which will affect all of the C proteins except Y2, and two downstream of the Y2 start codon (TCG to TAG; TTG to TAG), which will affect all of the C proteins (FIGS. 1B and C, mutations 4, 5, and 6). All of the introduced changes are silent in the P protein. These changes were achieved using a modified PCR mutagenesis protocol described elsewhere (Moeller, K., I. Duffy, P. Duprex, B. Rima, R. Beschorner, S. Fauser, R. Meyermann, S, Niewiesk, V. ter Meulen, and J. Schneider-Schaulies. 2001. Recombinant measles viruses expressing altered hemagglutinin (H) genes: functional separation of mutations determining H antibody escape from neurovirulence. J Virol 75:7612-20.) and the Advantage-HF PCR Kit (Clontech Laboratories, Palo Alto, Calif.). The entire PCR amplified gene product was sequenced using a Perkin-Elmer ABI 3100 sequencer with the Big Dye sequencing kit (Perkin-Elmer Applied Biosystems, Warrington, UK) to confirm amplification of the desired sequence containing the introduced changes. Full-length antigenomic cDNA clones (FLCs) of HPIV1 containing the desired mutations were assembled in T7 polymerase-driven plasmids using standard molecular cloning techniques, and the region containing the introduced mutation in each FLC was sequenced as described above to confirm the presence of the introduced mutation and absence of adventitious changes. Each virus was designed to conform to the rule of six, i.e., the nucleotide length of each genome was designed to be an even multiple of six, a requirement for efficient replication of HPIV1.

Recovery of Infectious rHPIV1-P(C-)

rHPIV1-P(C-) was recovered using a reverse genetics system, similar to previously described methods (Newman, J. T., S. R. Surman, J. M. Riggs, C. T. Hansen, P. L. Collins, B. R. Murphy, and M. H. Skiadopoulos. 2002. Sequence analysis of the Washington/1964 strain of human parainfluenza virus type 1 (HPIV1) and recovery and characterization of wild-type recombinant HPIV1 produced by reverse genetics. Virus Genes 24:77-92.), in BHK-T7 cells constitutively expressing T7 polymerase (Buchholz, U. J., S. Finke, and K. K. Conzelmann. 1999. Generation of bovine respiratory syncytial virus (BRSV) from cDNA: BRSV NS2 is not essential for virus replication in tissue culture, and the human RSV leader region acts as a functional BRSV genome promoter. J Virol 73:251-9.) that were grown to 90 to 95% confluence in six-well plates. Cells were transfected with 5 μg of the FLC, 0.8 μg each of the N and P, and 0.1 μg of the L support plasmids in a volume of 100 μl of Opti-MEM per well. Transfection was carried out with Lipofectamine 2000 (Invitrogen, Inc., Carlsbad, Calif.), according to the manufacturer's directions. The transfection mixture was removed after a 24 h incubation period at 37° C. Cells were then washed and maintained in GMEM supplemented with amino acids and 1.2% TrypLE Select and transferred to 32° C. On day 2 following transfection, the supernatant was harvested. Virus was amplified by passage on LLC-MK2 cells and cloned biologically by two successive rounds of terminal dilution using LLC-MK2 monolayers on 96-well plates (Corning Costar Inc., Acton, Mass.). To confirm that rHPIV1-P(C-) contained the appropriate mutations and lacked adventitious mutations, viral RNA (vRNA) was isolated from infected cell supernatants using the QIAamp viral RNA mini kit (Qiagen Inc., Valencia, Calif.), reverse transcribed using the SuperScript First-Strand Synthesis System (Invitrogen, Inc., Carlsbad, Calif.), and amplified using the Advantage HF cDNA PCR Kit (Clontech Laboratories). The viral genome was sequenced in its entirety, confirming its sequence.

### Western Blot

LLC-MK2 monolayers grown in 6 well plates (Costar) were mock-infected or infected at an input multiplicity of infection (MOI) of 5 TCID₅₀/ml with sucrose-purified rHPIV1 wt or rHPIV1-P(C-). Cell lysates were harvested 48 h post infection (p.i.) with 200 μl of 1× Loading Dye Solution sample buffer (Qiagen, Inc) and purified on QIAshredder (Qiagen, Inc.) spin columns. Ten μl (for Skia-31 probing) or 6 μl (for Skia-2 probing) of each sample was reduced, denatured and loaded onto 10-well 10% Bis-Tris gels (Invitrogen, Inc.). Gels were run in MOPS buffer (Invitrogen, Inc.), and protein was transferred onto PVDF membranes (Invitrogen, Inc.) and blocked overnight at 4° C. in PBS/Tween (0.1%) containing 3% BSA. PDVF membranes were incubated with 15 ml of a 1:1000 dilution of primary antibody in PBS/Tween with 1% BSA at room temperature (RT) for 2 h and then were washed 3 times for 10 min with PBS/Tween. Membranes were incubated for 1 h at RT with a 1:20,000 dilution of peroxidase labeled goat anti-rabbit IgG (KPL, Gaithersburg, Md.) as the secondary antibody. After washing 3 times for 10 min with PBS/Tween, SuperSignal West Pico Chemiluminescent Substrate (Pierce, Rockford, Ill.) was added for 10 min at RT. Membranes were developed on Kodak MR films (Kodak, Rochester, N.Y.).

Kinetics of Replication of rHPIV1-P(C-)

The rHPIV1 wt and rHPIV1-P(C-) viruses were compared in multicycle growth curves. Confluent monolayer cultures of LLC-MK2 cells in 6-well plates were infected in triplicate at a MOI of 0.01 TCID₅₀/cell. Virus adsorption was performed for 1 h in media containing trypsin. The inoculum was then removed and cells were washed three times, after which fresh medium containing trypsin was added and then harvested as the day 0 sample and replaced with fresh media containing trypsin. On days 1-7 p.i., the entire supernatant was removed for virus quantitation and was replaced with fresh medium containing trypsin. Supernatants containing virus were frozen at −80° C., and virus titers (log₁₀ TCID₅₀/ml) were determined with endpoints identified by hemadsorption. Cytopathic effect (cpe) was visually monitored. The amount of cpe observed under the microscope was given a score ranging from 1-5 based on the percentage of cells in the monolayer showing cpe. cpe of less than 20% of cells was scored as 1; 21-40% as 2; 41%-60% as 3; 61-80% as 4; 81-100% as 5.

### Immunostaining and Confocal Microscopy

LLC-MK2 cells were seeded onto 24 well plates containing 12 mm glass cover slips, were mock-infected or infected with rHPIV1-P(C-) or rHPIV1 wt at a MOI of 10 TCID₅₀/cell, and were incubated for 72 h. Media was removed and cover slips were washed twice with PBS. Cells were then fixed with 3% formaldehyde solution in PBS for 40 min at RT, washed once with PBS, permeabilized with 0.1% Triton X-100 in PBS for 4 min at RT, and washed twice with PBS prior to blocking with PBS containing 0.25% BSA and 0.25% gelatin for 1 h at RT. HPIV1 HN staining was performed using a 1:4000 dilution of a mixture of HPIV1-HN 8.2.2.A and HPIV1-HN 4.5, two murine antibodies directed against the HPIV1 HN protein, kindly provided by Yasuhiko Ito, Mie University School of Medicine, as primary antibody. After incubation at RT for 1 h, cells were washed twice with PBS and stained with a 1:1000 dilution of Texas Red conjugated donkey anti-mouse IgG (Jackson Immunochemicals, West Grove, Pa.), as secondary antibody, for 1 h at RT. Activated caspase 3 was detected using a 1:25 dilution of a FITC-conjugated rabbit anti-human activated caspase 3 antibody (BD Pharmingen, San Jose, Calif.). Cells were washed twice with PBS and immediately mounted onto slides with the DAPI-containing antifade reagent, ProLong Gold (Invitrogen, Inc.). Slides were covered with foil and left to dry overnight at RT, then stored at −20° C. until microscopy was performed on a Leica SP5 confocal microscope.

### FACS Analysis

LLC-MK2, Vero, or A549 cells in 6-well plates were mock-infected or infected with rHPIV1 wt or rHPIV1-P(C-) at a MOI of 5 TCID₅₀/cell. Cells were harvested at 24, 48, and 72 h p.i. by scraping cells into 2 ml of fresh FACS buffer (PBS; 1% FBS) and pelleting at 1200 rpm for 10 min at 4° C. Cells were resuspended in 1 ml of 3% paraformaldehyde (PFA) and fixed for 15 min on ice, then rinsed twice in 3 ml FACS buffer. Cells were permeabilized and stained with the following antibodies diluted in FACS buffer containing 0.1% Triton-X-100: i) rabbit anti-human activated caspase 3 FITC (1:100; BD Pharmingen); and ii) mouse anti-PIV1 HN (1:2000 of a 1:1 mix of HPIV1-HN 8.2.2.A and HPIV1-HN 4.5). Staining was performed for 45 min at RT in a dark environment then cells were rinsed twice with 2 ml FACS buffer and stained with APC-conjugated goat anti-mouse IgG (1:1000; Jackson ImmunoResearch Laboratories, West Grove, Pa.), diluted in FACS buffer containing 0.1% Triton-X-100. Staining was carried out for 30 min at RT. Finally, cells were rinsed twice in FACS buffer and resuspended in 250 μl FACS buffer for analysis. Sample analysis was carried out on a FACSCalibur (BD Biosciences, San Jose Calif.) using CellQuestPro software. Further analysis was performed using FlowJo software (TreeStar Inc., Ashland, Oreg.).

### Type I IFN Bioassay

The amount of type I IFN produced by HPIV1-infected A549 cell cultures was determined by an IFN bioassay, as previously described (Van Cleve, W., E. Amaro-Carambot, S. R. Surman, J. Bekisz, P. L. Collins, K. C. Zoon, B. R. Murphy, M. H. Skiadopoulos, and E. J. Bartlett. 2006. Attenuating mutations in the P/C gene of human parainfluenza virus type 1 (HPIV1) vaccine candidates abrogate the inhibition of both induction and signaling of type I interferon (IFN) by wild-type HPIV1. Virology 352:61-73.). Type I IFN concentrations were determined by measuring the ability of samples to restrict replication of VSV-GFP on HEp-2 cell monolayers in the samples in comparison to a known concentration of a human IFN-β standard (AVONEX; Biogen, Inc., Cambridge, Mass.). Briefly, samples were treated at pH 2.0 to inactivate virus and acid-labile type II IFN prior to being serially diluted 10-fold in duplicate in 96-well plates of HEp-2 cells along with the IFN-β standard (5000 pg/ml). After 24 h, the cells were infected with VSV-GFP at 6.5×10⁴ PFU/well. After an additional 24 to 36 h, plates were read for GFP expression using a typhoon 8600 phosphorimager (Molecular Dynamics, Sunnyvale, Calif.). The dilution at which the level of GFP expression was approximately 50% of that in untreated cultures was determined as the end-point. The end-point of the AVONEX standard was compared to the end-point of the unknown samples, and IFN concentrations were determined and expressed as mean±SE (pg/ml).

### Evaluation of Replication of Viruses in Hamsters and Efficacy Against Challenge

Four to six week old Syrian golden hamsters in groups of 5 or 6 per virus were inoculated intranasally (i.n.) with 0.1 ml L-15 containing 10^{5.5} TCID₅₀ of rHPIV1 wt, rHPIV1-P(C-) or control (L-15 only) inoculum. On days 4 and 5 p.i, the nasal turbinates and lungs were collected as previously described (Newman, J. T., S. R. Surman, J. M. Riggs, C. T. Hansen, P. L. Collins, B. R. Murphy, and M. H. Skiadopoulos. 2002. Sequence analysis of the Washington/1964 strain of human parainfluenza virus type 1 (HPIV1) and recovery and characterization of wild-type recombinant HPIV1 produced by reverse genetics. Virus Genes 24:77-92.). Virus present in the tissue homogenates was quantified by titration on LLC-MK2 monolayers. Infected cells were detected on day 7 p.i. by hemadsorption with guinea pig erythrocytes. The mean titer (log₁₀ TCID₅₀/g tissue) was calculated for each group of hamsters. The limit of detection was 1.5 log₁₀ TCID₅₀/g. On day 28 p.i., hamsters that had been previously immunized were challenged i.n. with 10⁶ TCID₅₀ of HPIV1 wt in 0.1 ml in L-15. The nasal turbinates and lungs were collected for virus quantitation on day 4 p.i.

### Evaluation of Replication of Viruses in AGMs and Efficacy Against Challenge

AGMs in groups of two to four animals at a time were inoculated i.n. and intratracheally (i.t.) with 10⁶ TCID₅₀ of either HPIV1 wt or mutant rHPIV1 in a 1 ml inoculum at each site. Nasopharyngeal (NP) swab samples were collected daily from days 0 to 10 p.i., and tracheal lavage (TL) fluid samples were collected on days 2, 4, 6, 8 and 10 p.i. The specimens were flash frozen and stored at −80° C. until they were assayed in parallel. Virus present in the samples was titered in serial dilutions on LLC-MK2 cell monolayers in 96-well plates, and an undiluted 100 μl aliquot also was tested in 24-well plates. Following incubation for 7 days, virus was detected by hemadsorption, and the mean log₁₀TCID₅₀/ml titer was calculated for each sample day. The limit of detection was 0.5 log₁₀ TCID₅₀/ml. The mean peak titer for each group was calculated using the peak titer for each animal, irrespective of the day of sampling. On day 28 p.i., the AGMs were challenged i.n. and i.t. with 10⁶ TCID₅₀ of HPIV1 wt in 1 ml L-15 per site. NP swab and TL samples were collected for virus quantitation on days 2, 4, 6 and 8 post-challenge.

All animal studies were performed under protocols approved by the National Institute of Allergy and Infectious Disease (NIAID) Animal Care and Use Committee (ACUC).

### Viral Inoculation of HAE

Apical surfaces of HAE were rinsed with PBS to remove apical surface secretions and fresh media was supplied to the basolateral compartments prior to inoculation. The apical surfaces of HAE were inoculated with HPIV1s at a low input MOI (0.01 TCID₅₀/cell) in a 100 μl inoculum, and the cultures were incubated at 37° C. The inoculum was removed 2 h p.i., and apical surfaces rinsed for 5 min with PBS and then incubated at 37° C. At days 0-7 p.i., apical samples were collected by incubating the apical surface with 300 μl of media for 30 min at 37° C., after which the media was recovered. Samples were stored at −80° C. prior to determination of virus titer.

### Statistical Analysis

The Prism 5 (GraphPad Software Inc., San Diego, Calif.) one-way ANOVA test (Student-Newman-Keuls multiple comparison test) was used to assess statistically significant differences between data groups (P<0.05).

### Results

Construction and Recovery of a rHPIV1 Mutant not Expressing any of the Four C Proteins

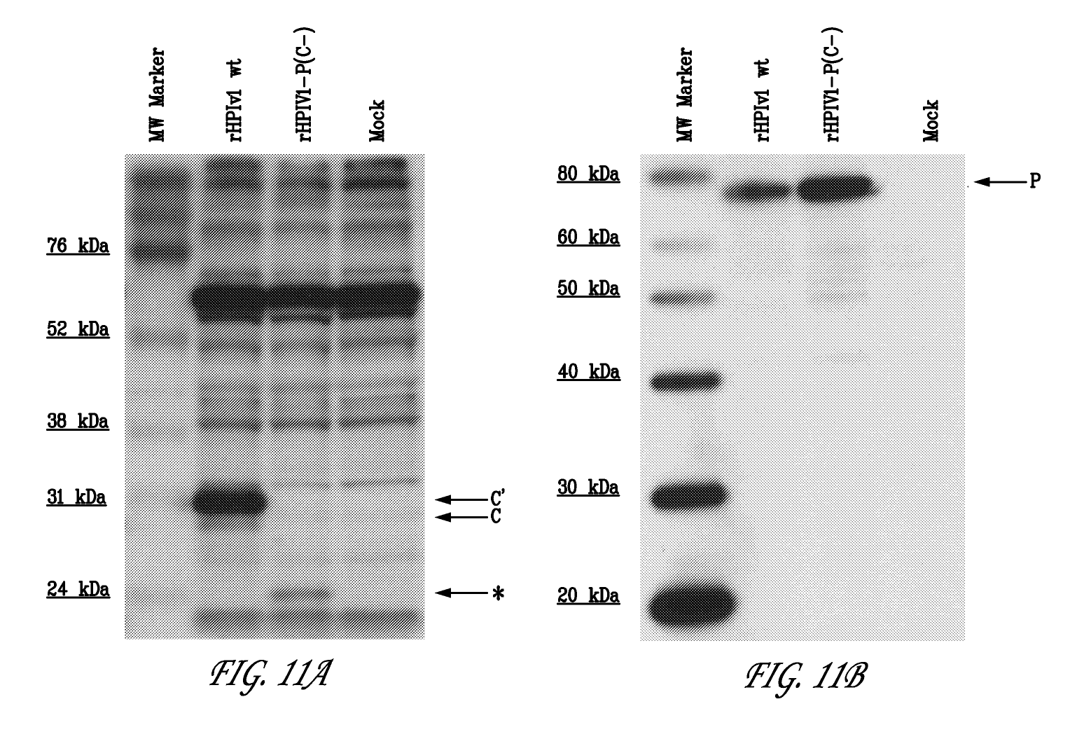

The P/C gene of HPIV1 wt encodes the phosphoprotein, P, in one ORF and four carboxy co-terminal C proteins, C′, C, Y1 and Y2, in a second, overlapping ORF (FIG. 10A). We engineered rHPIV1 to silence expression of all four C proteins without affecting the P protein, creating the mutant virus rHPIV1-P(C-) (FIGS. 10B and 10C). The changes introduced to silence expression of the C proteins included the deletion of the 3′ portion of the P/C gene containing the C′ start, conversion of the C start to an ACG codon, and the introduction of three stop codons into the C ORF immediately downstream of the Y1 and Y2 start codons (FIGS. 10B and 10C). Importantly, all of the introduced changes were silent in the P protein (FIGS. 10B and 10C). The Y1 and Y2 start codons were not modified since any changes introduced at these sites would have altered P protein amino acid assignments. The AUG to ACG change at the start site of C would not necessarily silence its expression entirely, since ACG functions (inefficiently) as a start codon for the C′ protein of SeV, but other changes at this site could not be accommodated without affecting P coding, and in any event any residual expression of C would be ablated by the three stop codons that were introduced downstream. The recombinant virus was recovered from this mutant cDNA in cell culture and the virus replicated to 8.0 log₁₀ TCID₅₀/ml. Sequence analysis of the entire virus genome revealed that rHPIV1-P(C-) contained all the intended mutations and no unintended changes.

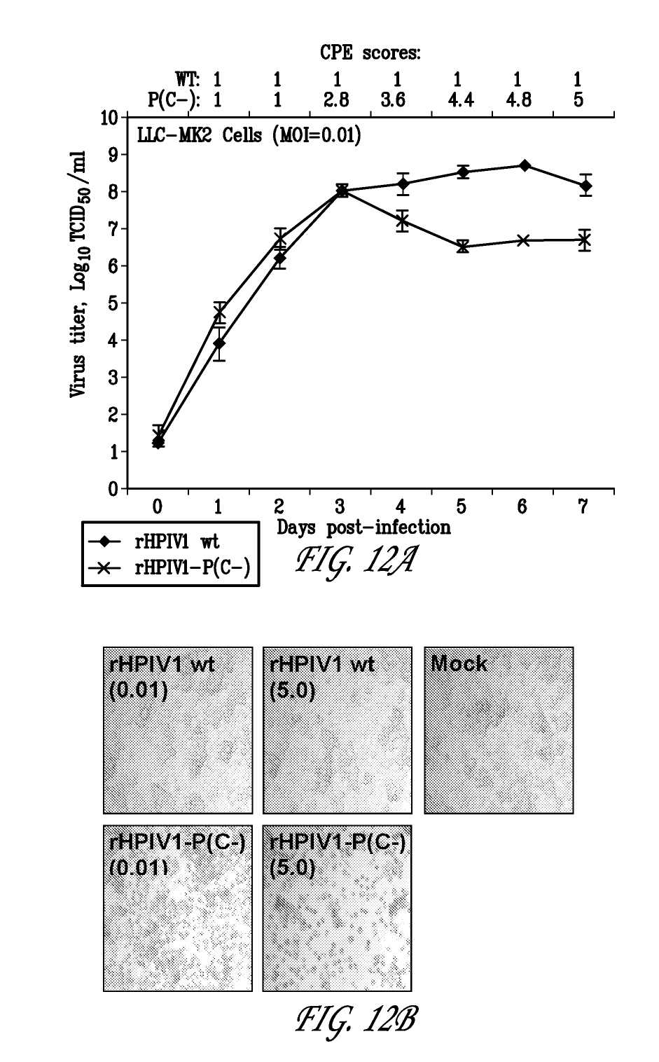

Western blot analysis of infected LLC-MK2 cell lysates using an antibody directed against the carboxy terminus of the C proteins demonstrated the expression of the C′ and C proteins in cells infected with HPIV1 wt, but not rHPIV1-P(C-) (FIG. 11A). C′ was found to be the most abundant C protein, and Y1 and Y2 were not detected. An additional unidentified species, indicated with an asterisk in FIG. 11A, was detected in cells infected with rHPIV1-P(C-), but not in cells infected with rHPIV1 wt (FIG. 11A). This species was not detected using an antibody directed against the amino terminus of the C′ and C proteins. An ATG codon in the C ORF that is downstream of the last inserted stop codon in Y2 could potentially give rise to a truncated protein that would be carboxy-coterminal with the C proteins and would be 157 aa in length, compared to 204 aa for the C protein and 175 aa for Y2 (FIG. 10A). This 47 aa difference in predicted size between C and the unknown protein in FIG. 2A would correspond to an approximately 5 kDa difference in the proteins' apparent molecular weights, consistent with the mobility difference observed in our Western blot (FIG. 11A). The P protein could be detected in both rHPIV1 wt- and rHPIV1-P(C-)-infected cells (FIG. 11B). The ability to recover the rHPIV1-P(C-) mutant indicates that the four wild type C proteins are not essential for replication in vitro, with the caveat that there was expression of a new species that may have been a truncated C protein.

The rHPIV1-P(C-) Mutant Replicates Efficiently In Vitro but Causes Increased cpe Compared to rHPIV wt

Multi-cycle replication of the rHPIV1-P(C-) mutant was assessed in LLC-MK2 cells infected at a MOI of 0.01 TCID₅₀/cell (FIG. 12A). rHPIV1-P(C-) and rHPIV1 wt replicated to similar titers until day 3 p.i., when rHPIV1 wt continued to increase in titer whereas rHPIV1-P(C-) decreased in titer. Concomitantly, rHPIV1-P(C-)-infected LLC-MK2 cells developed extensive cpe while rHPIV1 wt-infected cells did not (FIG. 12B). This also is evident in photomicrographs of LLC-MK2 cells taken 72 h following infection at MOIs of 0.01 or 5 TCID₅₀/cell (FIG. 12A). In LLC-MK2 cells infected at a MOI of 5 TCID₅₀/cell, increased cpe associated with rHPIV1-P(C-) but not rHPIV1 wt became evident at approximately 48 h p.i. Similarly, enhanced cpe associated with infection by the rHPIV1-P(C-) mutant was observed in A549 cells. In summary, rHPIV1-P(C-) and rHPIV1 wt replicated with equal efficiency early in infection, but there was a subsequent decrease in rHPIV1-P(C-) titers that was temporally associated with development of extensive cpe, a phenomenon not seen in rHPIV1 wt-infected cells.

Infection with rHPIV1-P(C-) Infection Induces Apoptosis

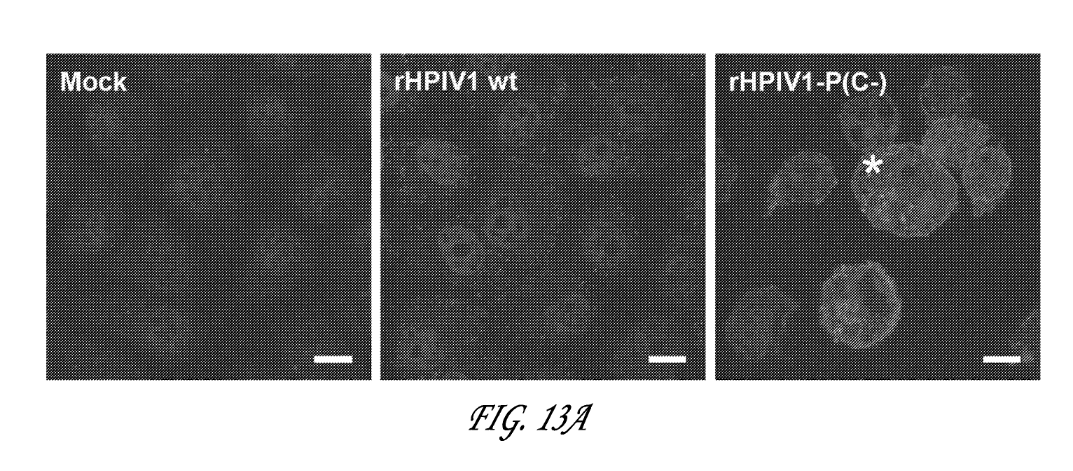

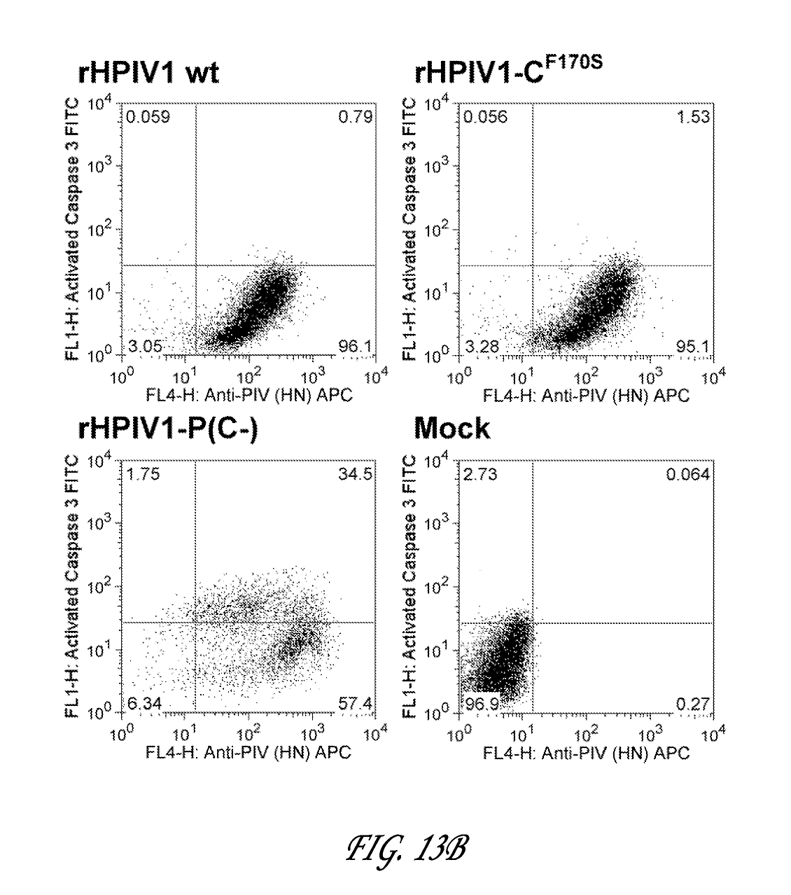

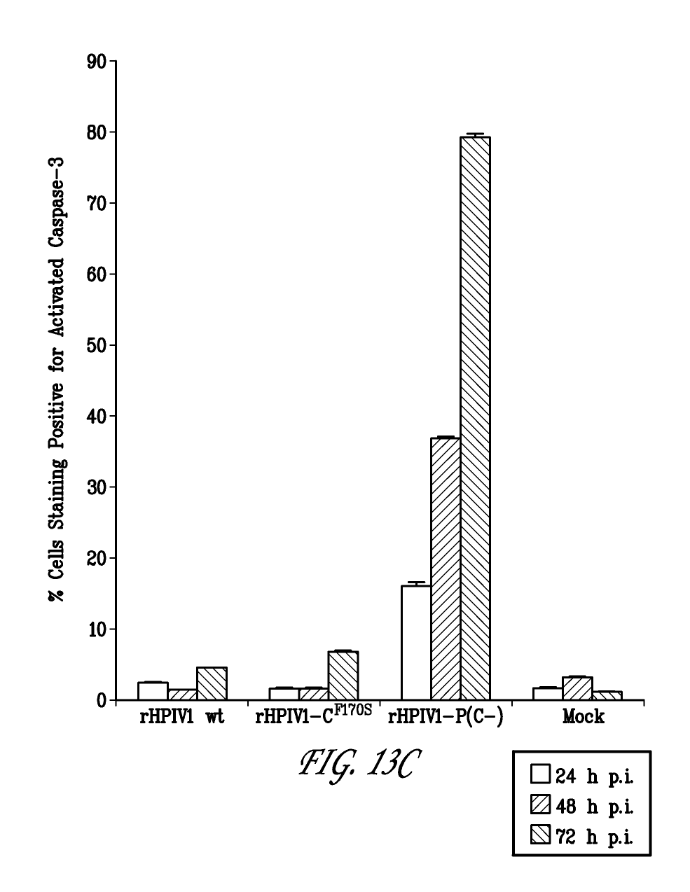

To further explore the basis of the enhanced cpe associated with the rHPIV1-P(C-) mutant, we assayed virus-infected LLC-MK2 cells for activation of caspase 3, the major effector caspase in the apoptotic pathway. Activated caspase 3 was visualized by immunofluorescence (FIG. 13A) and by FACS analysis (FIG. 13B) using an antibody that specifically recognizes the cleaved, activated form of the enzyme. Replicate LLC-MK2 monolayers were infected at a MOI of 10 TCID₅₀/cell, incubated for 24, 48 and 72 h, fixed, permeabilized, and stained with antibodies for HPIV1 HN (Texas-Red) and for activated caspase 3 (FITC). The HPIV1 HN antigen was detected in the vast majority of cells infected with either virus (FIG. 12A). By 72 h p.i., activated caspase 3 was detected in the majority of the rHPIV1-P(C-)-infected cells but not rHPIV1 wt-infected cells (FIG. 13A). In addition, cell rounding and nuclear condensation was seen in the majority of rHPIV1-P(C-)-infected cells but not in rHPIV1 wt-infected cells (FIG. 13A, asterisk), consistent with the interpretation that the rHPIV1-P(C-)-induced cpe is the direct result of virus-induced apoptosis.

We next determined the frequency of apoptosis in infected cells using flow cytometry. The rHPIV1-C^{F170S} mutant, which encodes a F170S substitution in C, and which has previously been associated with type I IFN induction and effective type I IFN signaling, but not with cpe in vitro, was included here for comparison. Replicate cultures of LLC-MK2 cells were infected with rHPIV1 wt, rHPIV1-C^{F170S} or rHPIV1-P(C-) at a MOI of 5 TCID₅₀/cell and, at 24, 48, and 72 h p.i., were fixed, permeabilized, and immunostained for HPIV1 HN protein and activated caspase 3 (FIG. 13B). More than 70% of cells in the rHPIV1-P(C-)-infected cultures were positive for activated caspase 3 by 72 h p.i., compared to approximately 5% and 7% in the rHPIV1 wt- and rHPIV1-C^{F170S}-infected cell cultures respectively (FIG. 13C). Similar studies in Vero and A549 cells confirmed that rHPIV1-P(C-) was a potent activator of caspase 3 activation while rHPIV1 wt was not, although the level of caspase 3 activation in these cells was lower than in LLC-MK2 cells. By 72 h p.i., approximately 12% and 18% of rHPIV1-P(C-)-infected Vero and A549 cell cultures, respectively, were positive for activated caspase 3, compared to approximately 4% of rHPIV1 wt-infected cell cultures for both cell types.

rHPIV1-P(C-), but not rHPIV1 wt, Induces Type I IFN Production and Signaling

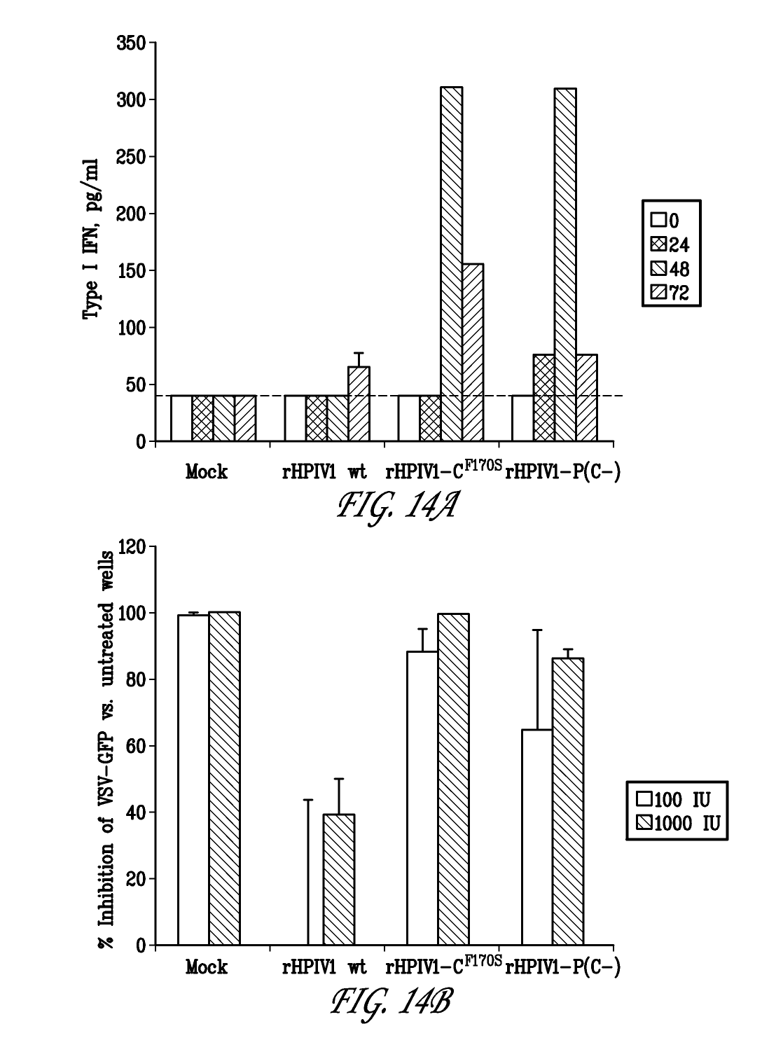

HPIV1 C proteins have been shown to inhibit production of and signaling by type I IFN. We have previously demonstrated that type I IFN was not detected during infection of A549 cells with HPIV1 wt but was efficiently produced in response to rHPIV1-C^{F170S}. To determine the relative effect of deleting all four C proteins on the ability of HPIV1 to inhibit the type I IFN response, we infected A549 cells at a MOI of 5 TCID₅₀/cell with rHPIV1-P(C-), rHPIV1 wt or rHPIV1-C^{F170S} and subsequently quantified type I IFN in medium supernatants using a bioassay based on the inhibition of infection and GFP expression by VSV-GFP (FIG. 14A). As shown previously, rHPIV1 wt inhibited the IFN response effectively (FIG. 14A), with barely detectable levels of IFN-β appearing late in infection, at 72 h p.i. In contrast, both rHPIV1-P(C-) and rHPIV1-C^{F170S} induced a robust type I IFN response, with IFN detectable in the supernatant as early as 24 h p.i. and until 72 h p.i., achieving a peak concentration of approximately 300 pg/ml at 48 h p.i. These data suggest that C proteins play a critical role in antagonism of type I IFN production but that IFN generation is unrelated to the apoptotic response seen with the C mutants since the F170S mutant generates similar levels of IFN with no observable cpe beyond wt HPIV1.

We have previously demonstrated that type I IFN signaling leading to the establishment of an antiviral state is inhibited following infection with HPIV1 wt, but not with mutants that encode defective C proteins, e.g. rHPIV1-C^{F170S}. To determine the relative effect of deleting all four C proteins on the ability of HPIV1 to inhibit type I IFN signaling, Vero cells were mock-infected or infected with rHPIV1 wt, rHPIV1-C^{F170S}, or rHPIV1-P(C-) at a MOI of 5 TCID₅₀/cell for 24 h, treated with 0, 100 or 1000 IU of IFN-β for 24 h, and infected with 200 PFU/well of VSV-GFP. The number of VSV-GFP foci were counted 48 h later and the percent inhibition due to IFN-β treatment was calculated relative to cells that did not receive IFN-β (FIG. 14B). In control cells that were not infected with rHPIV1, VSV-GFP replication was completely inhibited by IFN-β treatment. In contrast, infection with rHPIV1 wt ablated the ability of 100 IU/ml of IFN-β to inhibit VSV-GFP replication and blunted the inhibitory effect of 1000 IU/ml of IFN-β, indicating that rHPIV1 wt can prevent IFN-β signaling and the induction of an antiviral state. Infection with rHPIV1-P(C-) did not inhibit the antiviral effect of IFN-β at either concentration, an effect similar to that for rHPIV1-C^{F170S} (FIG. 14B). In summary, unlike rHPIV1 wt, rHPIV1-P(C-) is unable to inhibit both the production of type I IFN and the induction of an antiviral state by IFN-β.

rHPIV1-P(C-) is Highly Attenuated in Hamsters and Confers Protection Against wt HPIV1 Challenge

Golden Syrian hamsters were inoculated i.n. with 10^{5.5} TCID₅₀ of rHPIV1-P(C-) or rHPIV1 wt. Animals were sacrificed on days 4 and 5 p.i., and the level of virus replication in nasal turbinates and lungs was quantified by virus titration. In comparison to rHPIV1 wt, rHPIV1-P(C-) was restricted approximately 1000-fold in the upper respiratory tract (URT) and 250-fold in the lower respiratory tract (LRT) on both sampling days (Table 6). Although the replication of rHPIV1-P(C-) was highly restricted in hamsters, the animals were protected against i.n. challenge with 10⁶ TCID₅₀ of HPIV1 wt 28 days post-vaccination (Table 7). Replication of challenge virus in rHPIV1-P(C-)-immunized hamsters was restricted 200-fold in the URT and 16-fold in the LRT compared to non-immunized hamsters. Previous infection with rHPIV1 wt restricted replication of challenge virus even better than rHPIV1-P(C-), reducing challenge virus titers 1000-fold in the URT and 160-fold in the LRT (Table 7).

TABLE 6
Replication of rHPIV1-P(C-) in the upper
and lower respiratory tract of hamsters.

| | Mean virus titer (log₁₀ TCID₅₀/g) ± | | | |
|---|---|---|---|---|
| | SE on indicated day ^{b} | | | |
| Section 1.12 | Nasal turbinates | | Lungs | |
| Virus ^{a} | Day 4 | Day 5 | Day 4 | Day 5 |
| 1 rHPIV1 wt ^{c} | 5.2 ± 0.2 | 5.3 ± 0.4 | 4.6 ± 0.2 | 4.6 ± 0.4 |
| | (n = 10) | (n = 11) | (n = 10) | (n = 11) |
| 2 rHPIV1-P(C-) | 2.0 ± 0.2 ^{d} | 2.2 ± 0.2 ^{d} | 2.2 ± 0.2 ^{d} | 2.2 ± 0.1 ^{d} |
| | (n = 5) | (n = 5) | (n = 5) | (n = 5) |

^{a} Hamsters were inoculated i.n. with 10^{5.5} TCID₅₀ of the indicated virus.
^{b} The limit of detection was 1.5 log₁₀ TCID₅₀/g. The number of animals per group is indicated in parentheses.
^{c} The data for the rHPIV1 wt group represents two independent experiments.
^{d} Statistically significant reduction compared to rHPIV1 wt group at same time-point, P < 0.001 (Student-Newman-Keuls multiple comparison test).

TABLE 7
Protection against HPIV1 wt challenge in hamsters
following immunization with rHPIV1-P(C-).

| | |
|---|---|
| Immunizing | Mean HPIV1 challenge virus titer |
| virus or | (log₁₀ TCID₅₀/g) ± SE ^{b} |

| L-15 medium ^{a} | Nasal turbinates ^{c} | Lungs ^{c} |
|---|---|---|
| 1 rHPIV1 wt | ²1.5 ± 0.0 ^{d} | 1.7 ± 0.2 ^{d} |
| 2 rHPIV1-P(C-) | 2.2 ± 0.3 ^{d} | 2.7 ± 0.2 ^{d} |
| 3 Control | 4.5 ± 0.2 | 3.9 ± 0.4 |

^{a} Hamsters were inoculated i.n. with 10^{5.5} TCID₅₀ of the indicated virus and challenged on day 28 p.i. with 10⁶ TCID₅₀ of HPIV1 wt i.n., n = 5 for each group.
^{b} The limit of detection was 1.5 log₁₀ TCID₅₀/g.
^{c} Nasal turbinates and lungs from each group were harvested on day 4 post- challenge.
^{d} Statistically significant reduction compared to control group at same time-point, P < 0.01 (Student-Newman-Keuls multiple comparison test).

rHPIV1-P(C-) is Highly Attenuated in AGMs and Confers Protection Against HPIV1 wt Challenge

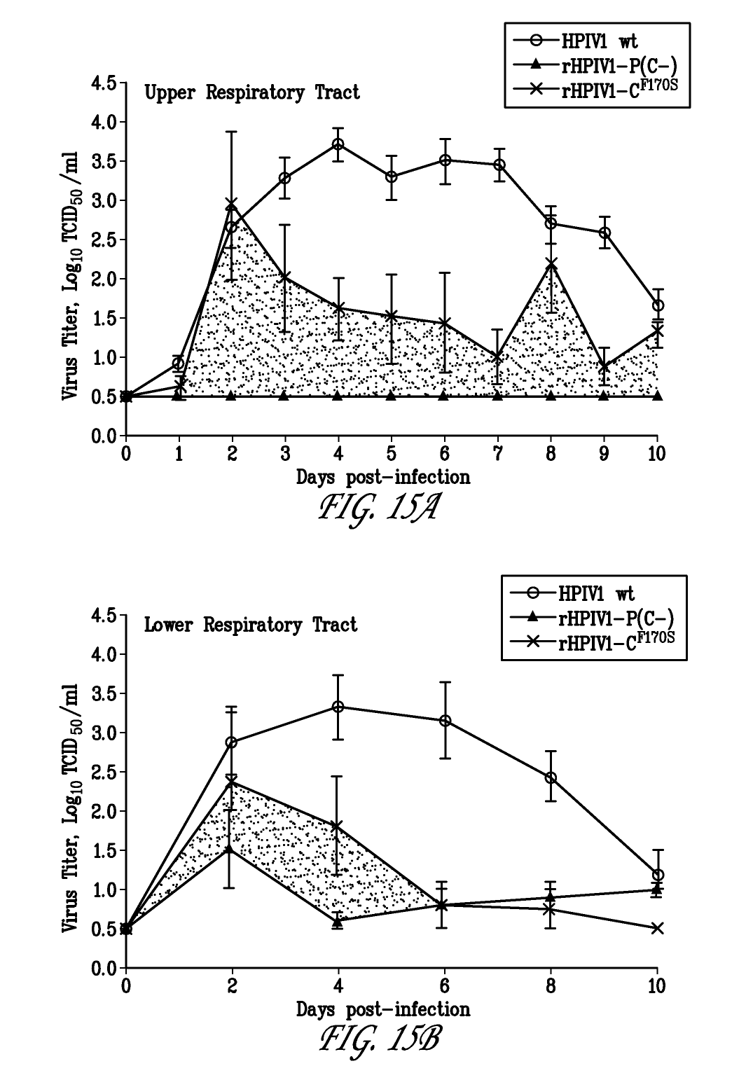

The attenuation phenotype of rHPIV1-P(C-) also was evaluated in AGMs. Following i.n. and i.t. inoculation of AGMs with 10⁶ TCID₅₀ of rHPIV1-P(C-) or HPIV1 wt at each site, virus titers were determined in NP swab samples (representative of the URT) on days 0-10 p.i. and TL samples (representative of the LRT) on days 2, 4, 6, 8 and 10 p.i. HPIV1 wt replication was robust in both the URT and the LRT of AGMs, with continued replication through day 10 p.i. (FIG. 15), and mean peak virus titers of 3.7 and 3.3 log₁₀ TCID₅₀/ml in the URT and LRT, respectively. In contrast, rHPIV1-P(C-) replication was undetectable in the URT and restricted but detectable in the LRT (FIG. 15). The level of replication of rHPIV1-P(C-) was compared with that of the previously described rHPIV1-C^{F170S} virus, a mutant that shares the IFN induction phenotype of rHPIV1-P(C-) but, as shown above, differs from rHPIV1-P(C-) in that it does not induce apoptosis. rHPIV1-P(C-) was found to be more attenuated than rHPIV1-C^{F170S}, and the difference in viral load between the two vaccination groups over time is indicated by the shaded area in FIG. 15. Despite its restricted replication, rHPIV1-P(C-) provided AGMs with protection against i.n. and i.t. challenge with 10⁶ TCID₅₀ HPIV1 wt per site at 28 days p.i. (Table 8). Replication of the HPIV1 wt challenge virus was restricted approximately 100-fold in the URT and the LRT of rHPIV1-P(C-)-immunized monkeys compared to non-immunized monkeys (Table 8).

TABLE 8
Protection against HPIV1 wt challenge in African green
monkeys following immunization with rHPIV1-P(C-).

| | Number | Mean peak titer (log₁₀ TCID₅₀/ml) ± SE ^{b} | |
|---|---|---|---|
| Immunizing | of | Nasopharyngeal | Tracheal lavage |
| virus ^{a} | animals | (NP) swab ^{c} | (TL) fluid ^{d} |
| 1 HPIV1 wt | 14 | 0.9 ± 0.2 ^{e} | 0.8 ± 0.1 ^{e} |
| 2 rHPIV1-P(C-) | 4 | 2.8 ± 0.5 ^{e} | 2.3 ± 0.4 ^{e} |
| 3 Mock-immunized | 14 | 4.7 ± 0.3 | 4.4 ± 0.4 |

^{a} Monkeys were inoculated i.n. and i.t. with 10⁶ TCID₅₀ of the indicated virus in a 1 ml inoculum at each site. On day 28 p.i., monkeys were challenged i.n. and i.t. with 10⁶ TCID₅₀ HPIV1 wt in a 1 ml inoculum at each site.
^{b} The limit of detection was 0.5 log₁₀ TCID₅₀/ml.
^{c} NP samples were collected on days 0, 2, 4, 6 and 8 post challenge. The titers on day 0 were ≦0.5 log₁₀ TCID₅₀/ml.
^{d} TL samples were collected on days 2, 4, 6 and 8 post challenge.
^{e} Statistically significant reduction compared to non-immunized group at same time-point, P < 0.001 (Student-Newman-Keuls multiple comparison test).

Replication of rHPIV1-P(C-) is Restricted in Human Airway Epithelial (HAE) Cells In Vitro

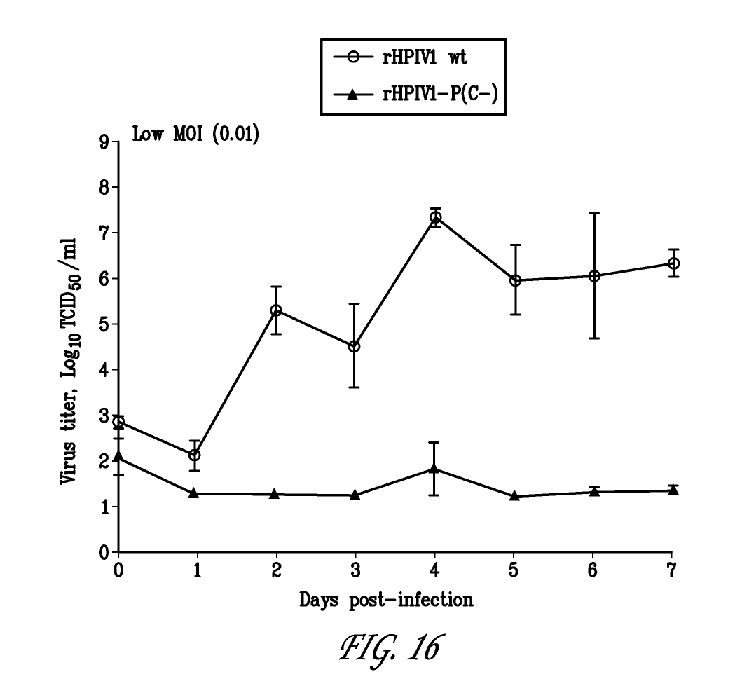

HPIV1 wt has been shown in Example 2 to infect ciliated apical cells in an in vitro model of the human airway epithelium. Here, we characterized the ability of the rHPIV1-P(C-) to infect HAE in a multiple cycle growth curve. Following apical inoculation at low input MOI (0.01 TCID₅₀/cell), rHPIV1 wt replicated efficiently in HAE, reaching a peak titer of 7.4 log₁₀ TCID₅₀/ml in the apical wash fluid. However, replication of the rHPIV1-P(C-) was severely restricted in human ciliated cells, reaching a barely detectable peak of 1.8 log₁₀ TCID₅₀/ml (FIG. 16).

### SUMMARY

A recombinant HPIV1 mutant, rHPIV1-P(C-), that does not express any of the four wild type C proteins but does express a wild type P protein was generated and characterized in vitro and in vivo. rHPIV1-P(C-) was found to replicate efficiently in vitro, implying that the HPIV1 C proteins are non-essential accessory proteins. However, rHPIV1-P(C-) expressed a novel protein not seen with rHPIV1 that may have been a truncated form of C, and this cautions against firmly concluding that the C-related proteins are completely dispensable. rHPIV1-P(C-) replicated with the same efficiency as HPIV1 wt early after infection of human- and monkey-derived cell lines, but its replication subsequently decreased coincident with the onset of extensive cpe that was not observed with rHPIV1 wt. The C proteins of SeV have been extensively characterized as non-essential gene products with multiple functions. However, SeV and HPIV1 differ with regard to the genetic organization of their accessory proteins and the phenotypes specified by accessory protein mutations. First, SeV encodes a V protein in addition to the C proteins, whereas HPIV1 does not. Second, deletion of all four C proteins in SeV significantly restricted its replication in vitro (Hasan, M. K., A. Kato, M. Muranaka, R. Yamaguchi, Y. Sakai, I. Hatano, M. Tashiro, and Y. Nagai. 2000. Versatility of the accessory C proteins of sendai virus: contribution to virus assembly as an additional role. J Virol 74:5619-28; Koyama, A. H., H. Irie, A. Kato, Y. Nagai, and A. Adachi. 2003. Virus multiplication and induction of apoptosis by Sendai virus: role of the C proteins. Microbes Infect 5:373-8; Kurotani, A., K. Kiyotani, A. Kato, T. Shioda, Y. Sakai, K. Mizumoto, T. Yoshida, and Y. Nagai. 1998. Sendai virus C proteins are categorically nonessential gene products but silencing their expression severely impairs viral replication and pathogenesis. Genes Cells 3:111-124.), whereas loss of the wild type forms of all four HPIV1 C proteins did not appear to reduce the replication efficiency of rHPIV1-P(C-) apart from the indirect effect of its enhanced cpe. Third, a six amino acid deletion in the N terminal region of the SeV C protein had a profound effect on replication in its natural host, i.e., rodents (Garcin, D., J. Curran, M. Itoh, and D. Kolakofsky. 2001. Longer and shorter forms of Sendai virus C proteins play different roles in modulating the cellular antiviral response. J Virol 75:6800-7.), whereas a similar mutation in HPIV1 C did not affect replication in non-human primates, the closest available animal model to its natural human host (Bartlett, E. J., E. Amaro-Carambot, S. R. Surman, P. L. Collins, B. R. Murphy, and M. H. Skiadopoulos. 2006. Introducing point and deletion mutations into the P/C gene of human parainfluenza virus type 1 (HPIV1) by reverse genetics generates attenuated and efficacious vaccine candidates. Vaccine 24:2674-84.). Fourth, the F170S mutation in SeV C induced apoptosis in primary mouse pulmonary epithelial cells (Itoh, M., H. Hotta, and M. Homma. 1998. Increased induction of apoptosis by a Sendai virus mutant is associated with attenuation of mouse pathogenicity. J Virol 72:2927-34.), whereas the same mutation in HPIV1 failed to specify this phenotype in the present study. Since the genetic organization of the accessory proteins of SeV and HPIV1 differ and since the phenotypes of C protein mutants differ significantly in vitro and in vivo, the functions of the HPIV1 C proteins cannot be reliably inferred from findings obtained with SeV C protein mutants, and therefore they must be determined directly. In the case of HPIV1, this information has added importance since live attenuated vaccine candidates that are presently being prepared for clinical trials include mutations in the C protein.

Replication of rHPIV1-P(C-) in cell culture peaked early and then decreased steadily coincident with the development of extensive cpe that was not observed with HPIV1 wt. This cpe was associated with caspase 3 activation, cell rounding, nuclear condensation and nuclear fragmentation, indicating that it was apoptotic in nature. Previously, the SeV mutant Ohita MVC11, which contains the C^{F170S} substitution, was observed to induce cell death in vitro while SeV wt did not (Itoh, M., H. Hotta, and M. Homma. 1998. Increased induction of apoptosis by a Sendai virus mutant is associated with attenuation of mouse pathogenicity. J Virol 72:2927-34.), indicating that the SeV C proteins also act as inhibitors of apoptosis. Similar to our observations of rHPIV1-P(C-), Ohita MVC11 titers peaked early and decreased concomitant with the induction of apoptosis. However, in contrast to Ohita MVC11, the HPIV1 mutant containing the homologous C^{F170S} substitution, rHPIV1-C^{F170S}, did not induce apoptosis. In addition, IRF-3 activation has been shown to be required for apoptosis during SeV infection in human cell lines (Peters, K., S. Chattopadhyay, and G. C. Sen. 2008. IRF-3 activation by sendai virus infection is required for cellular apoptosis and avoidance of persistence. J Virol 82:3500-8.), but we have shown that rHPIV1-C^{F170S} stimulates IRF-3 activation (Van Cleve, W., E. Amaro-Carambot, S. R. Surman, J. Bekisz, P. L. Collins, K. C. Zoon, B. R. Murphy, M. H. Skiadopoulos, and E. J. Bartlett. 2006. Attenuating mutations in the P/C gene of human parainfluenza virus type 1 (HPIV1) vaccine candidates abrogate the inhibition of both induction and signaling of type I interferon (IFN) by wild-type HPIV1. Virology 352:61-73.) but not apoptosis (present study). Taken together, this suggests that the mechanism of apoptosis induction and/or virus-mediated inhibition of apoptosis might differ between HPIV1 and SeV. SeV C deletion mutants have also been shown to induce apoptosis in vitro, whereas SeV wt does not (Itoh, M., H. Hotta, and M. Homma. 1998. Increased induction of apoptosis by a Sendai virus mutant is associated with attenuation of mouse pathogenicity. J Virol 72:2927-34; Koyama, A. H., H. Irie, A. Kato, Y. Nagai, and A. Adachi. 2003. Virus multiplication and induction of apoptosis by Sendai virus: role of the C proteins. Microbes Infect 5:373-8.), indicating that functional C proteins inhibit apoptosis that would otherwise be induced by SeV infection. In the case of HPIV1, the C proteins also inhibit apoptosis that otherwise is induced by HPIV1 infection. The absence of the four C proteins, and not the presence of the additional protein, that is associated with the apoptosis-inducing (and also the attenuation) phenotypes of rHPIV1-P(C-). Taken together, our data indicate that one function of the HPIV1 C proteins is to delay or prevent the apoptotic response of the infected cell.

The most extensively characterized function of paramyxovirus C proteins is their type I IFN antagonist activity. HPIV1 C proteins have been shown to disrupt the host type I IFN response by (i) inhibiting IRF-3 activation and thereby inhibiting the production of type I IFN, and (ii) inhibiting STAT nuclear translocation and thereby inhibiting the JAK-STAT signaling pathway. Our results support these previous findings by demonstrating that, in the absence of C proteins, type I IFN is produced in response to HPIV1 infection and is able to successfully establish an antiviral state in respiratory epithelial cells. In contrast, infection with HPIV1 wt inhibits type I IFN production as well as the establishment of an antiviral state that results from the activation of the JAK/STAT pathway (Goodbourn, S., L. Didcock, and R. E. Randall. 2000. Interferons: cell signalling, immune modulation, antiviral response and virus countermeasures. J Gen Virol 81:2341-64; Grandvaux, N., B. R. tenOever, M. J. Servant, and J. Hiscott. 2002. The interferon antiviral response: from viral invasion to evasion. Curr Opin Infect Dis 15:259-67; Levy, D. E., and A. Garcia-Sastre. 2001. The virus battles: IFN induction of the antiviral state and mechanisms of viral evasion. Cytokine Growth Factor Rev 12:143-56; Samuel, C. E. 2001. Antiviral actions of interferons. Clin Microbiol Rev 14:778-809; Taniguchi, T., and A. Takaoka. 2002. The interferon-alpha/beta system in antiviral responses: a multimodal machinery of gene regulation by the IRF family of transcription factors. Curr Opin Immunol 14:111-6; Weber, F., G. Kochs, and O. Haller. 2004. Inverse interference: how viruses fight the interferon system. Viral Immunol 17:498-515.). This pathway controls transcription of a group of more than 300 genes termed the IFN-stimulated genes, which have antiviral, antiproliferative, immunomodulatory and apoptosis modulating functions. Examples of other viral proteins having activity of interferon antagonists that play a role in apoptosis include the Bunyamwera virus NSs proteins (Kohl, A., R. F. Clayton, F. Weber, A. Bridgen, R. E. Randall, and R. M. Elliott. 2003; Bunyamwera virus nonstructural protein NSs counteracts interferon regulatory factor 3-mediated induction of early cell death. J Virol 77:7999-8008. Weber, F., A. Bridgen, J. K. Fazakerley, H. Streitenfeld, N. Kessler, R. E. Randall, and R. M. Elliott. 2002. Bunyamwera bunyavirus nonstructural protein NSs counteracts the induction of alpha/beta interferon. J Virol 76:7949-55.), the RSV NS1 and NS2 proteins (Bitko, V., O. Shulyayeva, B. Mazumder, A. Musiyenko, M. Ramaswamy, D. C. Look, and S. Barik. 2007. Nonstructural proteins of respiratory syncytial virus suppress premature apoptosis by an NF-kappaB-dependent, interferon-independent mechanism and facilitate virus growth. J Virol 81:1786-95; Spann, K. M., K. C. Tran, B. Chi, R. L. Rabin, and P. L. Collins. 2004. Suppression of the induction of alpha, beta, and lambda interferons by the NS1 and NS2 proteins of human respiratory syncytial virus in human epithelial cells and macrophages. J Virol 78:4363-9.) and the influenza A virus NS1 protein (Schultz-Cherry, S., N. Dybdahl-Sissoko, G. Neumann, Y. Kawaoka, and V. S. Hinshaw. 2001. Influenza virus ns1 protein induces apoptosis in cultured cells. J Virol 75:7875-81; Talon, J., C. M. Horvath, R. Polley, C. F. Basler, T. Muster, P. Palese, and A. Garcia-Sastre. 2000. Activation of interferon regulatory factor 3 is inhibited by the influenza A virus NS1 protein. J Virol 74:7989-96; Wang, X., M. Li, H. Zheng, T. Muster, P. Palese, A. A. Beg, and A. Garcia-Sastre. 2000. Influenza A virus NS1 protein prevents activation of NF-kappaB and induction of alpha/beta interferon. J Virol 74:11566-73; Zhirnov, O. P., T. E. Konakova, T. Wolff, and H. D. Klenk. 2002. NS1 protein of influenza A virus down-regulates apoptosis. J Virol 76:1617-25.). Our data demonstrate that the HPIV1 C proteins also act both as type I IFN antagonists and as apoptosis antagonists. Interestingly, however, the anti-IFN and anti-apoptosis activities of the HPIV1 C proteins were separable: while the rHPIV1-P(C-) and rHPIV1-C^{F170S} mutants were indistinguishable with regard to the induction of IFN production and signaling in cell culture, only rHPIV1-P(C-) induced apoptosis.

Since the type I IFN response and apoptosis are both components of the host's innate antiviral response, viruses that have lost the ability to inhibit these responses are often attenuated in vivo. The attenuation phenotype of the rHPIV1-P(C-) virus was evaluated in two in vivo models, i.e., in hamsters and AGMs, and an in vitro model of human ciliated airway epithelium (HAE). Replication of rHPIV1-P(C-) was restricted more than 1000-fold in the URT and 250-fold in the LRT of hamsters following i.n. inoculation. Despite this high degree of attenuation, it induced substantial protection against challenge with HPIV1 wt: challenge virus replication was restricted 200-fold in the URT and 15-fold in the LRT. The rHPIV1-P(C-) virus was also evaluated in AGMs to determine its potential for use as a live attenuated pediatric vaccine against HPIV1; AGMs are a more appropriate model since they are evolutionarily and anatomically closer to humans than are hamsters. In AGMs, replication of rHPIV1-P(C-) was not detectable in the URT and was barely detectable in the LRT. Despite this very high level of attenuation, significant protection against challenge was observed; challenge virus titers were reduced 79- and 125-fold in the URT and LRT, respectively. It is possible that rHPIV1-P(C-) will replicate more efficiently (and thus be more immunogenic and protective) in humans than in AGMs since it is a human virus that is more permissive in its natural host. For example, while HPIV1 causes significant respiratory disease in humans, infection of AGMs is asymptomatic. In order to obtain an independent assessment of attenuation prior to the initiation of clinical trials, replication of rHPIV1-P(C-) was characterized in a HAE model such as in Example 2, which uses primary human airway epithelial cells grown at an air-liquid interface to generate a differentiated, pseudo-stratified, ciliated epithelium that bears close structural and functional similarity to human airway epithelium in vivo. In our HPIV1 study, growth of rHPIV1-P(C-) in HAE cells was barely detectable, whereas rHPIV1 wt grew to high titer.

Previous studies of rHPIV1 C mutants demonstrated that in vivo attenuation correlated with the ability to stimulate an effective type I IFN response, including IFN production and IFN signaling (Bartlett, E. J., E. Amaro-Carambot, S. R. Surman, P. L. Collins, B. R. Murphy, and M. H. Skiadopoulos. 2006. Introducing point and deletion mutations into the P/C gene of human parainfluenza virus type 1 (HPIV1) by reverse genetics generates attenuated and efficacious vaccine candidates. Vaccine 24:2674-84; Van Cleve, W., E. Amaro-Carambot, S. R. Surman, J. Bekisz, P. L. Collins, K. C. Zoon, B. R. Murphy, M. H. Skiadopoulos, and E. J. Bartlett. 2006. Attenuating mutations in the P/C gene of human parainfluenza virus type 1 (HPIV1) vaccine candidates abrogate the inhibition of both induction and signaling of type I interferon (IFN) by wild-type HPIV1. Virology 352:61-73.). This was demonstrated in detail for rHPIV1-C^{F170S}. The rHPIV1-P(C-) virus was indistinguishable from rHPIV1-C^{F170S} with regard to the induction of type I IFN production and signaling to establish an antiviral state in vitro. However, rHPIV1-P(C-) was much more attenuated in AGMs than rHPIV1-C^{F170S}. This increased level of attenuation might be due to the induction of apoptosis by rHPIV1-P(C-), a property not shared by rHPIV1-C^{F170S}. We did not determine whether apoptosis was induced in vivo following rHPIV1-P(C-) infection, however, a SeV mutant containing the C^{F170S} mutation was shown to induce apoptosis both in vitro and in vivo, in the bronchial epithelium of mice, and this virus was also attenuated in its natural host (Garcin, D., M. Itoh, and D. Kolakofsky. 1997. A point mutation in the Sendai virus accessory C proteins attenuates virulence for mice, but not virus growth in cell culture. Virology 238:424-431; Itoh, M., H. Hotta, and M. Homma. 1998. Increased induction of apoptosis by a Sendai virus mutant is associated with attenuation of mouse pathogenicity. J Virol 72:2927-34.). This would suggest that the greater level of in vivo attenuation of rHPIV1-P(C-) versus rHPIV1-C^{F170S} is based on two combined effects: i) the ability (of both viruses) to activate the type I IFN response; and ii) the ability of rHPIV1-P(C-) (but not PIV1-C^{F170S}) to induce apoptosis. The shaded area in FIG. 15 highlights this added degree of attenuation that could be due to the induction of apoptosis. However, it is also possible that a C protein function other than inhibition of the IFN response and apoptosis contributed to the high level of attenuation of rHPIV1-P(C-) in AGMs.

In summary, we have demonstrated that the HPIV1 C proteins are non-essential viral proteins that play an important role in viral replication in vitro and in vivo and act as antagonists of the type I IFN response and of apoptosis. Our HPIV1 C protein deletion mutant is highly attenuated in the respiratory tract of non-human primates and in primary human airway epithelium yet this virus does confer significant protection against HPIV1 wt challenge in vivo. The HPIV1-P(C-) is much more attenuated that previously described point mutation or partial viruses having partial deletions in the C protein. Due to their highly attenuated phenotype, yet ability to confer protection against challenge by HPIV1, the rHPIV1-P(C-) or derivatives of this virus expressing N-terminal or C-terminal truncations of C proteins, are likely to be useful as vaccines for HPIV1.

1. An infectious, recombinant, self-replicating attenuated human parainfluenza virus type 1 (HPIV1) particle comprising a nucleocapsid protein (N), a nucleocapsid phosphoprotein (P), a large polymerase protein (L), a C protein and an FIN glycoprotein, and a partial or complete genome or antigenome encoding at least said N, P, C and L proteins, wherein
i) said C protein has a mutation in the codon encoding amino acid R84 such that another amino acid is encoded by said codon;
ii) said C protein has a deletion in the codon encoding amino acid 170;
iii) said FIN glycoprotein has mutation in the codon encoding amino acid T553 such that another amino acid is encoded by said codon;
iv) said L protein has a mutation in the codon encoding amino acid Y942 such that another amino acid is encoded by said codon or said L protein has a deletion of the codons encoding amino acids 1710 and 1711.

2. The infectious, recombinant, self-replicating attenuated HPIV1 of claim 1, in which the mutation i) in the C protein encodes glycine, the mutation iii) in the HN glycoprotein encodes alanine and the mutation iv) in the L protein encodes alanine.

3. The infectious, recombinant, self-replicating attenuated HPIV1 of claim 1, in which the mutation i) in the C protein encodes glycine, the mutation iii) in the HN glycoprotein encodes alanine and the mutation iv) in the L protein deletes the codons encoding amino acids 1710 and 1711.

4. The infectious, recombinant, self-replicating attenuated HPIV1 particle of claim 1 that is a complete virus.

5. The infectious, recombinant, self-replicating attenuated HPIV1 particle of claim 1 that is a partial viral particle.

6. The infectious, recombinant, self-replicating attenuated HPIV1 particle of claim 1, wherein the genome or antigenome further comprises a gene or genome segment of an antigenic determinant of a non-HPIV1 pathogen or a polynucleotide encoding a host cell immune regulatory protein.

7. The infectious, recombinant, self-replicating attenuated HPIV1 particle of claim 6, wherein the host cell immune regulatory molecule is selected from the group consisting of a cytokine, chemokine, enzyme, cytokine antagonist, chemokine antagonist, surface receptor, soluble receptor, adhesion molecule, or ligand.

8. The infectious, recombinant, self-replicating attenuated HPIV1 particle of claim 6 wherein the antigenic determinant is one or more determinants from a glycoprotein of a HPIV2, HPIV3, RSV, measles virus, influenza virus, or other non-HPIV1 pathogen.

9. A polynucleotide encoding the genome or antigenome of a HPIV1 according to claim 1.

10. An expression vector comprising:
i) a promoter which functions in a mammalian cell or in a cell free system operatively linked to
ii) a polynucleotide according to claim 9, that is operatively linked to
iii) a transcription terminator which functions in a mammalian cell or in a cell free system.

11. A recombinant cell comprising the expression vector of claim 10.

12. A method for producing an infectious, recombinant, self-replicating attenuated HPIV1 comprising expressing in a host cell a nucleocapsid protein (N), a nucleocapsid phosphoprotein (P) and a large polymerase protein (L) of a human parainfluenza virus, wherein said host cell further includes a polynucleotide according to claim 9, whereby an infectious viral particle comprising said N, P and L proteins and a partial or complete genome or antigenome encoding at least a nucleocapsid protein (N), a nucleocapsid phosphoprotein (P), a large polymerase protein (L), a C protein and a FIN glycoprotein is obtained, wherein said
i) said C protein has a mutation in the codon encoding amino acid R84 such that another amino acid is encoded by said codon;
ii) said C protein has a deletion in the codon encoding amino acid 170;
iii) said FIN glycoprotein has mutation in the codon encoding amino acid T553 such that another amino acid is encoded by said codon;
iv) said L protein has a mutation in the codon encoding amino acid Y942 such that another amino acid is encoded by said codon or said L protein has a deletion of the codons encoding amino acids 1710 and 1711.

13. The method of claim 12, in which said N, P and L proteins are expressed from more than one expression vector.

14. An immunogenic composition comprising the HPIV1 particle according to claim 1 and a pharmaceutically acceptable excipient or carrier.

15. The immunogenic composition of claim 14 that is formulated at a titer of 10³ to 10⁶ pfu/ml in the form of an aerosol or intranasal spray or droplet.

16. An infectious, recombinant, self-replicating attenuated human parainfluenza virus type 1 (HPIV1) particle comprising a nucleocapsid protein (N), a HPIV1 nucleocapsid phosphoprotein (P), a large polymerase protein (L), and a partial or complete genome or antigenome encoding at least said N, HPIV1 P, and L proteins, wherein said partial or complete genome or antigenome has a structure of overlapping open reading frames of HPIV1 P and HPIV1 C genes, and encodes a HPIV1 P protein but does not encode any HPIV C proteins.

17. A polynucleotide encoding the genome or antigenome of the HPIV1 of claim 16.


SEQUENCE LISTING (5 sequences)

SEQ ID NO: 1 - DNA, 15594 bp, Human parainfluenza virus
  misc_feature (1)..(15594): antigenome of HPIV1 C(R84G/del170)HN(T553A)L(Y942A) mutant virus
tggtctgttctcaaattctttatagctatataataattatgagaacagacaacattcaaaaagaatgatacaaaaatactcagtcaatttccctaaagaatagaacaattgagtagagaaatttgtttaatctattatttctactcttctatattctagtaacatacctcaggctacatagctataatcactatagtcgtctacacgagacagaacggggtttattaaactgacgagggttttaaaagtagttataaaaataaagaaatcagttcttggatatccagtgtgatacatattatagaagtttaaacagttactgttagaaactatattgggattctaaaaactaaagtgctctcctataactatgatatggattaacctcagaactaagtttaaattacagacctttactgggacaatgatcagaacatctgtatctgttacgataggttctataatgttcggaagatctttattgaaaattgaatggacttagagattctcctatatctagtataaatggacattcagaatcgaagaatagacgcagaaaataggctcaggtgagtttctaacatagagaattatgagatagtttacactattatgttaaaaaacacagatgatataaatcacatctattctttagagatgtattaaacaaatttaatccgagtttaggcttctaaacattacgaactaccattccagaatcgtatggactgctacgtgagggaaggaaattaagagattgataggttagtacttgaaaccgaaagagttgatcttaggtaaaaagatagaattatgataggagaaatctattaccttcttccaaacggttatgacataatgataggagatattatagtctaaatcctagaaaactatagtctatgtaaagacatctccgacccaatctacaaaagttgtgataattcgactgtagagaggtcatagaatttatgtttgaattaacaaaacaggtcagacatggattagaaccacgttaaaacgaatgattctattgcaggaataggggttgttccatacgttaggactagtgtgatattacaagtacattctggtggactagtgtaaacacaagaggtggaaggtacagtgttacttatttgggataacttaataagacattaagtaaggtctaatcacaaagtgtaagtagtaagggataggttcaactaggtcctaagggtaacttattttggaattgagaaactgtatttgagcactgtaataagttaaagaagggatggtcacggtgaagacttcctatttataaattaagagaaacaggcaactctagtgttcttaattgtggacttaatattatcaagtacgtcccaggattacatcgtagtattgttctgtcgtaacgtggacgtggaagaggctctatattagatagaaataggaataactgatctcctaagtcatttatccattcaaggttacgaaagtttgttgaccacgataattagggcttttcagagttaaccacactatttatagcgggtagttttccatctcaattaggatgtggactagaaggagaaagaagggaaaaatagagaagtattggagattcagcgtcttgaacactaaaactatgggaattgggggtcttccctcaagaaagcaagaagaaatttccatataacacaacttggagtcgtcaatataatagatcatcgtatgatagccgtagtcctagggtcaggagattctgaagaccatagggatatggagaatgagagttgtggaattaacttctaaaggactacattcaattgcaacttcccttatagaattattgattcggacagttaactgatatagaattatatcctagcagattttttcattcaagatctatgagaaagttactaagctcagcaagctctctacagtatctaaggttaaaaccagggagaaatcgatagagacggtttgatgtgttcatacgatctacagatcgatttttactttcaaaagagtaaagacgatataaacccagcaatagtgtttatttataaagttcgccatacgggagaacggttagtgcgtacttctccagttgtctcataagtgtatgactttaccgattgtcatagaacagaactaacctgttcaatccaggtatttgtccgagatgttgaggacgtagggtcttagcaaacttataaaatcctacactattacgtaacctgtcatgaaaatcctgacgtacactacacagaaattaatacgcatagtgtataggggtataaagtagggagggagactgcaaattgggcatttcacttatacgtttaactaactaatcttatgggggctttcaccttgttttattcccatatagttattggtatttaagccactaatttgataaatatagtagtagtaaatagtcacgatagtaaagaaattctaacagagatagattaactctgtaacacagacgttatcagtatcgacatgtatatgatcaacgagattaataaagtagtagtctggttattaagtatagttaacacacatgatgtagggattggaatgatttattgagatctagatacaggaactctcctagtcctagtatctaattgaacaataaaaggactcactagagattcaggttacatctagatcctccttataagttaagaactcctctaagatactatgtcgtaaataataagtttacattcacattatagtatccaaatctattactaggaacaaatatggagtataacttaagtttattcgagttaggccaattgtattaaacaactatttgatttaatcatagaaatctgagaggacggagaaaattacggtataatagtaatctataccattatttagctgagcgagcttgatttcatcgtgaacttttaaagtagacacatcgtcatagagagttagacaccctgtttaaacatcttcagctatgtccccactcattgaagtttaagagatttgaattcaatcgggaccaaactcgatattcacgacgaaggtaggtactataaagtagtcaaggtattcgggttcacatttggtaacgctaagaataacgacgaaaacggccgaacgaattcaagaattgtattggatcaactcggagactagaaagtagtcatcgtcttggttttattccctatgcataccgtcctgaaggtaacgattcacatagattcagctacaacagtccttctatcttggttcatatgtatcccaataagggaagactagatgttttaaatgtataaagactcggaagttatttactaggagaattattaagattgcccagacctcaaagatttggcacctaaccagaaggtattcaatctactttggtgtaaaaaacggagttaggataacggttaagatgacttgtgtatatgagtataagatataataggaattggccaaaagactcacaggaacaattccagagtataacattatctagtattaaatattcagaagaattctatgatatactattaggaggagaggactgtgatcgagaatgatctctaaagcaacatagttcgtagggccgatatcgaagagaatgtggacattcactcaatagattctagagcactcgatgagacccgttataccgaaaggataggtagtttttactacgattcaactctagaagaagtgatgaactaagacttttctctggactatcctctcctaatccactaaggacgttatgactagatcgacaatgtaaaaactaatatcaacaatatgaaacactcacaccctctaacgtacttattcccagactacgggttagattcttcgaactcagaggaccaagaactaagtagtgtgctatattatggacaaatagattgtctggacgaaactacttagagaaattcagacgatctcgacgttgacgacctagtggatataaagactgttttgtagatcgacagctgtagcgcaacttgggaggatacaatcgaccctagttataacgtgtagaattggttaaaaagggaaattttataactagaaactatcaactgcattatcatcctaattaccagtaaggattactatatgtatggacaaccgttcaaaatttttctcgttatgttatcggattctatccacttataggtaagagttatcgaaaccgatatctacaacgttacaaactcgtacgactggacaaaagtagatgttctcagagactggttttatgtgtagaacaatttcgaaactccgtgacaccctcctaaaagggtagtattatataggcaaacgacatatgtttgtaaaacgatgaatattaccagagtaaattaaaattaagtacaggttatagtttgtattgaagagagttccgtggctttatagaacactaaagaagtatctgcactcagaaaaaaacgaatatccaaacacactgtccttgagacctacagtgacgatatcgaactaacagaggaacatggtaacgactgtgagactgtggttgaaattgtcgacgttctacctaacgtgattatctctaatcacaggtattaaagaccgttattgggagataagggggagaacctaatacctatttctaaggccatagtagtactagaacctcaagaaacacgtaagatagtccatgacctgtcattcctagaggatgtatttaacatgaaaaaagatcttatcccacgtaggtcaatttctttcaaaatttgggcttatagagtaatgtagaaactggcttgttgcgacatgaaagtttagaggtcaactctgtcataaaaaagtttagccaccaattttttgtcgagtctcagagtatggggagccaactactcagacgtcgaaacttaagtaaaaatctaaagaaagaaagtagggttatgggtaaacttaacgtgaataagtataaaaactagttaaagagtacattataccctaataacatttgactcaacctagaaccttgtggactctgtctattacatcagtcagagaaatcatccagttaaagagggaaatggtagggtaaaagtgacttattgagtggatgaggaaaacgatcatcacaaagacgattatggacctgacgagagtagaatatacagtagaaccgtttctcagctgggagaacaaattaaagaaaaagaaactctgacattctatacaacttcgatagcagtaaattggtaagaggactgagatgtattaattattataggcgtcccaattttaataatagtaaatatttttggagctaattggatgctcagagaagactaagtccctgaaacattatatttaatgacagccctatatgacttagggtacgacgaaaggatccactatctcggaataggaagtatatttaacagtttagaagtagattaactccgagttatttaaaatttttaaatttaggatattttgatgatattaacagatgccgtgtaagtatgctttaacgtctcaacctaggaacacgtaagaactcaagttctgtatgtactaaccccttaagcgtaccgccggtaactggtggcacagaaagagatataggcaaataatattaacgtgtcttctaacgtactgtgagtatatcacagaaatcatactaaaaaaacctccttatgtatacacaggaatgaaatagccgtcgtcaataacgaagattcgatcctacaggtttgcaagattttttcctcttttaaagacgaaaaagtagttaactacagagagacttttagaaatagttactaaggtaatacgacagacgaagacttagacacatatataatagaaaataattttaacatacatcgagtcagttttgtacagagtactttcgaggaaaatttaatcaatgtcccagtaggttgacataatttcgttcactatttccaagatcatcactatattataatatatgtaggagtggttctaaccctttatttctcagctattcgagggtattaagaagggggaatgtacaataaaaactactggaaaatagctcagcaggtcgacttctgtataaggtagaaggaagctgttgtagtgttatgtaattatgattaagtccacattcctgtattggccaattaaaattaaataaataataatagtgatttagagatatatattagttttatatactaaatgtcagttcttttaacactcttaaactctgtagatttaccaaggggctgaataagaaaacgtaggtagagtatagtataaactacgatttggtacaattcttacccgctatggtacacaaaaataatataaatagtcaatatagaccctactatagatgtaggaaattactcagtttagggagaaagggaagttgtgtcggattaacagattcttataaattggtgttaagaactttaggtctactatcaagagaataattagatagtctctgacttttttacctagaataaaacagtgtataaagaccctataggtcagactcattgagattactcttttagcctattccaagttttcacattgctagttataggaattaaagaacaaattcctaaaacggatcgctagactaaaactagacggccccgctatttggaggtaaagactaaaacataaaccactataaataatatgacaatagaaaatttagtatcccaactaactatagatcgttttgcacttcaactcgttagaacgaaaaatgttagcctctcaagttcactgtaagacctatattctacagactccttaagactcaactgaggacaaataggtataaactgtttatccgtccgtaattgggattcaaaaagaataagataaacagtatatttacagataagtacgaaacaaaaatttaaaaatatgtagtatagaccaaaagtgtattaactatatcacaacccagaactagacgagttctacactaaaatgtataaaatccctatgaacagaacttgttgtatccaacattccataattccgaccgaaccaactaaagttgttacaccttcgtcatcgggaagggctttactcactatgtactacatcatcacatacgacggagatcaacatgtaagaactcagaatcgtacaactattaaagactccataaactcatgtaataccaccctaattgtgcactacacaaacgcatgtcacaccaacatcgttgtaactgacgtagtcctctatcacctatacgtagtcatatatgtggactatacgtaagagagcctgtagacaacatggtcaacgtcagaaccaaaggaccagctctgtcctgaagtactccgcgggtaaattaccagtaccccaacaactatagattactaggataaacgtccaacctcacggttggacttctagatcatatatacatctagaaaaatggatcaaattcatcggatggaagccgtggattcattaaaactcaataaccttaacagagctgttgttagaaaccggatagtctatttattaataactatgcttaattctgtaactgttgaacagaaaagaaatcggttcaatagaattctcgtagtaacgtttgtgagactaattgtaaccatgtagacaaatagtgtgtgaatcacagcggaacctcgcctcaacaattcggtggcatgggatccttttactcacataaaagttaaaaatagggtgaaggatgtgagcctatgtaacgtctttttcctaccagttttcaatatagaagtaaaaacatagctactctaaaccagaaagggaactctagattatacagtttatgatttagaagatatggaagtgagctcatcagacatcagagtaagtgtcacccgttcctcgtattgactattggggaacaaggacgtcgataatgtcttgtactaaaggacaacagcaactacagtatccatactctttaatggcccaaatttagtcctatgtatagacttaaattcctatacattggattaacattttggactatactgaagggatatagacgtgtaggaactcactaattcaaactacttatgcgtatataacgtagtggttaactattacttccatcagattgtgtaggactttaacaccacctaggtctttcatctgaaccaggtccattattactctataaccccaacaacgagtcatccccaaggggatgccctgtagaggtctttagcacacccaggtctcctctatacggcagacgtaccacttatcgttaccacaaaagcgtatagactcggttaaggacagacaacgtactgaagagataattaacacactctagagacgaaacgaacaaattgttataaccctagggcgaaacatgacttgaaacatacaaataccaggaactataatgaagaacagactaattaagacactaacagagaaactaataaaaccgtgaaagcaagtacctcctatgacagtactacaaacagaagtatgttcacagaacaaaatattaatccagttacgtatcctagtactattacttcctgttatgacatacgtaacaacaacgttagtcatcggtctacacacaggaaggacgaccacataattacacacacaaatggcaccttaacagtaaagcccaacatctggttatacttgataaacaaaagggaaaaagtcggtagcaaacatattagcctacgacaacggaatatccaactgacctaacagaaattgggattcaaaaagaatgaacaacatgtggactaaccacttcaacacctatctaaactaacaacatacatacgatgttacatgaactagactaaaaaattaacctcaacaatgggtacatccctaaagagtaagaactgaggtctcatattcgtaattgtccacttaatactcaacttaattagtaatgattatcagaagattgggacatatctatcatattatatggtgtatactaataatattcatacgtgtgttaataatagtactaatagactcatgagagacacaacacggtaggaggttgacgactatactaacgggaacggaagtactcgagacagaacctaaggagatcctttaaacaccgtctacgttctaaatttctttacagatgtccagattacctctgacgacctgggtgaaactactaaactaagggggttcatcacagagcaggggaaaataaccgtatatcaagataaggtaaatatggataatttggcgttaaccatatccagttcttacattgaggagaactcgctagaactaagtgaccataagacaaacagggcgtacatgtacacctacgttacgttaatcggtgatgtggtggtaactacttacgcttgaaaccatgatctaacaaataatgaaatcagtgtcccgtgaaactatgtaggggctcatatgtgaagacaaccaatagtccataattaacacatcctagagatcctgtatatatacggttaaaactaagatatgtcaaacactgtagacgtggaggattcctactacggaataactaatacattaaaccctaacgctgtacggtaagaagaggaagttataatattctatatctacttcgagacatatagttgtgcggaccataaagactattttaaccataaaattgattctcccattggtacataaaaaggtttagttgtagatagtgtcaagggaattggacaagtcacatttactacagtatatatagactaaataggaaaaatcaacatgaatcttaaagacaatataatcgtctcatctctctactattacggacgttccattctgaaaaaagaggatatcagggttctaacctcgacttacgacaacgattaagtctcatcacaacccactcgaactagggatcaaagttacggcatcagagtgtggaatcaagaggataacgtccagaatagagcagtaaatgttttagaacattacagaaattacgttaatagacaagaggatatggagaaacttactcaagatgtcttaacacacagaactgataccttagaaaataatcacgatacaggaaggaccgaagagcacgaagtcgattacgttacgggcgtcaataaactcgtcgacaccgatgaggatcacgataccatggttactgtcgtggtttcttagatcaaacacctaatagtaaacagtgtcaccacagtaatcaataacaatagtccctaaggacttctaggttccgtaggaagtctccttaatcttcagataaattatttaagaacataacttattaaactcaacaaggtgtaggtagaacatccagttaacttccatggttcgaattgtgatacatagatctaagtattctaggacgataaaattcattaaacgggagtaactaatattggggatgtaaactttcaaaaagatggccttaaactgtattactacttctattatcgttcgaactcatatgattcttctcctagagactaaaaacgtaaaaacagagatagaaccacaaataatacttaaatttaagacaaaaacaaatttaaaaacaagaacggaggcaaaaacagagaccggaaaaaagaaaaaaccaaaaacacatatgaaaacaaaaagaagggaaaaaacaagaacaaaaacaaaaacgaaaacaagaacaaacactagaacaggaaaagaaagaaacacaacaacaaaaagagagacgaatccgactcgaaaaactctgagaacagaaggaactacaaccatctcctgaaacagggatgcaaaaagaataaacttgactaacatagcttaactagacataaactaattaccatgctaaatctcgattacccacagtaataactcctcacatctgaatataggaggactaaaagggctacgaaaaacgttgttgtaaacccattatcgctttaagaaagccttaacgtccaaccttgtgtcgtagatgtgaacactaaagatgacttcgacgggtctactgatctaagtatacacctaaatgtaggtaatttcctatcgtttgacgaaaaaaggtacgttcaaccgagtaatctcaaaacctattacacggacaacgtacatgtacctttgaataaggtggatgttcgggctctctcttataatcgaagtagaagagttaaaataaaaacgtcataagttgacttatatagaagggttgaaaagaagaatactaaggatccacatggtacttcaactcgaagaaaagaggtagtagcagatcttgcccctcctaaggaaaaacaaggcaactatgaggacgggattcacattggtcgtctaagtgactataactcaagccattacgttctagacgttctcaaaaaccctggaatcgttaacattaacatggatcactacaaggtaactgtttatgttgggacttagactacagaaacagctgaccctctgtaactccacgatctcgatgaaataaccgtaatatatagtagggaacagagtcagccgacctggtaccatcatctcctcgtacctagcttagatggtacatatgatagtaaagaggtctagattgacaagaagcatgtcaataaaactccagttacgtccgaaaattattaagaactagacacggcattatgaaccgatacggttaaccattcctaggcctaggtgtctaacttattgaccaactaagacactctagtctatacgacggtctatctccaggaacgaatccacaaagtttcttagggttatcatcgtgtagttctatggactgaggtacaaaacctcctagaggatggaactgataagactacactccctaacgaaaaaacagtcctggacaaaactctccctctccaagatgacagggtaaaagaagcacactcttagaccccttggacatacaaagtcggtaaaggaaagaggcccgttttaaagaaactgggattcaaaaagaattaacaaaactccctcgaaaaactcaaccgagacctataactaaaactaacgaccaactttaacatctaagtacaaatcaatcaatcacttagatacagaaggagcttatcgaggtaatgtaaaaattggagaaccaaacaggctgtgaatatttccctaaactacattcggaagaagagatgagacgaatctcctgacaaaagctattgatcaaactcactcacgtctcacacaaaaagtcacctgccttagtccgcactacgtaacctacgaactccgagtaacaacaaaagaagatcatggcctaaaaactacagtagagagtcaagtaggagagaatagttcagtccgaacttgagaactggaggatctcgaaggtatctccccagtttggaacagaaaaactggaataacagaaagggaaaacacttctgactaccactggaacatcatagtgaccctaaagggcaaaagggcggagatagacattaatatacctcacacctatctaaccggtagtactcactcaagacaagaaaaaccataaggctcttggcaaacacttctagggacctacttaaataaaggagaacttaaacaaattcattacaagataaaagaagatgaaataagagttcactaaaaagttgactatattcgtatggtgtctctaatttacagtaaagaaacattaaacgtctaaaattgtacgcgaatcgtttatgtactgaacgtgaggatctattaagtttaagtacaacactaaagtaatgtggttctgaatgattattgaaccggtagagtagctgactacacaacgaccaaaggagaagtaaacgccaacgcagcacaagccctcaaccacctgaaaaacacaacaacaaacgtaacaaacaacacatagaacaaaaagacaccaacaccaacaagaaacctcaccgacgactgagaagcaagaaactgccaccttgaaaacccgaccaaaaccacaacaccagaagctccaacctcgataaaagtgaacaactaaacacctcaagacttgagacacaagagagactagaacaaaccaatgcgaactaccagcagccggcagaaaacaaggaaacatcttgccgtagaagttcgagacctgaacccaaccattactgtagtcaataagaacatgatacacacgacgatcaaaggtacgaagaaggtaataataaacaagatgggagaggtagtagtctattcccacatgaagaagcatggagaagaccgagttgaaccagaagaagggagaataatctcaagagtcggtagaaagacagaagtaggcattcatgactagagactacagaagagagtaaacctcttagaagcagctaagaccacaaagaaagtaacgggtataagagtccatctcccaaacctgatcaaggagcggatctccgggacacatatggaacaactagttcctaagatcgtacacagagacaaaaactgagtgaacaagaggatctctaaggtgaggggagtgaactagatactagaacccagctgcaactctaacgagacagaaacaacaatagcagccgaaacgtactaccgaacaaaagaaaacccaacagctactatggtacctcggtcgaagacagaagaggttaaagacacccgaacatcctattctgctgcagttcctcaggctactgtagactctcactaagaacaaaagcaccacggagaggaagccgaagtcctagggagagttttttcttccgtaggactaggtaagcccttcctctcaaaacctacgactccataggtgacacttccaaccaaagcacatacgaccaccacagaccaactcgactagacacttaacagtaagtgggattcaaaaagaatgaaaactagaccgaagataacaggttcataggacaatttaaggaggatagggacgtcggcgacatcgtagtagtagtaagagtatcagtcgtagtgacgaccacagagcaaataacaaacggaccgaatcagagacaaaagacagccggtcggaagcacgttagaacaaaagatatagtcactacagaagtagtaaacagagaagaagttcaagaagacgtggtgcttagcactaacacaccaacgactagctcactaagtaggtagaaagtaacgacagtaaacaggtcaagaacaacagggctagtcgtcacaggagctgaggtttacaatacagacatactagttaacggtgaagatatcgaggtggtggacatccgaacaccatgcgtggtaggggaggactttccaaacagtctaccacagaatcagagagaaaacgacatagacagtgtggatcaagaaggaggtctcgtgacgactaaaaccttagtcgtagaaatcgatgtcgaacaggttcatccttgtaaaggtacaggtttatacaagaaggtcaatgtatgacaacgtatcgaaacaaaacatggtgtcgttgcgggtaacgcatcgaggtgtcacgacctatcaaaggcccccgtttcagaggtacttgacccagagactcatatgtatattttccccgagcccgagggaaactatctatctatagttgttccgaagagttaaacaaatatagaccagagtctaaactatcacagtcccgccggtagaagcaaagatgtggtatgaactaccacaagtactttctacgattaggacgtagagaatacattaaaggatgttagacttacaagaagagattacatcaatttagactggacctacacaagtatcagtggtctcaaagatggtattctctatgattcgaaacaacactggagtagtgacgtggttatggaagttgacacagaggacacttctggtcccgtgaaaattgccaaggtaggacagacttacgaagattagataacttcttaggaaaagaatctggtcgtgatgaacaataccgaaattggttgtgataggtttgaacttgataatatcgaggctctgtacgtcctataggtatacagacttcgttacggagatctaggcgtaagagagaaacgggaaccttgttacccaagaacaactagtatccaggtttgttggtgagacaacaagaaagtatgaggtacagagaacaaaactgttacttgggtagacaaaaacaagaaaatcctagagagagatataacatatagtgtatgaactgtagtcgtaactgtggcaaacatcactctatattgaggcctgatgacatccggtaacgttcctcactatgatttttaggaggagaaactctcacaacaaatagacacaggttactcacccgattctttcaccaacgataattattaaatagacgtagtagacagtgtgagcccggattacacttctgtctttgccatgaagaaactggtccatattaacgaggaggcggtctgaataactacgagagtgaagaggaccttgatttacacagttttcatgaatcatcgggccggtaatatttttaaatatgattctagtttacagacactgaacagggaaatttcataatgaaattgggattttatgataaattataataatatataaggtttgttcaaaaaggagaacaaacca

SEQ ID NO: 2 - DNA, 15588 bp, Human parainfluenza virus
  misc_feature (1)..(15588): antigenome of HPIV1 C(R84G/del170)NH(T553A)L(del1710-1711) mutant virus
tggtctgttctcaaattctttatagctatataataattatgagaacagacaacattcaaaaagaatgatacaaaaatactcagtcaatttccctaaagaatagaacaattgagtagagaaatttgtttaatctattatttctactcttctatattctagtaacatacctcaggctacatagctataatcactatagtcgtctacacgagacagaacggggtttattaaactgacgagggttttaaaagtagttataaaaataaagaaatcagttcttggatatccagtgtgatacatattatagaagtttaaacagttactgttagaaactatattgggattctaaaaactaaagtgctctcctataactatgatatggattaacctcagaactaagtttaaattacagacctttactgggacaatgatcagaacatctgtatctgttacgataggttctataatgttcggaagatctttattgaaaattgaatggacttagagattctcctatatctagtataaatggacattcagaatcgaagaatagacgcagaaaataggctcaggtgagtttctaacatagagaattatgagatagtttacactattatgttaaaaaacacagatgatataaatcacatctattctttagagatgtattaaacaaatttaatccgagtttaggcttctaaacattacgaactaccattccagaatcgtatggactgctacgtgagggaaggaaattaagagattgataggttagtacttgaaaccgaaagagttgatcttaggtaaaaagatagaattatgataggagaaatctattaccttcttccaaacggttatgacataatgataggagatattatagtctaaatcctagaaaactatagtctatgtaaagacatctccgacccaatctacaaaagttgtgataattcgactgtagagaggtcatagaatttatgtttgaattaacaaaacaggtcagacatggattagaaccacgttaaaacgaatgattctattgcaggaataggggttgttccatacgttaggactagtgtgatattacaagtacattctggtggactagtgtaaacacaagaggtggaaggtacagtgttacttatttgggataacttaataagacattaagtaaggtctaatcacaaagtgtaagtagtaagggataggttcaactaggtcctaagggtaacttattttggaattgagaaactgtatttgagcactgtaataagttaaagaagggatggtcacggtgaagacttcctatttataaattaagagaaacaggcaactctagtgttcttaattgtggacttaatattatcaagtacgtcccaggattacatcgtagtattgttctgtcgtaacgtggacgtggaagaggctctatattagatagaaataggaataactgatctcctaagtcatttatccattcaaggttacgaaagtttgttgaccacgataattagggcttttcagagttaaccacactatttatagcgggtagttttccatctcaattaggatgtggactagaaggagaaagaagggaaaaatagagaagtattggagattcagcgtcttgaacactaaaactatgggaattgggggtcttccctcaagaaagcaagaagaaatttccatataacacaacttgtcaatataatagatcatcgtatgatagccgtagtcctagggtcaggagattctgaagaccatagggatatggagaatgagagttgtggaattaacttctaaaggactacattcaattgcaacttcccttatagaattattgattcggacagttaactgatatagaattatatcctagcagattttttcattcaagatctatgagaaagttactaagctcagcaagctctctacagtatctaaggttaaaaccagggagaaatcgatagagacggtttgatgtgttcatacgatctacagatcgatttttactttcaaaagagtaaagacgatataaacccagcaatagtgtttatttataaagttcgccatacgggagaacggttagtgcgtacttctccagttgtctcataagtgtatgactttaccgattgtcatagaacagaactaacctgttcaatccaggtatttgtccgagatgttgaggacgtagggtcttagcaaacttataaaatcctacactattacgtaacctgtcatgaaaatcctgacgtacactacacagaaattaatacgcatagtgtataggggtataaagtagggagggagactgcaaattgggcatttcacttatacgtttaactaactaatcttatgggggctttcaccttgttttattcccatatagttattggtatttaagccactaatttgataaatatagtagtagtaaatagtcacgatagtaaagaaattctaacagagatagattaactctgtaacacagacgttatcagtatcgacatgtatatgatcaacgagattaataaagtagtagtctggttattaagtatagttaacacacatgatgtagggattggaatgatttattgagatctagatacaggaactctcctagtcctagtatctaattgaacaataaaaggactcactagagattcaggttacatctagatcctccttataagttaagaactcctctaagatactatgtcgtaaataataagtttacattcacattatagtatccaaatctattactaggaacaaatatggagtataacttaagtttattcgagttaggccaattgtattaaacaactatttgatttaatcatagaaatctgagaggacggagaaaattacggtataatagtaatctataccattatttagctgagcgagcttgatttcatcgtgaacttttaaagtagacacatcgtcatagagagttagacaccctgtttaaacatcttcagctatgtccccactcattgaagtttaagagatttgaattcaatcgggaccaaactcgatattcacgacgaaggtaggtactataaagtagtcaaggtattcgggttcacatttggtaacgctaagaataacgacgaaaacggccgaacgaattcaagaattgtattggatcaactcggagactagaaagtagtcatcgtcttggttttattccctatgcataccgtcctgaaggtaacgattcacatagattcagctacaacagtccttctatcttggttcatatgtatcccaataagggaagactagatgttttaaatgtataaagactcggaagttatttactaggagaattattaagattgcccagacctcaaagatttggcacctaaccagaaggtattcaatctactttggtgtaaaaaacggagttaggataacggttaagatgacttgtgtatatgagtataagatataataggaattggccaaaagactcacaggaacaattccagagtataacattatctagtattaaatattcagaagaattctatgatatactattaggaggagaggactgtgatcgagaatgatctctaaagcaacatagttcgtagggccgatatcgaagagaatgtggacattcactcaatagattctagagcactcgatgagacccgttataccgaaaggataggtagtttttactacgattcaactctagaagaagtgatgaactaagacttttctctggactatcctctcctaatccactaaggacgttatgactagatcgacaatgtaaaaactaatatcaacaatatgaaacactcacaccctctaacgtacttattcccagactacgggttagattcttcgaactcagaggaccaagaactaagtagtgtgctatattatggacaaatagattgtctggacgaaactacttagagaaattcagacgatctcgacgttgacgacctagtggatataaagactgttttgtagatcgacatctgtatatcaacttgggaggatacaatcgaccctagttataacgtgtagaattggttaaaaagggaaattttataactagaaactatcaactgcattatcatcctaattaccagtaaggattactatatgtatggacaaccgttcaaaatttttctcgttatgttatcggattctatccacttataggtaagagttatcgaaaccgatatctacaacgttacaaactcgtacgactggacaaaagtagatgttctcagagactggttttatgtgtagaacaatttcgaaactccgtgacaccctcctaaaagggtagtattatataggcaaacgacatatgtttgtaaaacgatgaatattaccagagtaaattaaaattaagtacaggttatagtttgtattgaagagagttccgtggctttatagaacactaaagaagtatctgcactcagaaaaaaacgaatatccaaacacactgtccttgagacctacagtgacgatatcgaactaacagaggaacatggtaacgactgtgagactgtggttgaaattgtcgacgttctacctaacgtgattatctctaatcacaggtattaaagaccgttattgggagataagggggagaacctaatacctatttctaaggccatagtagtactagaacctcaagaaacacgtaagatagtccatgacctgtcattcctagaggatgtatttaacatgaaaaaagatcttatcccacgtaggtcaatttctttcaaaatttgggcttatagagtaatgtagaaactggcttgttgcgacatgaaagtttagaggtcaactctgtcataaaaaagtttagccaccaattttttgtcgagtctcagagtatggggagccaactactcagacgtcgaaacttaagtaaaaatctaaagaaagaaagtagggttatgggtaaacttaacgtgaataagtataaaaactagttaaagagtacattataccctaataacatttgactcaacctagaaccttgtggactctgtctattacatcagtcagagaaatcatccagttaaagagggaaatggtagggtaaaagtgacttattgagtggatgaggaaaacgatcatcacaaagacgattatggacctgacgagagtagaatatacagtagaaccgtttctcagctgggagaacaaattaaagaaaaagaaactctgacattctatacaacttcgatagcagtaaattggtaagaggactgagatgtattaattattataggcgtcccaattttaataatagtaaatatttttggagctaattggatgctcagagaagactaagtccctgaaacattatatttaatgacagccctatatgacttagggtacgacgaaaggatccactatctcggaataggaagtatatttaacagtttagaagtagattaactccgagttatttaaaatttttaaatttaggatattttgatgatattaacagatgccgtgtaagtatgctttaacgtctcaacctaggaacacgtaagaactcaagttctgtatgtactaaccccttaagcgtaccgccggtaactggtggcacagaaagagatataggcaaataatattaacgtgtcttctaacgtactgtgagtatatcacagaaatcatactaaaaaaacctccttatgtatacacaggaatgaaatagccgtcgtcaataacgaagattcgatcctacaggtttgcaagattttttcctcttttaaagacgaaaaagtagttaactacagagagacttttagaaatagttactaaggtaatacgacagacgaagacttagacacatatataatagaaaataattttaacatacatcgagtcagttttgtacagagtactttcgaggaaaatttaatcaatgtcccagtaggttgacataatttcgttcactatttccaagatcatcactatattataatatatgtaggagtggttctaaccctttatttctcagctattcgagggtattaagaagggggaatgtacaataaaaactactggaaaatagctcagcaggtcgacttctgtataaggtagaaggaagctgttgtagtgttatgtaattatgattaagtccacattcctgtattggccaattaaaattaaataaataataatagtgatttagagatatatattagttttatatactaaatgtcagttcttttaacactcttaaactctgtagatttaccaaggggctgaataagaaaacgtaggtagagtatagtataaactacgatttggtacaattcttacccgctatggtacacaaaaataatataaatagtcaatatagaccctactatagatgtaggaaattactcagtttagggagaaagggaagttgtgtcggattaacagattcttataaattggtgttaagaactttaggtctactatcaagagaataattagatagtctctgacttttttacctagaataaaacagtgtataaagaccctataggtcagactcattgagattactcttttagcctattccaagttttcacattgctagttataggaattaaagaacaaattcctaaaacggatcgctagactaaaactagacggccccgctatttggaggtaaagactaaaacataaaccactataaataatatgacaatagaaaatttagtatcccaactaactatagatcgttttgcacttcaactcgttagaacgaaaaatgttagcctctcaagttcactgtaagacctatattctacagactccttaagactcaactgaggacaaataggtataaactgtttatccgtccgtaattgggattcaaaaagaataagataaacagtatatttacagataagtacgaaacaaaaatttaaaaatatgtagtatagaccaaaagtgtattaactatatcacaacccagaactagacgagttctacactaaaatgtataaaatccctatgaacagaacttgttgtatccaacattccataattccgaccgaaccaactaaagttgttacaccttcgtcatcgggaagggctttactcactatgtactacatcatcacatacgacggagatcaacatgtaagaactcagaatcgtacaactattaaagactccataaactcatgtaataccaccctaattgtgcactacacaaacgcatgtcacaccaacatcgttgtaactgacgtagtcctctatcacctatacgtagtcatatatgtggactatacgtaagagagcctgtagacaacatggtcaacgtcagaaccaaaggaccagctctgtcctgaagtactccgcgggtaaattaccagtaccccaacaactatagattactaggataaacgtccaacctcacggttggacttctagatcatatatacatctagaaaaatggatcaaattcatcggatggaagccgtggattcattaaaactcaataaccttaacagagctgttgttagaaaccggatagtctatttattaataactatgcttaattctgtaactgttgaacagaaaagaaatcggttcaatagaattctcgtagtaacgtttgtgagactaattgtaaccatgtagacaaatagtgtgtgaatcacagcggaacctcgcctcaacaattcggtggcatgggatccttttactcacataaaagttaaaaatagggtgaaggatgtgagcctatgtaacgtctttttcctaccagttttcaatatagaagtaaaaacatagctactctaaaccagaaagggaactctagattatacagtttatgatttagaagatatggaagtgagctcatcagacatcagagtaagtgtcacccgttcctcgtattgactattggggaacaaggacgtcgataatgtcttgtactaaaggacaacagcaactacagtatccatactctttaatggcccaaatttagtcctatgtatagacttaaattcctatacattggattaacattttggactatactgaagggatatagacgtgtaggaactcactaattcaaactacttatgcgtatataacgtagtggttaactattacttccatcagattgtgtaggactttaacaccacctaggtctttcatctgaaccaggtccattattactctataaccccaacaacgagtcatccccaaggggatgccctgtagaggtctttagcacacccaggtctcctctatacggcagacgtaccacttatcgttaccacaaaagcgtatagactcggttaaggacagacaacgtactgaagagataattaacacactctagagacgaaacgaacaaattgttataaccctagggcgaaacatgacttgaaacatacaaataccaggaactataatgaagaacagactaattaagacactaacagagaaactaataaaaccgtgaaagcaagtacctcctatgacagtactacaaacagaagtatgttcacagaacaaaatattaatccagttacgtatcctagtactattacttcctgttatgacatacgtaacaacaacgttagtcatcggtctacacacaggaaggacgaccacataattacacacacaaatggcaccttaacagtaaagcccaacatctggttatacttgataaacaaaagggaaaaagtcggtagcaaacatattagcctacgacaacggaatatccaactgacctaacagaaattgggattcaaaaagaatgaacaacatgtggactaaccacttcaacacctatctaaactaacaacatacatacgatgttacatgaactagactaaaaaattaacctcaacaatgggtacatccctaaagagtaagaactgaggtctcatattcgtaattgtccacttaatactcaacttaattagtaatgattatcagaagattgggacatatctatcatattatatggtgtatactaataatattcatacgtgtgttaataatagtactaatagactcatgagagacacaacacggtaggaggttgacgactatactaacgggaacggaagtactcgagacagaacctaaggagatcctttaaacaccgtctacgttctaaatttctttacagatgtccagattacctctgacgacctgggtgaaactactaaactaagggggttcatcacagagcaggggaaaataaccgtatatcaagataaggtaaatatggataatttggcgttaaccatatccagttcttacattgaggagaactcgctagaactaagtgaccataagacaaacagggcgtacatgtacacctacgttacgttaatcggtgatgtggtggtaactacttacgcttgaaaccatgatctaacaaataatgaaatcagtgtcccgtgaaactatgtaggggctcatatgtgaagacaaccaatagtccataattaacacatcctagagatcctgtatatatacggttaaaactaagatatgtcaaacactgtagacgtggaggattcctactacggaataactaatacattaaaccctaacgctgtacggtaagaagaggaagttataatattctatatctacttcgagacatatagttgtgcggaccataaagactattttaaccataaaattgattctcccattggtacataaaaaggtttagttgtagatagtgtcaagggaattggacaagtcacatttactacagtatatatagactaaataggaaaaatcaacatgaatcttaaagacaatataatcgtctcatctctctactattacggacgttccattctgaaaaaagaggatatcagggttctaacctcgacttacgacaacgattaagtctcatcacaacccactcgaactagggatcaaagttacggcatcagagtgtggaatcaagaggataacgtccagaatagagcagtaaatgttttagaacattacagaaattacgttaatagacaagaggatatggagaaacttactcaagatgtcttaacacacagaactgataccttagaaaataatcacgatacaggaaggaccgaagagcacgaagtcgattacgttacgggcgtcaataaactcgtcgacaccgatgaggatcacgataccatggttactgtcgtggtttcttagatcaaacacctaatagtaaacagtgtcaccacagtaatcaataacaatagtccctaaggacttctaggttccgtaggaagtctccttaatcttcagataaattatttaagaacataacttattaaactcaacaaggtgtaggtagaacatccagttaacttccatggttcgaattgtgatacatagatctaagtattctaggacgataaaattcattaaacgggagtaactaatattggggatgtaaactttcaaaaagatggccttaaactgtattactacttctattatcgttcgaactcatatgattcttctcctagagactaaaaacgtaaaaacagagatagaaccacaaataatacttaaatttaagacaaaaacaaatttaaaaacaagaacggaggcaaaaacagagaccggaaaaaagaaaaaaccaaaaacacatatgaaaacaaaaagaagggaaaaaacaagaacaaaaacaaaaacgaaaacaagaacaaacactagaacaggaaaagaaagaaacacaacaacaaaaagagagacgaatccgactcgaaaaactctgagaacagaaggaactacaaccatctcctgaaacagggatgcaaaaagaataaacttgactaacatagcttaactagacataaactaattaccatgctaaatctcgattacccacagtaataactcctcacatctgaatataggaggactaaaagggctacgaaaaacgttgttgtaaacccattatcgctttaagaaagccttaacgtccaaccttgtgtcgtagatgtgaacactaaagatgacttcgacgggtctactgatctaagtatacacctaaatgtaggtaatttcctatcgtttgacgaaaaaaggtacgttcaaccgagtaatctcaaaacctattacacggacaacgtacatgtacctttgaataaggtggatgttcgggctctctcttataatcgaagtagaagagttaaaataaaaacgtcataagttgacttatatagaagggttgaaaagaagaatactaaggatccacatggtacttcaactcgaagaaaagaggtagtagcagatcttgcccctcctaaggaaaaacaaggcaactatgaggacgggattcacattggtcgtctaagtgactataactcaagccattacgttctagacgttctcaaaaaccctggaatcgttaacattaacatggatcactacaaggtaactgtttatgttgggacttagactacagaaacagctgaccctctgtaactccacgatctcgatgaaataaccgtaatatatagtagggaacagagtcagccgacctggtaccatcatctcctcgtacctagcttagatggtacatatgatagtaaagaggtctagattgacaagaagcatgtcaataaaactccagttacgtccgaaaattattaagaactagacacggcattatgaaccgatacggttaaccattcctaggcctaggtgtctaacttattgaccaactaagacactctagtctatacgacggtctatctccaggaacgaatccacaaagtttcttagggttatcatcgtgtagttctatggactgaggtacaaaacctcctagaggatggaactgataagactacactccctaacgaaaaaacagtcctggacaaaactctccctctccaagatgacagggtaaaagaagcacactcttagaccccttggacatacaaagtcggtaaaggaaagaggcccgttttaaagaaactgggattcaaaaagaattaacaaaactccctcgaaaaactcaaccgagacctataactaaaactaacgaccaactttaacatctaagtacaaatcaatcaatcacttagatacagaaggagcttatcgaggtaatgtaaaaattggagaaccaaacaggctgtgaatatttccctaaactacattcggaagaagagatgagacgaatctcctgacaaaagctattgatcaaactcactcacgtctcacacaaaaagtcacctgccttagtccgcactacgtaacctacgaactccgagtaacaacaaaagaagatcatggcctaaaaactacagtagagagtcaagtaggagagaatagttcagtccgaacttgagaactggaggatctcgaaggtatctccccagtttggaacagaaaaactggaataacagaaagggaaaacacttctgactaccactggaacatcatagtgaccctaaagggcaaaagggcggagatagacattaatatacctcacacctatctaaccggtagtactcactcaagacaagaaaaaccataaggctcttggcaaacacttctagggacctacttaaataaaggagaacttaaacaaattcattacaagataaaagaagatgaaataagagttcactaaaaagttgactatattcgtatggtgtctctaatttacagtaaagaaacattaaacgtctaaaattgtacgcgaatcgtttatgtactgaacgtgaggatctattaagtttaagtacaacactaaagtaatgtggttctgaatgattattgaaccggtagagtagctgactacacaacgaccaaaggagaagtaaacgccaacgcagcacaagccctcaaccacctgaaaaacacaacaacaaacgtaacaaacaacacatagaacaaaaagacaccaacaccaacaagaaacctcaccgacgactgagaagcaagaaactgccaccttgaaaacccgaccaaaaccacaacaccagaagctccaacctcgataaaagtgaacaactaaacacctcaagacttgagacacaagagagactagaacaaaccaatgcgaactaccagcagccggcagaaaacaaggaaacatcttgccgtagaagttcgagacctgaacccaaccattactgtagtcaataagaacatgatacacacgacgatcaaaggtacgaagaaggtaataataaacaagatgggagaggtagtagtctattcccacatgaagaagcatggagaagaccgagttgaaccagaagaagggagaataatctcaagagtcggtagaaagacagaagtaggcattcatgactagagactacagaagagagtaaacctcttagaagcagctaagaccacaaagaaagtaacgggtataagagtccatctcccaaacctgatcaaggagcggatctccgggacacatatggaacaactagttcctaagatcgtacacagagacaaaaactgagtgaacaagaggatctctaaggtgaggggagtgaactagatactagaacccagctgcaactctaacgagacagaaacaacaatagcagccgaaacgtactaccgaacaaaagaaaacccaacagctactatggtacctcggtcgaagacagaagaggttaaagacacccgaacatcctattctgctgcagttcctcaggctactgtagactctcactaagaacaaaagcaccacggagaggaagccgaagtcctagggagagttttttcttccgtaggactaggtaagcccttcctctcaaaacctacgactccataggtgacacttccaaccaaagcacatacgaccaccacagaccaactcgactagacacttaacagtaagtgggattcaaaaagaatgaaaactagaccgaagataacaggttcataggacaatttaaggaggatagggacgtcggcgacatcgtagtagtagtaagagtatcagtcgtagtgacgaccacagagcaaataacaaacggaccgaatcagagacaaaagacagccggtcggaagcacgttagaacaaaagatatagtcactacagaagtagtaaacagagaagaagttcaagaagacgtggtgcttagcactaacacaccaacgactagctcactaagtaggtagaaagtaacgacagtaaacaggtcaagaacaacagggctagtcgtcacaggagctgaggtttacaatacagacatactagttaacggtgaagatatcgaggtggtggacatccgaacaccatgcgtggtaggggaggactttccaaacagtctaccacagaatcagagagaaaacgacatagacagtgtggatcaagaaggaggtctcgtgacgactaaaaccttagtcgtagaaatcgatgtcgaacaggttcatccttgtaaaggtacaggtttatacaagaaggtcaatgtatgacaacgtatcgaaacaaaacatggtgtcgttgcgggtaacgcatcgaggtgtcacgacctatcaaaggcccccgtttcagaggtacttgacccagagactcatatgtatattttccccgagcccgagggaaactatctatctatagttgttccgaagagttaaacaaatatagaccagagtctaaactatcacagtcccgccggtagaagcaaagatgtggtatgaactaccacaagtactttctacgattaggacgtagagaatacattaaaggatgttagacttacaagaagagattacatcaatttagactggacctacacaagtatcagtggtctcaaagatggtattctctatgattcgaaacaacactggagtagtgacgtggttatggaagttgacacagaggacacttctggtcccgtgaaaattgccaaggtaggacagacttacgaagattagataacttcttaggaaaagaatctggtcgtgatgaacaataccgaaattggttgtgataggtttgaacttgataatatcgaggctctgtacgtcctataggtatacagacttcgttacggagatctaggcgtaagagagaaacgggaaccttgttacccaagaacaactagtatccaggtttgttggtgagacaacaagaaagtatgaggtacagagaacaaaactgttacttgggtagacaaaaacaagaaaatcctagagagagatataacatatagtgtatgaactgtagtcgtaactgtggcaaacatcactctatattgaggcctgatgacatccggtaacgttcctcactatgatttttaggaggagaaactctcacaacaaatagacacaggttactcacccgattctttcaccaacgataattattaaatagacgtagtagacagtgtgagcccggattacacttctgtctttgccatgaagaaactggtccatattaacgaggaggcggtctgaataactacgagagtgaagaggaccttgatttacacagttttcatgaatcatcgggccggtaatatttttaaatatgattctagtttacagacactgaacagggaaatttcataatgaaattgggattttatgataaattataataatatataaggtttgttcaaaaaggagaacaaacca

SEQ ID NO: 3 - protein, 18 aa, Artificial Sequence
  Peptide derived from HPIV1
QMREDIRDQYLRMKTERW

SEQ ID NO: 4 - protein, 15 aa, Artificial Sequence
  Peptide derived from HPIV1
RDPEAEGEAPRKQES

SEQ ID NO: 5 - DNA, 15474 bp, Human parainfluenza virus
  misc_feature (1)..(15474): Antigenome of the HPIV1 P(C-) virus
tggtctgttctcaaattctttatagctatataataattatgagaacagacaacattcaaaaagaatgatacaaaaatactcagtcaatttccctaaagaatagaacaattgagtagagaaatttgtttaatctattatttctactcttctatattctagtaacatacctcaggctacatagctataatcactatagtcgtctacacgagacagaacggggtttattaaactgacgagggttttaaaagtagttataaaaataaagaaatcagttcttggatatccagtgtgatacatattatagaagtttaaacagttactgttagaaactatattgggattctaaaaactaaagtgctctcctataactatgatatggattaacctcagaactaagtttaaattacagacctttactgggacaatgatcagaacatctgtatctgttacgataggttctataatgttcggaagatctttattgaaaattgaatggacttagagattctcctatatctagtataaatggacattcagaatcgaagaatagacgcagaaaataggctcaggtgagtttctaacatagagaattatgagatagtttacactattatgttaaaaaacacagatgatataaatcacatctattctttagagatgtattaaacaaatttaatccgagtttaggcttctaaacattacgaactaccattccagaatcgtatggactgctacgtgagggaaggaaattaagagattgataggttagtacttgaaaccgaaagagttgatcttaggtaaaaagatagaattatgataggagaaatctattaccttcttccaaacggttatgacataatgataggagatattatagtctaaatcctagaaaactatagtctatgtaaagacatctccgacccaatctacaaaagttgtgataattcgactgtagagaggtcatagaatttatgtttgaattaacaaaacaggtcagacatggattagaaccacgttaaaacgaatgattctattgcaggaataggggttgttccatacgttaggactagtgtgatattacaagtacattctggtggactagtgtaaacacaagaggtggaaggtacagtgttacttatttgggataacttaataagacattaagtaaggtctaatcacaaagtgtaagtagtaagggataggttcaactaggtcctaagggtaacttattttggaattgagaaactgtatttgagcactgtaataagttaaagaagggatggtcacggtgaagacttcctatttataaattaagagaaacaggcaactctagtgttcttaattgtggacttaatattatcaagtacgtcccaggattacatcgtagtattgttctgtcgtaacgtggacgtggaagaggctctatattagatagaaataggaataactgatctcctaagtcatttatccattcaaggttacgaaagtttgttgaccacgataattagggcttttcagagttaaccacactatttatagcgggtagttttccatctcaattaggatgtggactagaaggagaaagaagggaaaaatagagaagtattggagattcagcgtcttgaacactaaaactatgggaattgggggtcttccctcaagaaagcaagaagaaatttccatataacacaacttggagtcgtcaatataatagatcatcgtatgatagccgtagtcctagggtcaggagattctgaagaccatagggatatggagaatgagagttgtggaattaacttctaaaggactacattcaattgcaacttcccttatagaattattgattcggacagttaactgatatagaattatatcctagcagattttttcattcaagatctatgagaaagttactaagctcagcaagctctctacagtatctaaggttaaaaccagggagaaatcgatagagacggtttgatgtgttcatacgatctacagatcgatttttactttcaaaagagtaaagacgatataaacccagcaatagtgtttatttataaagttcgccatacgggagaacggttagtgcgtacttctccagttgtctcataagtgtatgactttaccgattgtcatagaacagaactaacctgttcaatccaggtatttgtccgagatgttgaggacgtagggtcttagcaaacttataaaatcctacactattacgtaacctgtcatgaaaatcctgacgtacactacacagaaattaatacgcatagtgtataggggtataaagtagggagggagactgcaaattgggcatttcacttatacgtttaactaactaatcttatgggggctttcaccttgttttattcccatatagttattggtatttaagccactaatttgataaatatagtagtagtaaatagtcacgatagtaaagaaattctaacagagatagattaactctgtaacacagacgttatcagtatcgacatgtatatgatcaacgagattaataaagtagtagtctggttattaagtatagttaacacacatgatgtagggattggaatgatttattgagatctagatacaggaactctcctagtcctagtatctaattgaacaataaaaggactcactagagattcaggttacatctagatcctccttataagttaagaactcctctaagatactatgtcgtaaataataagtttacattcacattatagtatccaaatctattactaggaacaaatatggagtataacttaagtttattcgagttaggccaattgtattaaacaactatttgatttaatcatagaaatctgagaggacggagaaaattacggtataatagtaatctataccattatttagctgagcgagcttgatttcatcgtgaacttttaaagtagacacatcgtcatagagagttagacaccctgtttaaacatcttcagctatgtccccactcattgaagtttaagagatttgaattcaatcgggaccaaactcgatattcacgacgaaggtaggtactataaagtagtcaaggtattcgggttcacatttggtaacgctaagaataacgacgaaaacggccgaacgaattcaagaattgtattggatcaactcggagactagaaagtagtcatcgtcttggttttattccctatgcataccgtcctgaaggtaacgattcacatagattcagctacaacagtccttctatcttggttcatatgtatcccaataagggaagactagatgttttaaatgtataaagactcggaagttatttactaggagaattattaagattgcccagacctcaaagatttggcacctaaccagaaggtattcaatctactttggtgtaaaaaacggagttaggataacggttaagatgacttgtgtatatgagtataagatataataggaattggccaaaagactcacaggaacaattccagagtataacattatctagtattaaatattcagaagaattctatgatatactattaggaggagaggactgtgatcgagaatgatctctaaagcaacatagttcgtagggccgatatcgaagagaatgtggacattcactcaatagattctagagcactcgatgagacccgttataccgaaaggataggtagtttttactacgattcaactctagaagaagtgatgaactaagacttttctctggactatcctctcctaatccactaaggacgttatgactagatcgacaatgtaaaaactaatatcaacaatatgaaacactcacaccctctaacgtacttattcccagactacgggttagattcttcgaactcagaggaccaagaactaagtagtgtgctatattatggacaaatagattgtctggacgaaactacttagagaaattcagacgatctcgacgttgacgacctagtggatataaagactgttttgtagatcgacatctgtatatcaacttgggaggatacaatcgaccctagttataacgtgtagaattggttaaaaagggaaattttataactagaaactatcaactgcattatcatcctaattaccagtaaggattactatatgtatggacaaccgttcaaaatttttctcgttatgttatcggattctatccacttataggtaagagttatcgaaaccgatatctacaacgttacaaactcgtacgactggacaaaagtagatgttctcagagactggttttatgtgtagaacaatttcgaaactccgtgacaccctcctaaaagggtagtattatataggcaaacgacatatgtttgtaaaacgatgaatattaccagagtaaattaaaattaagtacaggttatagtttgtattgaagagagttccgtggctttatagaacactaaagaagtatctgcactcagaaaaaaacgaatatccaaacacactgtccttgagacctacagtgacgatatcgaactaacagaggaacatggtaacgactgtgagactgtggttgaaattgtcgacgttctacctaacgtgattatctctaatcacaggtattaaagaccgttattgggagataagggggagaacctaatacctatttctaaggccatagtagtactagaacctcaagaaacacgtaagatagtccatgacctgtcattcctagaggatgtatttaacatgaaaaaagatcttatcccacgtaggtcaatttctttcaaaatttgggcttatagagtaatgtagaaactggcttgttgcgacatgaaagtttagaggtcaactctgtcataaaaaagtttagccaccaattttttgtcgagtctcagagtatggggagccaactactcagacgtcgaaacttaagtaaaaatctaaagaaagaaagtagggttatgggtaaacttaacgtgaataagtataaaaactagttaaagagtacattataccctaataacatttgactcaacctagaaccttgtggactctgtctattacatcagtcagagaaatcatccagttaaagagggaaatggtagggtaaaagtgacttattgagtggatgaggaaaacgatcatcacaaagacgattatggacctgacgagagtagaatatacagtagaaccgtttctcagctgggagaacaaattaaagaaaaagaaactctgacattctatacaacttcgatagcagtaaattggtaagaggactgagatgtattaattattataggcgtcccaattttaataatagtaaatatttttggagctaattggatgctcagagaagactaagtccctgaaacattatatttaatgacagccctatatgacttagggtacgacgaaaggatccactatctcggaataggaagtatatttaacagtttagaagtagattaactccgagttatttaaaatttttaaatttaggatattttgatgatattaacagatgccgtgtaagtatgctttaacgtctcaacctaggaacacgtaagaactcaagttctgtatgtactaaccccttaagcgtaccgccggtaactggtggcacagaaagagatataggcaaataatattaacgtgtcttctaacgtactgtgagtatatcacagaaatcatactaaaaaaacctccttatgtatacacaggaatgaaatagccgtcgtcaataacgaagattcgatcctacaggtttgcaagattttttcctcttttaaagacgaaaaagtagttaactacagagagacttttagaaatagttactaaggtaatacgacagacgaagacttagacacatatataatagaaaataattttaacatacatcgagtcagttttgtacagagtactttcgaggaaaatttaatcaatgtcccagtaggttgacataatttcgttcactatttccaagatcatcactatattataatatatgtaggagtggttctaaccctttatttctcagctattcgagggtattaagaagggggaatgtacaataaaaactactggaaaatagctcagcaggtcgacttctgtataaggtagaaggaagctgttgtagtgttatgtaattatgattaagtccacattcctgtattggccaattaaaattaaataaataataatagtgatttagagatatatattagttttatatactaaatgtcagttcttttaacactcttaaactctgtagatttaccaaggggctgaataagaaaacgtaggtagagtatagtataaactacgatttggtacaattcttacccgctatggtacacaaaaataatataaatagtcaatatagaccctactatagatgtaggaaattactcagtttagggagaaagggaagttgtgtcggattaacagattcttataaattggtgttaagaactttaggtctactatcaagagaataattagatagtctctgacttttttacctagaataaaacagtgtataaagaccctataggtcagactcattgagattactcttttagcctattccaagttttcacattgctagttataggaattaaagaacaaattcctaaaacggatcgctagactaaaactagacggccccgctatttggaggtaaagactaaaacataaaccactataaataatatgacaatagaaaatttagtatcccaactaactatagatcgttttgcacttcaactcgttagaacgaaaaatgttagcctctcaagttcactgtaagacctatattctacagactccttaagactcaactgaggacaaataggtataaactgtttatccgtccgtaattgggattcaaaaagaataagataaacagtatatttacagataagtacgaaacaaaaatttaaaaatatgtagtatagaccaaaagtgtattaactatatcacaacccagaactagacgagttctacactaaaatgtataaaatccctatgaacagaacttgttgtatccaacattccataattccgaccgaaccaactaaagttgttacaccttcgtcatcgggaagggctttactcactatgtactacatcatcacatacgacggagatcaacatgtaagaactcagaatcgtacaactattaaagactccataaactcatgtaataccaccctaattgtgcactacacaaacgcatgtcacaccaacatcgttgtaactgacgtagtcctctatcacctatacgtagtcatatatgtggactatacgtaagagagcctgtagacaacatggtcaacgtcagaaccaaaggaccagctctgtcctgaagtactccgcgggtaaattaccagtaccccaacaactatagattactaggataaacgtccaacctcacggttggacttctagatcatatatacatctagaaaaatggatcaaattcatcggatggaagccgtggattcattaaaactcaataaccttaacagagctgttgttagaaaccggatagtctatttattaataactatgcttaattctgtaactgttgaacagaaaagaaatcggttcaatagaattctcgtagtaacgtttgtgagactaattgtaaccatgtagacaaatagtgtgtgaatcacagcggaacctcgcctcaacaattcggtggcatgggatccttttactcacataaaagttaaaaatagggtgaaggatgtgagcctatgtaacgtctttttcctaccagttttcaatatagaagtaaaaacatagctactctaaaccagaaagggaactctagattatacagtttatgatttagaagatatggaagtgagctcatcagacatcagagtaagtgtcacccgttcctcgtattgactattggggaacaaggacgtcgataatgtcttgtactaaaggacaacagcaactacagtatccatactctttaatggcccaaatttagtcctatgtatagacttaaattcctatacattggattaacattttggactatactgaagggatatagacgtgtaggaactcactaattcaaactacttatgcgtatataacgtagtggttaactattacttccatcagattgtgtaggactttaacaccacctaggtctttcatctgaaccaggtccattattactctataaccccaacaacgagtcatccccaaggggatgccctgtagaggtctttagcacacccaggtctcctctatacggcagacgtaccacttatcgttaccacaaaagcgtatagactcggttaaggacagacaacgtactgaagagataattaacacactctagagacgaaacgaacaaattgttataaccctagggcgaaacatgacttgaaacatacaaataccaggaactataatgaagaacagactaattaagacactaacagagaaactaataaaaccgtgaaagcaagtacctcctatgacagtactacaaacagaagtatgttcacagaacaaaatattaatccagttacgtatcctagtactattacttcctgttatgacatacgtaacaacaacgttagtcatcggtctacacacaggaaggacgaccacataattacacacacaaatggcaccttaacagtaaagcccaacatctggttatacttgataaacaaaagggaaaaagtcggtagcaaacatattagcctacgacaacggaatatccaactgacctaacagaaattgggattcaaaaagaatgaacaacatgtggactaaccacttcaacacctatctaaactaacaacatacatacgatgttacatgaactagactaaaaaattaacctcaacaatgggtacatccctaaagagtaagaactgaggtctcatattcgtaattgtccacttaatactcaacttaattagtaatgattatcagaagattgggacatatctatcatattatatggtgtatactaataatattcatacgtgtgttaataatagtactaatagactcatgagagacacaacacggtaggaggttgacgactatactaacgggaacggaagtactcgagacagaacctaaggagatcctttaaacaccgtctacgttctaaatttctttacagatgtccagattacctctgacgacctgggtgaaactactaaactaagggggttcatcacagagcaggggaaaataaccgtatatcaagataaggtaaatatggataatttggcgttaaccatatccagttcttacattgaggagaactcgctagaactaagtgaccataagacaaacagggcgtacatgtacacctacgttacgttaatcggtgatgtggtggtaactacttacgcttgaaaccatgatctaacaaataatgaaatcagtgtcccgtgaaactatgtaggggctcatatgtgaagacaaccaatagtccataattaacacatcctagagatcctgtatatatacggttaaaactaagatatgtcaaacactgtagacgtggaggattcctactacggaataactaatacattaaaccctaacgctgtacggtaagaagaggaagttataatattctatatctacttcgagacatatagttgtgcggaccataaagactattttaaccataaaattgattctcccattggtacataaaaaggtttagttgtagatagtgtcaagggaattggacaagtcacatttactacagtatatatagactaaataggaaaaatcaacatgaatcttaaagacaatataatcgtctcatctctctactattacggacgttccattctgaaaaaagaggatatcagggttctaacctcgacttacgacaacgattaagtctcatcacaacccactcgaactagggatcaaagttacggcatcagagtgtggaatcaagaggataacgtccagaatagagcagtaaatgttttagaacattacagaaattacgttaatagacaagaggatatggagaaacttactcaagatgtcttaacacacagaactgataccttagaaaataatcacgatacaggaaggaccgaagagcacgaagtcgattacgttacgggcgtcaataaactcgtcgacaccgatgaggatcacgataccatggttactgtcgtggtttcttagatcaaacacctaatagtaaacagtgtcaccacagtaatcaataacaatagtccctaaggacttctaggttccgtaggaagtctccttaatcttcagataaattatttaagaacataacttattaaactcaacaaggtgtaggtagaacatccagttaacttccatggttcgaattgtgatacatagatctaagtattctaggacgataaaattcattaaacgggagtaactaatattggggatgtaaactttcaaaaagatggccttaaactgtattactacttctattatcgttcgaactcatatgattcttctcctagagactaaaaacgtaaaaacagagatagaaccacaaataatacttaaatttaagacaaaaacaaatttaaaaacaagaacggaggcaaaaacagagaccggaaaaaagaaaaaaccaaaaacacatatgaaaacaaaaagaagggaaaaaacaagaacaaaaacaaaaacgaaaacaagaacaaacactagaacaggaaaagaaagaaacacaacaacaaaaagagagacgaatccgactcgaaaaactctgagaacagaaggaactacaaccatctcctgaaacagggatgcaaaaagaataaacttgactaacatagcttaactagacataaactaattaccatgctaaatctcgattacccacagtaataactcctcacatctgaatataggaggactaaaagggctacgaaaaacgttgttgtaaacccattatcgctttaagaaagccttaacgtccaaccttgtgtcgtagatgtgaacactaaagatgacttcgacgggtctactgatctaagtatacacctaaatgtaggtaatttcctatcgtttgacgaaaaaaggtacgttcaaccgagtaatctcaaaacctattacacggacaacgtacatgtacctttgaataaggtggatgttcgggctctctcttataatcgaagtagaagagttaaaataaaaacgtcataagttgacttatatagaagggttgaaaagaagaatactaaggatccacatggtacttcaactcgaagaaaagaggtagtagcagatcttgcccctcctaaggaaaaacaaggcaactatgaggacgggattcacattggtcgtctaagtgactataactcaagccattacgttctagacgttctcaaaaaccctggaatcgttaacattaacatggatcactacaaggtaactgtttatgttgggacttagactacagaaacagctgaccctctgtaactccacgatctcgatgaaataaccgtaatatatagtagggaacagagtcagccgacctggtaccatcatctcctcgtacctagcttagatggtacatatgatagtaaagaggtctagattgacaagaagcatgtcaataaaactccagttacgtccgaaaattattaagaactagacacggcattatgaaccgatacggttaaccattcctaggcctaggtgtctaacttattgaccaactaagacactctagtctatacgacggtctatctccaggaacgaatccacaaagtttcttagggttatcatcgtgtagttctatggactgaggtacaaaacctcctagaggatggaactgataagactacactccctaacgaaaaaacagtcctggacaaaactctccctctccaagatgacagggtaaaagaagcacactcttagaccccttggacatacaaagtcggtaaaggaaagaggcccgttttaaagaaactgggattcaaaaagaattaacaaaacgccggcgcgatcgacatctaagtacaaatcaatcaatcacttagatacagaaggagcttatcgaggtaatgtaaaaattggagaaccaaacaggctgtgaatatttccctaaactacattcggaagaagagatgagacgaatctcctgacaaaagctattgatcaaactcactcacgtctcacacaaaaagtcacctgccttagtccgcactacgtaacctacgaactccgagtaacaacaaaagaagatcatggcctaaaaactacagtagagagtcaagtaggagagaatagttcagtccgaacttgagaactggaggatctcgaaggtatctccccagtttggaacagaaaaactggaataacagaaagggaaaacacttctgactaccactggaacatcatagtgaccctaaagggcaaaagggcggagatagacattaatatacctcacacctatctaaccggtagtactcactcaagacaagaaaaaccataaggctcttggcaaacacttctagggacctacttaaataaaggagaacttaaacaaattcattacaagataaaagaagatgaaataagagttcactaaaaagttgactatattcgtatggtgtctctaatttacagtaaagaaacattaaacgtctaaaattgtacgcgaatcgtttatgtactgaacgtgaggatctattaagtttaagtacaacactaaagtaatgtggttctgaatgattattgaaccggtagagtagctggctacacaacgaccaaaggagaagtaaacgccaacgcagcacaagccctcaaccacctgaaaaacacaacaacaaacgtaacaaacaacacatagaacaaaaagacaccaacaccaacaagaaacctcaccgacgactgagaagcaagaaactgccaccttgaaaacccgaccaaaaccacaacaccagaagctccaacctcgataaaagtgaacaactaaacacctcaagacttgagacacaagagagactagaacaaaccaatgcgaactaccagcagccggcagaaaacaaggaaacatcttgccgtagaagttcgagacctgaacccaaccattactgtagtcaataagaacatgatacacacgacgatcaaaggtacgaagaaggtaataataaacaagatgggagaggtagtagtctattcccacatgaagaagcatggagaagacctttagggagttgaaccagaagaagggagaataatctcaagagtcggtagaaagacagaagtaggcattcatgactagagactacagaagagagtaaacctcttagaagcagctaagaccacaaagaaagtaacgggtataagagtccatctcccaaacctgatcaaggagcggatctccgggacacatatggaacaactagttcctaagatcgtacacagagacaaaaactgagtgaacaagaggatctctaaggtgagaggagtgaactagatactagaacccagctgcaactctaacgagacagaaacaacaatagcagccgaaacgtactaccgaacaaaagaaaacccaacagctactatggtacctcggtcgaagacagaagaggataaagacacccgaacatcctattctgatgcagttcctcaggctagtgtagactctcactaagaacaaaagcaccacggagaggaagccgaagtcctagggagagttttttcttccgcaggactaggtacaacccgaaattgggattcaaaaagaatgaaaactagaccgaagataacaggttcataggacaatttaaggaggatagggacgtcggcgacatcgtagtagtagtaagagtatcagtcgtagtgacgaccacagagcaaataacaaacggaccgaatcagagacaaaagacagccggtcggaagcacgttagaacaaaagatatagtcactacagaagtagtaaacagagaagaagttcaagaagacgtggtgcttagcactaacacaccaacgactagctcactaagtaggtagaaagtaacgacagtaaacaggtcaagaacaacagggctagtcgtcacaggagctgaggtttacaatacagacatactagttaacggtgaagatatcgaggtggtggacatccgaacaccatgcgtggtaggggaggactttccaaacagtctaccacagaatcagagagaaaacgacatagacagtgtggatcaagaaggaggtctcgtgacgactaaaaccttagtcgtagaaatcgatgtcgaacaggttcatccttgtaaaggtacaggtttatacaagaaggtcaatgtatgacaacgtatcgaaacaaaacatggtgtcgttgcgggtaacgcatcgaggtgtcacgacctatcaaaggtccccgtttcagaggtacttgacccagagactcatatgtatattttccccgagcccgagggaaactatctatctatagttgttccgaagagttaaacaaatatagaccagagtctaaactatcacagtcccgccggtagaagcaaagatgtggtatgaactaccacaagtactttctacgattaggacgtagagaatacattaaaggatgttagacttacaagaagagattacatcaatttagactggacctacacaagtatcagtggtctcaaagatggtattctctatgattcgaaacaacactggagtagtgacgtggttatggaagttgacacagaggacacttctggtcccgtgaaaattgccaaggtaggacagacttacgaagattagataacttcttaggaaaagaatctggtcgtgatgaacaataccgaaattggttgtgataggtttgaacttgataatatcgaggctctgtacgtcctataggtatacagacttcgttacggagatctaggcgtaagagagaaacgggaaccttgttacccaagaacaactagtatccaggtttgttggtgagacaacaagaaagtatgaggtacagagaacaaaactgttacttgggtagacaaaaacaagaaaatcctagagagagatataacatatagtgtatgaactgtagtcgtaactgtggcaaacatcactctatattgaggcctgatgacatccggtaacgttcctcactatgatttttaggaggagaaactctcacaacaaatagacacaggttactcacccgattctttcaccaacgataattattaaatagacgtagtagacagtgtgagcccggattacacttctgtctttgccatgaagaaactggtccatattaacgaggaggcggtctgaataactacgagagtgaagaggaccttgatttacacagttttcatgaatcatcgggccggtaatgcgcataaatatgattctagtttacagacactgaacagggaaatttcataatgaaattgggattttatgataaattataataatatataaggtttgttcaaaaaggagaacaaacca